(19) United States
(12) Patent Application Publication
(10) Pub. No.: US 2012/0171768 A1
(43) Pub. Date: Jul. 5, 2012

(54) COLLAGEN-BASED MATRICES FOR VESSEL DENSITY AND SIZE REGULATION

(75) Inventor: Sherry L Voytik-Harbin, Zionsville, IN (US)

(73) Assignee: Purdue Research Foundation, West Lafayette (IN)

(21) Appl. No.: 13/265,466

(22) PCT Filed: Apr. 20, 2010
(86) PCT No.: PCT/US10/31791

§ 371 Date: Oct. 20, 2011

Related U.S. Application Data
(60) Provisional application No. 61/170,854, filed on Apr. 20, 2009.
(60) Provisional application No. 61/228,876, filed on Jul. 27, 2009.
(60) Provisional application No. 61/229,963, filed on Jul. 30, 2009.

(51) Int. Cl. C12N 5/02

(52) U.S. Cl. 435/395

(57) ABSTRACT

Collagen based-matrices and methods of their use are described. More particularly, collagen-based matrices and methods for regulating vessel density and size within collagen-based matrices are described.

### CROSS REFERENCE TO RELATED APPLICATIONS

This application claims priority under 35 U.S.C. §119(e) to U.S. Provisional Application No. 61/170,854, filed Apr. 20, 2009, Application No. 61/228,876, filed Jul. 27, 2009, and Application No. 61/229,963, filed Jul. 30, 2009, each of which is expressly incorporated by reference herein.

### FIELD OF THE INVENTION

This invention relates to collagen based-matrices and methods of their use. More particularly, the invention relates to collagen-based matrices and methods for vessel density and size regulation.

### BACKGROUND AND SUMMARY

Vascular network formation is a limiting obstacle for tissue engineering strategies targeting repair and regeneration of damaged or diseased tissue. Development of functional vascular networks is important for the treatment of various diseases, such as, diabetic ulcers, limb ischemia, cerebral ischemia, peripheral vascular disease, and cardiovascular disease. Therapeutic use of stem and progenitor cells for the treatment of diseases or dysfunctional tissues has been limited by the ability to control their survival, proliferation, and differentiation. Recently, three-dimensional (3D) extracellular matrices (ECMs) have been identified as an important component of stem cell technology to assist in guiding cell behavior. However, tissue engineering approaches with engineered collagen matrices to generate functional vascular networks, needed for the treatment of peripheral and cardiovascular disease, have not been previously developed.

The fibril microstructure of collagen-based ECMs, whether presented as parallel-aligned bundles as found in tendons and ligaments or woven networks as found in dermis, bone, and cornea, determines not only tissue-level mechanical properties but also instructive physicochemical features of the local cellular microenvironment. Cells sense and respond to the spatial distribution of fibrils largely through formation of cell-matrix adhesions through integrin-mediated binding of adhesion domains inherent to the collagen molecule. A mechanical force balance results as cells exert cytoskeletal-based contraction which is resisted by structural-mechanical properties of the surrounding collagen-fibril matrix. This interaction induces a number of physical and biochemical based signal transduction reactions, that ultimately guide fundamental cellular behaviors, including proliferation, migration, and differentiation. In addition, matrix remodeling and degradation, as determined by the fibril microstructure and type and extent of intermolecular cross-linking, are regulators of normal and disease state processes including tissue (e.g., vascular) morphogenesis, wound healing, and cancer cell metastasis.

Applicants have engineered collagen-based matrices with the potential to direct vessel formation. Mechanical properties including fiber diameter, fibril density, fibril length, and matrix stiffness can be modulated by controlling polymerization parameters including collagen concentration, temperature, pH, ionic strength, and polymerization time. Applicants describe engineered collagen-based matrices that modulate in vitro and in vivo vessel formation to improve the efficiency of cellular-based therapies to regenerate or repair blood vessels. Systemic variation of polymerization conditions such as pH, ionic strength, and molecular composition provides a means to control polymerization kinetics, fibril microstructure, and mechanical properties of 3D collagen matrices. These microstructural-mechanical properties, in turn, provide instructional information to stem cells, and have been used by Applicants as design parameters to influence cell behavior.

Vascular tissue engineering using ECM-based biomaterials to improve cell-mediated revascularization in damaged or diseased tissues has great clinical potential. As herein described, the physical properties of 3D collagen matrices influence endothelial colony forming cell (ECFC) blood vessel formation both in vitro and in vivo. Matrix physical properties affect implant revascularization and remodeling, including vessel density, proportion, and areas.

In one illustrative embodiment, a method of regulating vessel density within an engineered purified collagen-based matrix composition is described. The method comprises the steps of engineering the purified collagen-based matrix comprising collagen fibrils, and contacting the matrix with endothelial progenitor cells wherein said contacting results in vessel formation and an increase in vessel density within the matrix with decreasing collagen concentration.

In the above described embodiment, the fibril volume fraction of the matrix can be about 1% to about 10%, the storage modulus of the matrix can be about 1 Pa to about 15 Pa, the compressive modulus of the matrix can be about 15,000 Pa to about 50,000 Pa, the vessels can be formed from endothelial progenitor cells, the vessel density within the matrix can be about 40 vessels/mm² to about 80 vessels/mm², and the matrix can be prepared from collagen at collagen concentrations ranging from about 0.1 mg/ml to about to about 4.0 mg/ml.

In another embodiment, a method of regulating vessel size within an engineered purified collagen-based matrix composition is described. The method comprises the steps of engineering a purified collagen-based matrix comprising collagen fibrils, and contacting the matrix with endothelial progenitor cells wherein said contacting results in vessel formation and an increase in vessel size within the matrix with increasing collagen concentration.

In the above described embodiment, the fibril volume fraction of the matrix can be about 12% to about 20%, the storage modulus of the matrix can be about 25 Pa to about 50 Pa, the compressive modulus of the matrix can be about 90,000 Pa to about 150,000 Pa, the vessels can be formed from endothelial progenitor cells, the average vessel size within the matrix can be about 300 μm² to about 600 μm², and the matrix can be prepared from collagen at collagen concentrations ranging from about 0.1 mg/ml to about to about 4.0 mg/ml.

In another embodiment, a method of regulating the density of vessels within an engineered purified collagen-based matrix composition prior to implantation is described. The method comprises the steps of engineering the purified collagen-based matrix comprising collagen fibrils, and contacting the matrix with endothelial progenitor cells wherein said contacting results in vessel formation and an increase in vessel density within the matrix with decreasing collagen concentration.

In the above described embodiment, the fibril volume fraction of the matrix can be about 1% to about 10%, the storage modulus of the matrix can be about 1 Pa to about 15 Pa, the compressive modulus of the matrix can be about 15,000 Pa to about 50,000 Pa, the vessels can be formed from endothelial progenitor cells, the vessel density within the matrix can be about 40 vessels/mm² to about 80 vessels/mm², the matrix can be prepared from collagen at collagen concentrations ranging from about 0.1 mg/ml to about to about 4.0 mg/ml.

In another embodiment, a method of regulating the size of vessels within an engineered purified collagen-based matrix composition is described. The method comprises the steps of engineering the purified collagen-based matrix comprising collagen fibrils, and contacting the matrix with endothelial progenitor cells wherein said contacting results in vessel formation and an increase in the average vessel area within the matrix with increasing collagen concentration.

In the above described embodiment, the fibril volume fraction of the matrix can be about 12% to about 20%, the storage modulus of the matrix can be about 25 Pa to about 50 Pa, the compressive modulus of the matrix can be about 90,000 Pa to about 150,000 Pa, the vessels can be formed from endothelial progenitor cells, the average vessel area within the matrix can be about 300 μm² to about 600 μm², the matrix can be prepared from collagen at collagen concentrations ranging from about 0.1 mg/ml to about to about 4.0 mg/ml.

In another embodiment, an engineered purified collagen-based matrix composition is described. The composition comprises vessels wherein the vessel density within the matrix is about 10 vessels/mm²to about 80 vessels/mm².

In another embodiment, an engineered purified collagen-based matrix composition is described. The composition comprises vessels wherein the average vessel area within the matrix is about 10 μm² to about 600 μm².

The following various embodiments are provided.

1. A method of regulating vessel density within an engineered purified collagen-based matrix composition is described. The method comprises the steps of engineering a purified collagen-based matrix comprising collagen fibrils, and seeding the matrix with endothelial progenitor cells wherein said seeding results in vessel formation and an increase in of vessel density within the matrix with decreasing collagen concentration.

2. The method of clause 1 wherein the fibril volume fraction of the matrix is about 1% to about 10%.

3. The method of clause 1 to 2 wherein the storage modulus of the matrix is about 1 Pa to about 15 Pa.

4. The method of clause 1 to 3 wherein the compressive modulus of the matrix is about 15,000 Pa to about 50,000 Pa.

5. The method of clause 1 to 4 wherein the vessels are formed from the endothelial progenitor cells.

6. The method of clause 1 to 5 wherein the vessel density within the matrix is about 40 vessels/mm²to about 80 vessels/mm².

7. The method of clause 1 to 6 wherein the matrix is prepared from collagen at a collagen concentrations ranging from about 0.1 mg/ml to about to about 4.0 mg/ml.

8. The method of clause 1 to 7 wherein the matrix composition is used as a tissue graft.

9. The method of clause 1 to 8 wherein the vessels are formed prior to implantation of the tissue graft.

10. A method of regulating vessel area within an engineered purified collagen-based matrix composition is described. The method comprises the steps of engineering a purified collagen-based matrix comprising collagen fibrils, and seeding the matrix with endothelial progenitor cells wherein said seeding results in vessel formation and an increase of vessel area within the matrix with increasing collagen concentration.

11. The method of clause 10 wherein the fibril volume fraction of the matrix is about 12% to about 20%.

12. The method of clause 10 to 11 wherein the storage modulus of the matrix is about 25 Pa to about 50 Pa.

13. The method of clause 10 to 12 wherein the compressive modulus of the matrix is about 90,000 Pa to about 150,000 Pa.

14. The method of clause 10 to 13 wherein the vessels are formed from the endothelial progenitor cells.

15. The method of clause 10 to 14 wherein the average vessel area within the matrix is about 300 μm² to about 600 μm².

16. The method of clause 10 to 15 wherein the matrix is prepared from collagen at a collagen concentrations ranging from about 0.1 mg/ml to about to about 4.0 mg/ml.

17. The method of clause 10 to 16 wherein the matrix composition is used as a tissue graft.

18. The method of clause 10 to 17 wherein the vessels are formed prior to implantation of the tissue graft.

19. An engineered collagen-based matrix composition is described. The composition comprises vessels wherein the vessel density within the matrix is about 10 vessels/mm² to about 80 vessels/mm².

20. The matrix of clause 19 wherein the fibril volume fraction of the matrix is about 1% to about 10%.

21. The matrix of clause 19 to 20 wherein the storage modulus of the matrix is about 1 Pa to about 15 Pa. 22. The matrix of clause 19 to 21 wherein the compressive modulus of the matrix is about 15,000 Pa to about 50,000 Pa.

23. The matrix of clause 19 to 22 wherein the vessels are formed from endothelial progenitor cells.

24. The matrix of clause 19 to 23 wherein the vessel density within the matrix is about 40 vessels/mm²to about 80 vessels/mm².

25. The matrix of clause 19 to 24 wherein the matrix is prepared from collagen at a collagen concentrations ranging from about 0.1 mg/ml to about to about 4.0 mg/ml.

26. The matrix of clause 19 to 25 for use as a tissue graft.

27. An engineered collagen-based matrix composition is described. The composition comprises vessels wherein the average vessel area within the matrix is about 10 μm² to about 600 μm².

28. The matrix of clause 27 wherein the fibril volume fraction of the matrix is about 12% to about 20%.

29. The matrix of clause 27 to 28 wherein the storage modulus of the matrix is about 25 Pa to about 50 Pa.

30. The matrix of clause 27 to 29 wherein the compressive modulus of the matrix is about 90,000 Pa to about 150,000 Pa.

31. The matrix of clause 27 to 30 wherein the vessels are formed from endothelial progenitor cells. 32. The matrix of clause 27 to 31 wherein the average vessel area within the matrix is about 300 μm² to about 600 μm².

33. The matrix of clause 27 to 32 wherein the matrix is prepared from collagen at a collagen concentrations ranging from about 0.1 mg/ml to about to about 4.0 mg/ml.

34. The matrix of clause 27 to 33 for use as a tissue graft.

35. A method of preparing an engineered collagen-based matrix is described The method comprises the steps of providing purified collagen; providing endothelial progenitor cells; polymerizing the purified collagen into fibrils; and seeding the polymerized purified collagen with the endothelial progenitor cells, wherein said seeding results in vessel formation and an increase in the average vessel area within the matrix with increasing collagen concentrations.

36. The method of clause 35 wherein the fibril volume fraction of the matrix is about 12% to about 20%.

37. The method of clause 35 to 36 wherein the storage modulus of the matrix is about 25 Pa to about 50 Pa.

38. The method of clause 35 to 37 wherein the compressive modulus of the matrix is about 90,000 Pa to about 150,000 Pa.

39. The method of clause 35 to 38 wherein the vessels are formed from the endothelial progenitor cells.

40. The method of clause 35 to 39 wherein the average vessel area within the matrix is about 300 μm² to about 600 μm².

41. The method of clause 35 to 40 wherein the matrix is prepared from collagen at a collagen concentrations ranging from about 0.1 mg/ml to about to about 4.0 mg/ml 42. The method of clause 35 to 41 wherein the matrix is prepared from collagen with an average polymer molecular weight of about 350 kDa to about 2.8 MDa.

43. The method of clause 35 to 42 wherein the matrix is prepared from collagen with an average polymer molecular weight of about 350 kDa to about 700 kDa.

44. A method of preparing an engineered collagen-based matrix is described. The method comprises the steps of providing purified collagen; providing endothelial progenitor cells; polymerizing the purified collagen into fibrils; and seeding the polymerized purified collagen with the endothelial progenitor cells, wherein said seeding results in vessel formation and an increase in the average vessel density within the matrix with decreasing collagen concentration.

45. The method of clause 44 wherein the fibril volume fraction of the matrix is about 1% to about 10%.

46. The method of clause 44 to 45 wherein the storage modulus of the matrix is about 1 Pa to about 15 Pa.

47. The method of clause 44 to 46 wherein the compressive modulus of the matrix is about 15,000 Pa to about 50,000 Pa.

48. The method of clause 44 to 47 wherein the vessels are formed from the endothelial progenitor cells.

49. The method of clause 44 to 48 wherein the vessel density within the matrix is about 40 vessels/mm²to about 80 vessels/mm².

50. The method of clause 44 to 49 wherein the matrix is prepared from collagen at a collagen concentrations ranging from about 0.1 mg/ml to about to about 4.0 mg/ml.

51. The method of clause 44 to 50 wherein the matrix is prepared from collagen with an average polymer molecular weight of about 350 kDa to about 2.8 MDa.

52. The method of clause 44 to 51 wherein the matrix is prepared from collagen with an average polymer molecular weight of about 350 kDa to about 700 kDa.

53. A kit comprising an engineered purified collagen-based matrix is described, wherein the vessel density within the matrix is about 40 vessels/mm² to about 80 vessels/mm².

54. The kit of clause 53 wherein the matrix is lyophilized.

55. The kit of clause 53 to 54 further comprising a glucose and a calcium chloride solution.

56. The kit of clause 53 to 55 further comprising a buffer.

57. The kit of clause 53 to 56 wherein the average vessel area within the matrix is about 300 μm² to about 600 μm².

58. The kit of clause 53 to 57 wherein the matrix is prepared from collagen at collagen concentrations ranging from about 0.1 mg/ml to about to about 4.0 mg/ml.

59. The kit of clause 53 to 58 wherein the matrix is prepared from collagen with an average polymer molecular weight of about 350 kDa to about 2.8 MDa.

60. The kit of clause 53 to 59 wherein the matrix is prepared from collagen with an average polymer molecular weight of about 350 kDa to about 700 kDa.

61. A kit comprising an engineered purified collagen-based matrix is described, wherein the average vessel area within the matrix is about 300 μm² to about 600 μm².

62. The kit of clause 61 wherein the matrix is lyophilized.

63. The kit of clause 61 to 62 further comprising a glucose and a calcium chloride solution.

64. The kit of clause 61 to 63 further comprising a buffer.

65. The kit of clause 61 to 64 wherein the vessel density within the matrix is about 40 vessels/mm²to about 80 vessels/mm².

66. The kit of clause 61 to 65 wherein the matrix is prepared from collagen at collagen concentrations ranging from about 0.1 mg/ml to about to about 4.0 mg/ml.

67. The kit of clause 61 to 66 wherein the matrix is prepared from collagen with an average polymer molecular weight of about 350 kDa to about 2.8 MDa.

68. The kit of clause 61 to 67 wherein the matrix is prepared from collagen with an average polymer molecular weight of about 350 kDa to about 700 kDa.

69. A method of regulating vacuole density within an engineered purified collagen-based matrix is described. The method comprises the steps of engineering a purified collagen-based matrix comprising collagen fibrils, and seeding the matrix with endothelial progenitor cells, wherein said seeding results in vacuole formation, and wherein the vacuole density is about 30 vacuoles/mm² to about 80 vacuoles/mm².

70. The method of clause 69 wherein the fibril volume fraction of the matrix is about 1% to about 10%.

71. The method of clause 69 to 70 wherein the storage modulus of the matrix is about 1 Pa to about 15 Pa.

72. The method of clause 69 to 71 wherein the compressive modulus of the matrix is about 15,000 Pa to about 50,000 Pa.

73. The method of clause 69 to 72 wherein the matrix is prepared from collagen at a collagen concentrations ranging from about 0.1 mg/ml to about to about 4.0 mg/ml.

74. A method of regulating vacuole density within an engineered purified collagen-based matrix is described. The method comprises the steps of engineering a purified collagen-based matrix comprising collagen fibril, and seeding the matrix with endothelial progenitor cells, wherein said seeding results in vacuole formation, and wherein the total vacuole area is about 1800 μm² to about 5000 μm².

75. The method of clause 74 wherein the fibril volume fraction of the matrix is about 1% to about 10%.

76. The method of clause 74 to 75 wherein the storage modulus of the matrix is about 1 Pa to about 15 Pa.

77. The method of clause 74 to 76 wherein the compressive modulus of the matrix is about 15,000 Pa to about 50,000 Pa.

78. The method of clause 74 to 77 wherein the matrix is prepared from collagen at a collagen concentrations ranging from about 0.1 mg/ml to about to about 4.0 mg/ml.

80. An engineered purified collagen-based matrix comprising vacuoles, wherein the total vacuole area is about 1800 μm² to about 5000 μm² is described 81. An engineered purified collagen-based matrix comprising vacuoles, wherein the vacuole density is about 30 vacuoles/mm² to about 80 vacuoles/mm² is described.

### BRIEF DESCRIPTION OF THE DRAWINGS

Table 1 shows a summary of the specific collagen polymerization reaction conditions used to systematically vary fundamental fibril microstructure and viscoelastic properties of engineered 3D matrices.

Table 2 shows a summary of the relative expression of cell surface markers CD34, CD133, and PECAM in CBFs seeded within 3D extracellular matrices (ECMs) compared to seeding on plastic.

Table 3 shows a summary of collagen matrix physical properties.

Table 4 shows a summary of microstructural properties of 3D matrices prepared with collagen formulations with varied (average polymer molecular weight) AMW or monomer/oligomer content. All matrices were polymerized under similar conditions at 0.7 mg/ml collagen concentration.

FIG. 1 shows flow cytometry data for quantification of CD34 in CBF cells.

FIG. 2 shows flow cytometry data for quantification of CD34 in CBF cells.

FIG. 3 shows flow cytometry data for quantification of CD34 in CBF cells.

FIG. 4 shows flow cytometry data for quantification of CD34 in CBF cells.

FIG. 5 shows flow cytometry data for quantification of CD34 in CBF cells.

FIG. 6 shows flow cytometry data for quantification of CD34 in CBF cells.

FIG. 7 shows a histogram of the percentage of cells expressing PECAM, CD34, CD133, and CD45 following harvest by collagenase cocktail or trypsin and 6 days in culture in 0.5 mg/ml or 2.0 mg/ml pig skin collagen (PSC) (within each group represented on the abscissa; the first bar from left=0.5 mg/ml PSC, second bar=2.0 mg/ml PSC, third bar=collagenase control, and fourth bar=trypsin control).

FIG. 8 shows a histogram of the colony size formed (t=4 days) by an endothelial progenitor cell (EPC) population before being seeded within 3D ECMs (Ctrl) and after being seeded at cell densities of 1×10⁵ cells/ml within 3D ECMs polymerized at 0.5 mg/ml and 2.0 mg/ml PSC. Note the shift in the colony forming potential for the cells seeded under the different conditions. These data include single cell events. (within each group represented on the abscissa; the left bar=control, middle bar=0.5 mg/ml PSC, right bar=2 mg/ml PSC).

FIG. 9 shows a histogram of the colony size formed (t=4 days) by an EPC population before being seeded within 3D ECMs (Ctrl) and after being seeded at cell densities of 1×10⁵ cells/ml within 3D ECMs polymerized at 0.5 mg/ml and 2.0 mg/ml PSC. Note the shift in the colony forming potential for the cells seeded under the different conditions. These data include colonies that contained at least 2 cells (within each group represented on the abscissa; the left bar=control, middle bar=0.5 mg/ml PSC, right bar=2 mg/ml PSC).

FIG. 10 shows a histogram of the colony size formed (t=14 days) by an EPC population before being seeded within 3D ECMs (Ctrl) and after being seeded at cell densities of 1×10⁵ cells/ml within 3D ECMs polymerized at 0.5 mg/ml and 2.0 mg/ml PSC. Note the shift in the colony forming potential for the cells seeded under the different conditions. These data include single cell events. Note that EPCs grown within PSC show increased colony forming potential. (within each group represented on the abscissa; the left bar=control, middle bar=0.5 mg/ml PSC, right bar=2 mg/ml PSC).

FIG. 11 shows a histogram of the colony size formed by an EPC population before being seeded within 3D ECMs (Ctrl) and after being seeded at cell densities of 1×10⁵, 5×10⁵, and 1×10⁶ cells/ml within BD ECMs (1.5 mg/ml type I collagen+1 μg/ml fibronectin) or PSC ECMs (1.5 mg/ml pig skin type I collagen). Note the shift in the colony forming potential for the cells seeded under the different conditions. Note that EPCs grown within PSC show increased colony forming potential even at low seeding densities. (bars within each group (left to right) correspond to position in legend (top to bottom).

FIG. 12 shows the percentage of EPCs that underwent at least one cell division before being seeded within 3D ECMs (Ctrl) and after being seeded at cell densities of 1×10⁵, 5×10⁵, and 1×10⁶ cells/ml within BD ECMs (1.5 mg/ml type I collagen+1 μg/ml fibronectin) or PSC ECMs (1.5 mg/ml pig skin type I collagen). Note the increase in the percentage of dividing cells that was obtained after EPCs were seeded within 3D ECMs. Upon comparison of EPCs grown within BD and PSC ECM formulations, it was observed that EPCs seeded at a given cell density showed the greatest proliferative potential within the PSC formulation. (bars within each group (left to right) correspond to position in legend (top to bottom).

FIG. 13 shows an example of a microvessel network formed by endothelial colony-forming cells (ECFCs) seeded within engineered ECM prepared from pig skin collagen. ECFCs (bright white) were labeled with FITC conjugated UEA-1 lectin and collagen fibril microstructure was simultaneously visualized using 488 nm reflected light. Panel A illustrates both cellular and collagen fibril components of the construct. Panel B illustrates only the cellular component.

FIG. 14 shows ECFCs having formed endothelial-lined microvessels containing round, viable cells.

FIG. 15 shows 3D images demonstrating the differences in the vascular network development by ECFCs (1×10⁵ cells/ml) after 8 days within engineered ECMs prepared with pig skin collagen concentration, fibril volume fraction, and stiffness (G′) of (Panel A) 2 mg/ml, 38%, and 767 Pa and (Panel B) 0.5 mg/ml, 9%, and 48 Pa. Panels C and D represent an extensive vascular network produced by ECFCs after 14 days of culture within an engineered ECM. Panel C shows the network of ECFCs and Panel D provides a volume slice clearly demonstrating the lumens present in the vascular network. ECFCs (bright white) were labeled with FITC conjugated UEA-1 lectin and collagen fibril microstructure was simultaneously visualized using 488 nm reflected light (arrows denote visible lumens). Major tick mark on all images equals 50 μm.

FIG. 16 shows the shear storage modulus, or stiffness, over a range of collagen concentrations for pig skin compared to rat tail collagen (Panel A). The pig skin collagen demonstrated a broader range for shear storage modulus than the rat tail collagen over the range of collagen concentrations measured. Panel B shows the shear storage modulus over the same range of collagen concentrations. Again, the pig skin collagen demonstrated a broader range of shear storage modulus. Panel C depicts delta, which is the phase shift of the strain and stress waves over the range of collagen concentrations. The rat tail collagen was found to have a higher delta, and thus a more viscous response.

FIG. 17 shows the representative 2D projections of confocal reflection image stacks comparing the fibril microstructure for engineered ECMs prepared using commercial (Panels A and B) and pig skin (Panels C and D) collagen sources. Self-assembly conditions of both collagen sources were adjusted to yield engineered ECMs with the same fibril volume fraction (Panels A and C) or storage modulus (G′, stiffness; Panels B and D). Initial collagen concentration, G′, and fibril volume fraction data are provided.

FIG. 18 shows the mechanical properties of the 3D ECMs from type I pig skin collagen (PSC) and rat tail collagen (RTC): Panel A shows shear storage modulus (G′) of RTC and PSC ECMs versus collagen concentration; Panel B shows shear loss modulus (G″) of RTC and PSC ECMs versus collagen concentration; Panel C shows compressive modulus of RTC and PSC ECMs versus collagen concentration; and Panel D shows shear storage modulus (G′) versus fibril density for RTC and PSC ECMs. Values shown are the mean±standard deviation.

FIG. 19 shows the time course of vascular network formation.

FIG. 20 shows the vascular structure complexity over varying stiffness and cell seeding density in the pig skin collagen construct.

FIG. 21 shows brightfield images of ECMs from rat tail collagen (RTC) (Panels A and B) and pig skin collagen (PSC) (Panels C and D). Stiffnesses are shown in Pascals (Pa).

FIG. 22 shows a brightfield image of a vessel network formed by ECFCs cultured within a 3D collagen matrix. Distinct cellular phenotypes are noted as rounded cells (black arrows) found within the lumen of an endothelial lined vessel network (white arrows). Scale bar=100 μm.

FIG. 23 shows the modulation of cell surface marker expression for ECFCs cultured in vitro (6 days) within collagen matrices of varied fibril density and stiffness compared to the initial ECFC population (Control) (Panel A). Panel B shows the modulation of colony forming potential for ECFCs cultured in vitro (6 days) within collagen matrices at different seeding densities compared to the initial ECFC population (Control).

FIG. 24 shows ECM direct ECFC vessel formation in vivo. Panel A shows a photomicrograph (original magnification, ×20) of cellularized ECMs and surrounding mouse tissue. The two panels show consecutive sections of the same ECM stained with anti-mouse CD31 (mCD31) and anti-human CD31 (hCD31) to identify either mouse or human vessels respectively. Panel B shows a photomicrograph (original magnification, ×100) of ECFC vessels stained with hCD31. ECFC vessels and capillaries in the ECM are perfused with mouse red blood cells (arrows).

FIG. 25 shows histological cross-sections showing matrix-dependent ECFC response 2 weeks following subcutaneous implantation within NOD/SCID mice. ECFCs were implanted within collagen matrices that varied in fibril density and stiffness: Panel A=12% and 30 Pa (0.5 mg/ml) and Panel B=21% and 650 Pa (2.5 mg/ml). Functional vessels are indicated by arrows. Scale bar=50 μm.

FIG. 26 shows the characterization of 3D collagen matrix physical properties varied by collagen concentration. Fibril density increased linearly with collagen concentration as shown in 3D confocal reflection microscopy (CRM) images of 0.5 (Panel A) and 2.5 (Panel B) mg/ml matrices (scale bar=10 μm). Collagen matrix stiffness, indicated by G′ (Panel C, lines=linear regression trend lines), increased with increasing collagen concentration (p<0.05 between concentrations, n=3-5 for mechanical analyses) and with the addition of ECFCs (2×10⁶ cells/ml, gray lines, p<0.05 at each concentration except 3.5 mg/ml). Matrix fluid-like behavior, indicated by 6 (Panel D), was significantly affected by collagen concentration (* denotes p<0.05 within cell groups) and ECFC addition (p<0.05 at each concentration except 1.5 mg/ml). Matrix E, (Panel E) similarly increased with collagen concentration (* denotes p<0.05 within cell groups), but did not change significantly with ECFC addition (p>0.05 at all concentrations).

FIG. 27 shows immunohistochemical analysis of explanted ECFC matrices. Matrices, removed after 14 days, remodeled to a different extent dependent on collagen concentration. Representative sections (of n>6 implants in different mice) are shown for 0.5 (Panels B,C), 1.5 (Panels C,H), 2.5 (Panels D,I), and 3.5 (Panels E,J) mg/ml matrices (scale bars Panels A-E=1 mm, F-J=250 μm). Lower concentration matrices contracted to a greater degree than higher concentration matrices (Panels A-D). All matrices, except no cell controls (0.5 mg/ml Panels A,F), were able to direct ECFCs to form functional hCD31⁺ blood vessels which contained RBCs (Panels E-H, arrows=RBCs, arrow heads=hCD3130 vessels).

FIG. 28 shows quantification of RBC-containing vessel density within explanted ECFC matrices. Average total vessels per area (Panel A) and hCD31⁺ vessels per area (Panel B) decreased with increasing collagen concentration (* denotes p<0.05 between groups). The percentage of hCD31⁺ vessels increased with increasing concentration (Panel C).

FIG. 29 shows the analysis of RBC-containing vessel areas within explanted ECFC matrices. Average CD31+ vessel area (Panel A), total CD31+ vascular area (Panel B), and distribution of CD31+ vessel areas (Panel C) measured from histology images of vessels (* denotes p<0.05 between groups). Microscopy images show representative hCD31⁺ vessels with different areas between 51 and 100 μm² (Panel D), between 501 and 1000 μm² (Panel E), between 1001 and 2000 μm² (Panel F), and greater than 4000 μm² (Panel G, scale bar D-G=100 μm). Vessel morphology was significantly altered by matrix collagen concentration, with increasing concentration shifting towards larger average and total vessel areas.

FIG. 30 shows that the selective polymerization of PSC (Lanes 1 and 4) in the presence of glycerol resulted in oligomer- (Lanes 2 and 5) and monomer-rich fractions (Lanes 3 and 6) with different molecular compositions as determined by SDS-PAGE (8%; Lanes 1-3) and Western blot (Lanes 4-6) analyses. Unlike the monomer-rich fraction, PSC starting material and the oligomer-rich fraction showed a prominent protein band intermediate in molecular weight to β and γ bands (Oligo260) as well as several HMW bands (arrows). Western blot analysis confirmed that these components contained the type I collagen epitope.

FIG. 31 shows concentration dependent polymerization kinetics measured from the sigmoidal graph (A), including polymerization half-time (B), lag time (C), and polymerization rate during growth phase (D), for monomer- (open shapes, dashed lines) and oligomer-rich (solid shapes and lines) fractions as measured spectrophotometrically. Both single (squares) and batched (circles) PSC sources were polymerized using identical reaction conditions and collagen concentrations of 0.5, 0.7, and 1 mg/ml. Data points represent mean±SD (n≧4) with associated best-fit lines. Oligomer-rich fractions displayed the shortest polymerization half-time, shortest lag times, and fastest growth rates at each concentration tested (p<0.05).

FIG. 32 shows the shear storage modulus (G′,A), phase shift (δ, B), and compressive modulus (E_{c},C) for monomer- (open shapes, dashed lines) and oligomer-rich (solid shapes and lines) fractions prepared from single (squares) and batched (circles) PSC sources. Data points represent mean±SD (n≧4) with associated best-fit lines. The concentration dependence of measured parameters for the two fractions were statistically different (p<0.05), with the oligomer fraction displaying the greatest G′, lowest δ, and greatest E_{c} at each concentration tested (p<0.05).

FIG. 33 shows polymerization kinetic parameters, including polymerization half-time (A), lag time (B), and polymerization rate during growth phase (C), as measured spectrophotometrically for collagen formulations with AMW (average polymer molecular weight) ranging from 282 kDa to 603 kDa. Both single (squares) and batched (circles) PSC sources polymerized using identical reaction conditions and a collagen concentration of 0.7 mg/ml. Data points represent mean±SD (n≧4) with associated best-fit lines. Polymerization half-time and lag time decreased while growth rate increased with increasing AMW.

FIG. 34 shows that projected poresize decreased as AMW was increased from 288 kDa to 603 kDa as demonstrated by CRM. All matrices were polymerizaed at the same collagen concentration (0.7 mg/ml) and under the same conditions. 2D projections represent a total image thickness of 10 μm (101 slices, scale bar=10 μm).

FIG. 35 shows hierarchical matrix assembly for an oligomer-rich collagen formulation. Fibril intermediates were negatively stained and observed by TEM early during the assembly process. Fibrils appeared to grow longitudinally via microfibril condensation at tapered ends (black arrows). Lateral associations between growing segments contributed to banded fibrils with obvious and numerous branch points. Scale bar=500 nm.

FIG. 36 shows shear storage modulus (G′,A), phase shift (δ,B), and compressive modulus (E_{c},C) for matrices prepared with collagen AMW ranging from 282 kDa to 603 kDa. Both single (squares) and batched (circles) source formulations were polymerized using identical reaction conditions and a collagen concentration of 0.7 mg/ml. Data points represent mean±SD (n≧4). G′ and E_{c} increased linearly while δ decreased with increasing AMW.

FIG. 37 shows similar trends in polymerization half-time (A), fibril density (B), and viscoelastic properties (C-E) as a function of AMW were observed with monomer-and oligomer-rich fraction generated by differential salt precipitation of PSC. Data points represent mean±SD (n≦3).

FIG. 38 shows that tuning of the AMW or monomer/oligomer content of 3D collagen matrices directed different patterns of vacuole and lumen formation by cultured ECFCs. ECFCs (5×10⁵ cells/ml) were cultured for 2 days within matrices prepared with monomer- (left panels) or oligomer-rich (right panels) collagen formulations. Matrices were matched in terms of collagen concentration (1 mg/ml, upper panels) or G′ (200 Pa, lower panels). Scale bar=500 μm.

FIG. 39 shows the matching of concentration and stiffness for matrices prepared with monomer- or oligomer-rich collagen formulations. Matrices were matched in terms of 1 mg/ml collagen concentration [97.28 Pa (oligomer); 29.47 Pa (monomer)], 200 Pa stiffness [1.6 mg/ml (oligomer); 2.6 mg/ml (monomer)], or 375 Pa stiffness [2.2 mg/ml (oligomer); 3.7 mg/ml (monomer)].

FIG. 40 shows ECFCs (5×10⁵ cells/ml) cultured for 1 day (Panel A), 2 days (Panel B), or 3 days (Panel C) within matrices prepared with monomer- (top panels) or oligomer-rich (bottom panels) collagen formulations. Matrices were matched in terms of collagen concentration (1 mg/ml, left panels), G′ (200 Pa, middle panels), or G′ (375 Pa, right panels). Scale bar=500 μm FIG. 41 shows confocal analysis of ECFC morphology within 3D collagen matrices with varied physical properties after 18 hours of in vitro culture and immediately prior to subcutaneous implantation. Cellularized matrices were labeled with DAPI, and UEA-1 Lectin for visualization of nuclei and ECFCs, respectively. ECFC showed cord like networks but no apparent lumen formation in any collagen matrix formulation at this time point. While low concentration matrices (Panel A, 0.5 mg/ml) appear to have a lower cell density compared to higher concentration matrices (Panel B, 3.5 mg/ml, Scale bar=100 μm) the cell number is not different between the two conditions.

FIG. 42 shows the parabolic response of average human vessel area with shear storage modulus. The legend shows collagen concentration (mg/ml) for RTC (rat tail collagen) or PSC (pig skin collagen). The bars (left to right) match the legend identifiers (top to bottom).

FIG. 43 shows the quantitative analysis of the vessel area arranged as a distribution related to collagen concentration of rat tail (RTC) versus pig skin collagen (PSC). The legend shows collagen concentration (mg/ml) for RTC (rat tail collagen) or PSC (pig skin collagen). The bars (left to right) match the legend identifiers (top to bottom).

FIG. 44 shows the parabolic response of total vessel area with shear storage modulus in rat tail collagen (RTC) versus pig skin (PSC). Panel A: Total vascular area (mm²); Panel B: Total vessel area (mm²). For Panel A, the legend shows collagen concentration (mg/ml) for RTC (rat tail collagen) or PSC (pig skin collagen). The bars (left to right) match the legend identifiers (top to bottom).

FIG. 45 shows the matching of concentration and stiffness for matrices prepared with monomer- or oligomer-rich pig skin collagen formulations.

FIG. 46 shows the in vitro formation of cytoplasmic vacuoles in ECFCs implanted in pig skin collagen gels.

FIG. 47 shows vacuolar structures in collagen matrices. Scale bar=100 μm.

FIG. 48 shows analysis of ECFC morphology within 3D collagen matrices with varied physical properties. Cellularized matrices were labeled with DAPI, and UEA-1 Lectin for visualization of nuclei and ECFCs, respectively. Scale bar=50 μm.

FIG. 49 shows the analysis of vacuole density (Panel A), average vacuole area (Panel B), and total vacuole area (Panel C). Bar “A”=monomer (low collagen concentration, 1 mg/ml); Bar “B”=oligomer (low collagen concentration, 1 mg/ml); Bar “C”=monomer (high collagen concentration, 2.75 mg/ml); Bar “D”=oligomer (high collagen concentration, 2.75 mg/ml).

### DESCRIPTION OF THE ILLUSTRATIVE EMBODIMENTS

Methods and compositions for the support and differentiation of stem cells and for the formation of blood vessels and vascularized graft constructs are described. Applicants have developed and describe herein clinically-useful delivery strategies for rapid and effective vascularization of damaged or diseased tissues. The engineered collagen-based matrices as herein described are useful, for example, as 1) 3-dimensional culture systems for expansion of stem/progenitor cells, 2) clinically relevant delivery vehicles for cell-based therapies, and 3) engineered tissue constructs with preformed vascular networks or enhanced capability for forming vascular networks in vivo.

As used herein “engineered purified collagen-based matrix” means a purified collagen-based matrix that is polymerized in vitro under predetermined conditions selected from the group consisting of, but not limited to, pH, phosphate concentration, temperature, buffer composition, ionic strength, and composition and concentration of the collagen. “Engineering a matrix” means polymerizing an “engineered purified collagen-based matrix” in vitro. An “engineered purified collagen-based matrix” can be made from purified collagen or partially purified ECM components.

In one embodiment, a method of regulating vessel density within an engineered purified collagen-based matrix composition is described. In another embodiment, a method of regulating vessel area within an engineered purified collagen-based matrix composition is described. The methods described herein comprise the steps of engineering the purified collagen-based matrix comprising collagen fibrils, and contacting the matrix with endothelial progenitor cells wherein said contacting results in vessel formation and an increase in vessel density and/or vessel size within the matrix with varying collagen concentrations. In yet another embodiment, the vessel density and/or vessel size is increased within the engineered purified collagen-based matrix composition prior to implantation of the graft composition. In addition, all of the embodiments described below apply to any embodiment described in the summary section of this application.

In any embodiment described herein, the engineered, purified collagen-based matrices are prepared by utilizing acid-solubilized type I collagen and defined polymerization (self-assembly) conditions that are controlled to yield 3-dimensional collagen extracellular matrices (ECMs) with a broad range of controlled assembly kinetics (e.g. polymerization half-time), molecular compositions, and fibril microstructure-mechanical properties, for example, as described in U.S. patent application Ser. Nos. 11/435,635 (published Nov. 22, 2007, as Publication No. 2007-0269476 A1) and 11/903,326 (published Oct. 30, 2008, as Publication No. 2008-0268052), each incorporated herein by reference.

Purified collagen can be obtained from a number of sources, including for example, porcine skin, to construct the engineered, purified collagen-based matrices described herein. Suitable tissues useful as a collagen-containing source material for isolating collagen to make the engineered, purified collagen-based matrices described herein are submucosa tissues or any other extracellular matrix-containing tissues of a warm-blooded vertebrate. Suitable methods of preparing submucosa tissues are described in U.S. Pat. Nos. 4,902,508; 5,281,422; and 5,275,826, each incorporated herein by reference. Extracellular matrix material-containing tissues other than submucosa tissue may be used in accordance with the methods and compositions described herein. Methods of preparing other extracellular matrix material-derived tissues are known to those skilled in the art. For example, see U.S. Pat. Nos. 5,163,955 (pericardial tissue); 5,554,389 (urinary bladder submucosa tissue); 6,099,567 (stomach submucosa tissue); 6,576,265 (extracellular matrix tissues generally); 6,793,939 (liver basement membrane tissues); and U.S. patent application publication no. US-2005-0019419-A1 (liver basement membrane tissues); and international publication no. WO 2001/45765 (extracellular matrix tissues generally), each incorporated herein by reference. In various other embodiments, the collagen-containing source material can be selected from the group consisting of placental tissue, ovarian tissue, uterine tissue, animal tail tissue, and skin tissue. Any suitable extracellular matrix-containing tissue can be used as a collagen-containing source material.

An illustrative preparation method for preparing submucosa tissues as a source of collagen is described in U.S. Pat. No. 4,902,508, the disclosure of which is incorporated herein by reference. In one embodiment, a segment of vertebrate intestine, for example, preferably harvested from porcine, ovine or bovine species, but not excluding other species, is subjected to abrasion using a longitudinal wiping motion to remove cells or to cell-removal by hypotonic or hypertonic lysis. In this embodiment, the submucosa tissue is rinsed under hypotonic conditions, such as with water or with saline under hypotonic conditions and is optionally sterilized. In another illustrative embodiment, such compositions can be prepared by mechanically removing the luminal portion of the tunica mucosa and the external muscle layers and/or lysing resident cells with hypotonic or hypertonic washes, such as with water or saline. In these embodiments, the submucosa tissue can be stored in a hydrated or dehydrated state prior to extraction. In various aspects, the submucosa tissue can comprise any delamination embodiment, including the tunica submucosa delaminated from both the tunica muscularis and at least the luminal portion of the tunica mucosa of a warm-blooded vertebrate.

In the various embodiments described herein, the purified collagen can also contain glycoproteins, proteoglycans, glycosaminoglycans (e.g., chondroitins and heparins), etc. extracted from the insoluble fraction with the collagen. The engineered, purified collagen-based matrices prepared by the methods described herein can serve as matrices for the regrowth of endogenous tissues at the implantation site (e.g., biological remodeling) which can assume the features of the tissue(s) with which they are associated at the site of implantation, insertion, or injection.

In the various illustrative embodiments described herein, the collagen matrices, including an engineered purified collagen-based matrix, can be disinfected and/or sterilized using conventional sterilization techniques including glutaraldehyde tanning, formaldehyde tanning at acidic pH, propylene oxide or ethylene oxide treatment, gas plasma sterilization, gamma radiation, electron beam, and/or peracetic acid sterilization. Sterilization techniques which do not adversely affect the structure and biotropic properties of the collagen can be used. Illustrative sterilization techniques are exposing the collagen-containing source material, the purified collagen, or the engineered purified collagen-based matrix to peracetic acid, 1-4 Mrads gamma irradiation (or 1-2.5 Mrads of gamma irradiation), ethylene oxide treatment, or gas plasma sterilization. In one embodiment, the collagen-containing source material, the purified collagen, or the engineered purified collagen-based matrix can be subjected to one or more sterilization processes. In an illustrative embodiment, peracetic acid can be used for sterilization.

Typically, prior to extraction, the collagen-containing source material is comminuted by tearing, cutting, grinding, or shearing the collagen-containing source material. In one illustrative embodiment, the collagen-containing source material can be comminuted by shearing in a high-speed blender, or by grinding the collagen-containing source material in a frozen state (e.g., at a temperature of −20° C., −40° C., −60° C., or −80° C. or below prior to or during the comminuting step) and then lyophilizing the material to produce a powder having particles ranging in size from about 0.1 mm² to about 1.0 mm². In one illustrative embodiment, the collagen-containing source material is comminuted by freezing and pulverizing under liquid nitrogen in an industrial blender. In this embodiment, the collagen-containing source material can be frozen in liquid nitrogen prior to, during, or prior to and during the comminuting step.

In any of the illustrative embodiments described herein, after comminuting the collagen-containing source material, the material is mixed (e.g., by blending or stirring) with an extraction solution to extract and remove soluble proteins. Illustrative extraction solutions include sodium acetate (e.g., 0.5 M and 1.0 M). Other methods for extracting soluble proteins are known to those skilled in the art and are described in detail in U.S. Pat. No. 6,375,989, incorporated herein by reference. Illustrative extraction excipients include, for example, chaotropic agents such as urea, guanidine, sodium chloride or other neutral salt solutions, magnesium chloride, and non-ionic or ionic surfactants.

In any illustrative aspect described herein, after the initial extraction, the soluble fraction can be separated from the insoluble fraction to obtain the insoluble fraction. For example, the insoluble fraction can be separated from the soluble fraction by centrifugation (e.g., 2000 rpm at 4° C. for 1 hour). In alternative embodiments, other separation techniques known to those skilled in the art, such as filtration, can be used. In one embodiment, the initial extraction step can be repeated one or more times, discarding the soluble fractions. In another embodiment, after completing the extractions, one or more steps can be performed of washing with water the insoluble fraction, followed by centrifugation, and discarding of the supernatant where the water is the supernatant.

In any of the embodiments described herein, the insoluble fraction can then be extracted (e.g., with 0.075 M sodium citrate) to obtain the isolated collagen. In illustrative aspects the extraction step can be repeated multiple times retaining the soluble fractions. In one embodiment, the accumulated soluble fractions can be combined and can be clarified to form the soluble fraction, for example by centrifugation (e.g., 2000 rpm at 4° C. for 1 hour).

In any embodiment described herein, the soluble fraction can be fractionated to precipitate the isolated collagen. In one illustrative aspect, the soluble fraction can be fractionated by dialysis. Suitable molecular weight cut-offs for the dialysis tubing or membrane are from about 3,500 to about 12,000 or about 3,500 to about 5,000 or about 12,000 to about 14,000. In various illustrative embodiments, the fractionation, for example by dialysis, can be performed at about 2° C. to about 37° C. for about 1 hour to about 96 hours. In one embodiment, the soluble fraction is dialyzed against a buffered solution (e.g., 0.02 M sodium phosphate dibasic). However, the fractionation can be performed at any temperature, for any length of time, and against any suitable buffered solution. In one embodiment, the precipitated collagen is then collected by centrifugation (e.g., 2000 rpm at 4° C. for 1 hour). In another embodiment, after precipitation, one or more steps can be performed of washing the precipitate with water, followed by centrifugation, and discarding the supernatant where the water is the supernatant.

In any of the embodiments described herein, the precipitated collagen can then be resuspended in an aqueous solution wherein the aqueous solution is acidic. For example, the aqueous acidic solution can be an acetic acid solution, but any other acids including hydrochloric acid, formic acid, lactic acid, citric acid, sulfuric acid, ethanoic acid, carbonic acid, nitric acid, or phosphoric acid can be used. For example, acids, at concentrations of from about 0.001 N to about 0.1 N, from about 0.005 N to about 0.1 N, from about 0.01 N to about 0.1 N, from about 0.05 N to about 0.1 N, from about 0.001 N to about 0.05 N, from about 0.001 N to about 0.01 N, or from about 0.01 N to about 0.05 N can be used to resuspend the precipitate.

The term “lyophilized” means that water is removed from the composition, typically by freeze-drying under a vacuum. In one illustrative aspect, the isolated resuspended collagen can be lyophilized after it is resuspended. In another illustrative embodiment, the engineered purified collagen-based matrix itself can be lyophilized. In one illustrative lyophilization embodiment, the resuspended collagen is first frozen, and then placed under a vacuum. In another lyophilization embodiment, the resuspended collagen can be freeze-dried under a vacuum. In another lyophilization embodiment, the precipitated collagen can be lyophilized before resuspension. Any method of lyophilization known to the skilled artisan can be used.

In any of the embodiments described herein, the acids described above can be used as adjuvants for storage after lyophilization in any combination. The acids that can be used as adjuvants for storage include hydrochloric acid, acetic acid, formic acid, lactic acid, citric acid, sulfuric acid, ethanoic acid, carbonic acid, nitric acid, or phosphoric acid, and these acids can be used at any of the above-described concentrations. In one illustrative embodiment, the lyophilizate can be stored (e.g., lyophilized in and stored in) an acid, such as acetic acid, at a concentration of from about 0.001 N to about 0.5 N or from about 0.01 N to about 0.5 N. In another embodiment, the lyophilizate can be stored in water with a pH of about 6 or below. In another embodiment, the lyophilized product can be stored dry. In other illustrative embodiments, lyoprotectants, cryoprotectants, lyophilization accelerators, or crystallizing excipients (e.g., ethanol, isopropanol, mannitol, trehalose, maltose, sucrose, tert-butanol, and tween 20), or combinations thereof, and the like can be present during lyophilization.

In any of the embodiments described herein, the resuspended collagen is sterilized. Suitable sterilizing and/or disinfecting agents are described above, but any sterilizing and/or disinfecting agent or method of sterilization known in the art can be used. The resuspended collagen can be sterilized using chloroform, glutaraldehyde, formaldehyde, acidic pH, propylene oxide, ethylene oxide, gas plasma sterilization, gamma radiation, electron beam sterilization, or peracetic acid sterilization, or combinations thereof, and the like. Illustrative sterilization techniques are exposing the resuspended collagen to peracetic acid, 1-4 Mrads gamma irradiation (or 1-2.5 Mrads of gamma irradiation), ethylene oxide treatment, or gas plasma sterilization.

In any embodiment described herein, the isolated collagen can be sterilized before lyophilization. In another illustrative embodiment the isolated collagen can be sterilized after lyophilization or the collagen-containing source material can be sterilized. Sterilization of the collagen-containing source material can be performed, for example, as described in U.S. Pat. Nos. 4,902,508 and 6,206,931, incorporated herein by reference. In another illustrative embodiment, the engineered purified collagen-based matrix is sterilized.

In any of the illustrative embodiments described herein, the purified collagen is directly sterilized after resuspension, for example, with peracetic acid or with peracetic acid and ethanol (e.g., by the addition of 0.18% peracetic acid and 4.8% ethanol to the resuspended collagen solution before lyophilization). In another embodiment, sterilization can be carried out during the fractionation step. For example, the isolated collagen composition can be dialyzed against chloroform, peracetic acid, or a solution of peracetic acid and ethanol to disinfect or sterilize the isolated collagen. Illustratively, the isolated collagen can be sterilized by dialysis against a solution of peracetic acid and ethanol (e.g., 0.18% peracetic acid and 4.8% ethanol). The chloroform, peracetic acid, or peracetic acid/ethanol can be removed prior to lyophilization, for example by dialysis against an acid, such as 0.01 N acetic acid. In an alternative embodiment, the lyophilized composition can be sterilized directly after rehydration, for example, by the addition of 0.18% peracetic acid and 4.8% ethanol. In this embodiment, the sterilizing agent can be removed prior to polymerization of the purified collagen to form fibrils.

If the purified collagen or the engineered purified collagen-based matrix is lyophilized, the lyophilized composition can be stored frozen, refrigerated, or at room temperature (for example, at about −80° C. to about 25° C.). Storage temperatures are selected to stabilize the collagen. The compositions can be stored for about 1-26 weeks, or longer.

In any embodiment described herein, the purified collagen can be dialyzed against 0.01 N acetic acid, for example, prior to lyophilization to remove the sterilization solution and so that the purified collagen is in a 0.01 N acetic acid solution. In another embodiment, the purified collagen can be dialyzed against hydrochloric acid, for example, prior to lyophilization and can be lyophilized in hydrochloric acid and redissolved in hydrochloric acid, acetic acid, or water.

If the purified collagen is lyophilized, the resulting lyophilizate can be redissolved in any solution, but may be redissolved in an acidic solution or water. In various aspects, the lyophilizate can be redissolved in, for example, acetic acid, hydrochloric acid, formic acid, lactic acid, citric acid, sulfuric acid, ethanoic acid, carbonic acid, nitric acid, or phosphoric acid, at any of the above-described concentrations, or can be redissolved in water. In one illustrative embodiment the lyophilizate is redissolved in 0.01 N acetic acid.

For use in producing engineered purified collagen-based matrix that can be injected or implanted in vivo or used for other purposes in vitro, the redissolved lyophilizate can be subjected to varying conditions (e.g., pH, phosphate concentration, temperature, buffer composition, ionic strength, and composition and concentration of the purified collagen components (dry weight/ml)) that result in polymerization to form an engineered purified collagen-based matrix with specific characteristics.

In any of the illustrative embodiments described herein, as discussed above, the polymerization reaction for the engineered purified collagen-based matrices can be conducted in a buffered solution using any biologically compatible buffer system known to those skilled in the art. For example, the buffer may be selected from the group consisting of phosphate buffer saline (PBS), Tris (hydroxymethyl)aminomethane Hydrochloride (Tris-HCl), 3-(N-Morpholino) Propanesulfonic Acid (MOPS), piperazine-n,n′-bis(2-ethanesulfonic acid) (PIPES), [n-(2-Acetamido)]-2-Aminoethanesulfonic Acid (ACES), N-[2-hydroxyethyl]piperazine-N′-[2-ethanesulfonic acid] (HEPES) and 1,3-bis[tris (Hydroxymethyl)methylamino]propane (Bis Tris Propane). In one embodiment the buffer is PBS, Tris, or MOPS and in one embodiment the buffer system is PBS, and more particularly 10×PBS. In accordance with one embodiment, the 10×PBS buffer at pH 7.4 comprises the following ingredients:

1.37 M NaCl

0.027 M KCl

0.081 M Na₂HPO₄

0.015 M KH₂PO₄

5 mM MgCl₂

55.5 mM glucose

All of the conditions that can be varied to polymerize and engineer the purified collagen-based matrices described herein (e.g., pH, phosphate concentration, temperature, buffer composition, ionic strength, and composition and concentration of the purified collagen components (dry weight/ml)) are described in U.S. application Ser. No. 11/903,326 (published Oct. 30, 2008, as Publication No. 2008-0268052), incorporated herein by reference. Tissue graft constructs can be formed from the engineered, purified collagen-based matrices described herein and can be injected or implanted, or, for example, applied topically to wounds, all by methods known to those skilled in the art.

The purified collagen is derived from a collagen-containing source material and, in some embodiments, may contain glycoproteins, such as laminin and fibronectin, proteoglycans, such as serglycin, versican, decorin, and perlecan, and glycosaminoglycans. In one embodiment, the purified collagen can be further purified or partially purified and the purified or partially purified composition can be used in accordance with the methods described herein or mixtures of partially purified or purified components can be used. As used herein, the term “purified” means the isolation of collagen in a form that is substantially free from other components (e.g., typically the total amount of other components present in the composition represents less than 5%, or more typically less than 0.1%, of total dry weight).

As discussed, the engineered purified collagen-based matrices as herein described may be made under controlled conditions to obtain particular mechanical properties. For example, the engineered purified collagen-based matrices described may have desired collagen fibril density, pore size (fibril-fibril branching), elastic modulus, tensile strain, tensile stress, linear modulus, compressive modulus, loss modulus, fibril area fraction, fibril volume fraction, collagen concentration, cell seeding density, shear storage modulus (G′ or elastic (solid-like) behavior), and phase angle delta (δ or the measure of the fluid (viscous)- to solid (elastic) -like behavior; δ equals 0° for Hookean solid and 90° for Newtonian fluid).

As used herein, a “modulus” can be an elastic or linear modulus (defined by the slope of the linear region of the stress-strain curve obtained using conventional mechanical testing protocols; i.e., stiffness), a compressive modulus, a loss modulus, or a shear storage modulus (e.g., a storage modulus). These terms are well-known to those skilled in the art.

As used herein, a “fibril volume fraction” is defined as the percent area of the total area occupied by fibrils in 3 dimensions.

As used herein, tensile or compressive stress “σ” is the force carried per unit of area and is expressed by the equation:

$$σ = \frac{P}{A} = \frac{P}{ab}$$

where:
s=stress
P=force
A=cross-sectional area
a=width
h=height

The force (P) produces stresses normal (i.e., perpendicular) to the cross section of the part (e.g., if the stress tends to lengthen the part, it is called tensile stress, and if the stress tends to shorten the part, it is called compressive stress).

As used herein, “tensile strain” is the strain caused by bending and/or stretching a material.

In any embodiment described herein, the fibril volume fraction of the matrix is about 1% to about 60%. In various embodiments, the engineered purified collagen-based matrix can contain fibrils with specific characteristics, for example, a fibril volume fraction (i.e., density) of about 2% to about 60%, about 2% to about 40%, about 5% to about 60%, about 15% to about 60%, about 5% to about 40%, about 1% to about 50%, about 1% to about 40%, about 1% to about 30%, about 1% to about 20%, about 1% to about 15%, about 1% to about 10%, about 1% to about 5%, about 5% to about 20%, about 5% to about 50%, about 12% to about 20%, about 20% to about 60%, about 30% to about 50%, about 30% to about 60%, about 50% to about 60%, about 1% to about 2%, about 1% to about 3%, and about 1% to about 4%. In various illustrative embodiments, the fibril volume fraction is about 1%, about 5%, about 10%, about 15%, about 20%, about 25%, about 30%, about 40%, about 50%, or about 60%.

In any of the illustrative embodiments described herein, the engineered purified collagen-based matrix can contain fibrils with specific characteristics, including, but not limited to, a modulus (e.g., a compressive modulus, loss modulus, or a storage modulus) of about 1 Pa to about 75 Pa, about 10 Pa to about 700 Pa, about 2500 Pa to about 18,000 Pa, about 10 Pa to about 75 Pa, about 1 Pa to about 700 Pa, about 10 Pa to about 10,000 Pa, and about 1 Pa to about 18,000 Pa.

In any of the embodiments described herein, the engineered purified collagen-based matrix can contain fibrils with specific characteristics, including, but not limited to, a storage modulus of about 10 Pa to about 700 Pa. In another illustrative embodiment, the storage modulus of the matrix is about 10 Pa to about 600 Pa, about 1 Pa to about 15 Pa, about 25 Pa to about 50 Pa, about 10 Pa to about 500 Pa, about 10 Pa to about 250 Pa, about 40 Pa to about 50 Pa, about 50 Pa to about 700 Pa, about 50 Pa to about 500 Pa, about 100 Pa to about 700 Pa, about 100 Pa to about 500 Pa, about 100 Pa to about 250 Pa, about 200 Pa to about 700 Pa, about 500 Pa to about 700 Pa, and about 650 Pa to about 700 Pa.

In any of the embodiments described herein, the engineered purified collagen-based matrix can contain fibrils with specific characteristics, including, but not limited to, a loss modulus of about 1 Pa to about 75 Pa. In another illustrative embodiment, the loss modulus of the matrix is about 1 Pa to about 60 Pa, about 1 Pa to about 50 Pa, about 1 Pa to about 40 Pa, about 1 Pa to about 30 Pa, about 1 Pa to about 25 Pa, about 1 Pa to about 20 Pa, about 1 Pa to about 10 Pa, about 2 Pa to about 70, about 2 Pa to about 50 Pa, about 5 Pa to about 70 Pa, about 5 Pa to about 50 Pa, about 5 Pa to about 30 Pa, about 5 Pa to about 25 Pa, about 10 Pa to about 70 Pa, and about 10 Pa to about 50 Pa.

In any of the embodiments described herein, the engineered purified collagen-based matrix can contain fibrils with specific characteristics, including, but not limited to, a compressive modulus of about 2,500 Pa to about 18,000 Pa, about 10,000 Pa to about 120,000 Pa, 5,000 Pa to about 80,000 Pa, about 15,000 Pa to about 50,000 Pa, about 90,000 Pa to about 150,000 Pa, and about 15,000 Pa to about 140,000 Pa. In another illustrative embodiment, the compressive modulus of the matrix is about 2,500 to about 15,000, about 2,500 to about 10,000, about 5,000 to about 10,000, about 5,000 to about 12,000, about 5,000 to about 15,000, about 5,000 to about 18,000, about 15,000 Pa to about 50,000 Pa, about 10,000 Pa to about 50,000 Pa, about 15,000 Pa to about 60,000 Pa, about 100,000 Pa to about 150,000 Pa, and about 75,000 Pa to about 150,000 Pa.

In any of the embodiments described herein, the engineered purified collagen-based matrix can contain fibrils with specific characteristics, including, but not limited to, a phase angle delta (δ) of about 0° to about 12°, about 0° to about 5°, about 1° to about 5°, about 4° to about 12°, about 5° to about 7°, about 8° to about 10°, and about 5° to about 10°.

In any of the embodiments described herein, the composition comprises one or more vessels. In one embodiment, the blood vessels are produced de novo. In another embodiment, methods for promoting vessel formation within an engineered purified collagen-based matrix are described. In this embodiment, the method comprises the steps of engineering the purified collagen-based matrix comprising collagen fibrils, and contacting the matrix with endothelial progenitor cells, wherein one or more vessels are formed within the matrix. In another embodiment, the one or more vessels are isolated from the matrix. In yet another embodiment, the isolated one or more vessels are implanted into the tissue of a patient, using methods known in the art. The isolated vessels may be used for the treatment of various disease states as herein described. In another embodiment, a method of forming vessels in vivo or in vitro is provided. The method for in vivo formation of vessels comprises the step of implanting an engineered, purified collagen-based matrix comprising collagen fibrils and endothelial progenitor cells into a patient wherein vessel formation at the implantation site occurs in vivo.

In any of the embodiments described herein, a method for increasing the density of vessels within the engineered purified collagen-based matrix is described, wherein the vessel density is regulated by varying the mechanical properties of the matrix, for example, by varying the collagen concentration, matrix stiffness, and/or fibril density of the matrix. In various illustrative embodiments, the vessel density of the collagen-based matrix is about 40 vessels/mm²to about 80 vessels/mm², about 35 vessels/mm² to about 75 vessels/mm², about 40 vessels/mm² to about 75 vessels/mm², about 40 vessels/mm² to about 65 vessels/mm², about 45 vessels/mm² to about 65 vessels/mm², and about 50 vessels/mm² to about 65 vessels/mm². In further illustrative aspects, the vessel density of the collagen-based matrix is about 5 vessels/mm² to about 30 vessels/mm², about 10 vessels/mm² to about 30 vessels/mm², about 10 vessels/mm² to about 25 vessels/mm², about 10 vessels/mm² to about 20 vessels/mm², about 15 vessels/mm² to about 35 vessels/mm², about 10 vessels/mm² to about 35 vessels/mm², and about 10 vessels/mm² to about 90 vessels/mm².

In any of the embodiments described herein, a method for enhancing the area of vessels within the collagen-based matrix is described, wherein the vessel area is regulated by varying the mechanical properties of the engineered purified collagen-based matrix, for example, by varying the collagen concentration, matrix stiffness, and/or fibril density of the matrix. In various illustrative embodiments, the average vessel area within the collagen-based matrix is about 300 μm² to about 600 μm², about 300 μm² to about 500 μm², about 350 μm² to about 600 μm², about 350 μm² to about 500 μm², about 400 μm² to about 600 μm², about 400 μm² to about 500 μm², about 325 μm² to about 450 μm², about 350 μm² to about 450 μm², about 400 μm² to about 450 μm², about 100 μm² to about 250 μm², about 200 μm² to about 500 μm², and about 0 μm² to about 4000 μm². In any of the further illustrative aspects described herein, the average vessel size within the collagen-based matrix is about 50 μm² to about 300 μm², about 50 μm² to about 250 μm², about 100 μm² to about 300 μm², about 10 μm² to about 300 μm², about 10 μm² to about 200 μm², about 50 μm² to about 200 μm², about 100 μm² to about 200 μm², about 50 μm² to about 100 μm², and about 50 μm² to about 150 μm².

In any of the embodiments described herein, methods are described for the treatment of a patient. For example, a patient may be treated wherein the tissue of the patient is in need of vascularization. The method comprises the steps of engineering a purified collagen-based matrix comprising collagen fibrils, contacting the matrix with endothelial progenitor cells wherein vessels are formed de novo (e.g., either in vitro or in vivo), isolating the vessels from the matrix, and implanting the vessels into the tissue of the patient. Disease states or injuries that may be treated using the compositions and methods described herein include, for example, complications associated with diabetes, peripheral vascular disease, cerebral ischemia, cardiovascular disease (e.g. coronary artery disease), and for wound healing, including the treatment of wounds in a burn patient (e.g., to increase the rate of revascularization), treatment to reduce or prevent scarring and stricture formation, and the treatment of wounds in a diabetic patient (e.g., to treat limb ischemia or diabetic ulcers). In another embodiment, the vessels are not isolated from the matrix, but are implanted with the matrix. In yet another embodiment, the matrix is implanted and the vessels form in vivo.

In any of the embodiments described herein, the collagen can also contain glycoproteins, proteoglycans, glycosaminoglycans (e.g., chondroitins and heparins), etc. extracted from the insoluble fraction with the collagen. The engineered purified collagen-based matrices prepared by the methods described herein can serve as matrices for the regrowth of endogenous tissues at the implantation site (e.g., biological remodeling) which can assume the characterizing features of the tissue(s) with which they are associated at the site of implantation, insertion, or injection.

In any of the illustrative embodiments described herein, qualitative and quantitative microstructural characteristics of the engineered purified collagen-based matrices can be determined by environmental or cryostage scanning electron microscopy, transmission electron microscopy, confocal microscopy, second harmonic generation multi-photon microscopy. In another embodiment, polymerization kinetics may be determined by spectrophotometry or time-lapse confocal reflection microscopy. In another embodiment, tensile, compressive and viscoelastic properties can be determined by rheometry or tensile testing. In another embodiment, a rat subcutaneous injection model can be used to determine remodeling properties. All of these methods are known in the art or are further described in U.S. patent application Ser. No. 11/435,635 (published Nov. 22, 2007, as Publication No. 2007-0269476 A1), or are described in Roeder et al., J. Biomech. Eng., vol. 124, pp. 214-222 (2002), in Pizzo et al., J. Appl. Physiol., vol. 98, pp. 1-13 (2004), Fulzele et al., Eur. J. Pharm. Sci., vol. 20, pp. 53-61 (2003) , Griffey et al., J. Biomed. Mater. Res., vol. 58, pp. 10-15 (2001), Hunt et al., Am. J. Surg., vol. 114, pp. 302-307 (1967), and Schilling et al., Surgery, vol. 46, pp. 702-710 (1959), incorporated herein by reference.

Typically, the engineered purified collagen-based matrices are prepared from isolated collagen at collagen concentrations ranging from about 0.05 mg/ml to about 5.0 mg/ml, about 1.0 mg/ml to about 3.0 mg/ml, about 0.1 mg/ml to about 4.0 mg/ml, about 0.5 mg/ml to about 3.5 mg/ml, about 0.5 mg/ml to about 5.0 mg/ml, about 0.05 mg/ml to about 10 mg/ml, about 0.05 to about 20 mg/ml, about 0.05 mg/ml to about 3.0 mg/ml, about 0.3 to about 1 mg/ml, about 0.3 to about 1.5 mg/ml, about 0.3 mg/ml to about 5 mg/ml, about 0.75 mg/ml to about 5 mg/ml, about 1 mg/ml to about 5 mg/ml, about 1 mg/ml to about 2 mg/ml, about 1 mg/ml to about 3 mg/ml, about 1 mg/ml to about 4 mg/ml, about 1.5 mg/ml to about 5 mg/ml, and about 1.5 mg/ml to about 3 mg/ml. In various illustrative embodiments, the collagen concentration is about 0.3 mg/ml, about 0.5 mg/ml, about 0.75 mg/ml, about 1.0 mg/ml, about 1.5 mg/ml, about 2.0 mg/ml, about 2.5 mg/ml, about 3.0 mg/ml, about 3.5 mg/ml, or about 5.0 mg/ml.

In any of these embodiments the engineered graft construct may further comprise an added population of cells. The added population of cells may comprise one or more cell populations. In various embodiments, the cell populations comprise a population of mesodermally derived cells selected from the group consisting of endothelial cells, neural cells, pericytes, osteoblasts, fibroblasts, smooth muscle cells, skeletal muscle cells, cardiac muscle cells, mesenchymal cells, adipocytes, adipose stromal cells, progenitor cells (e.g., stem cells, including bone marrow progenitor cells), unrestricted somatic stem cells (USSCs), endothelial progenitor cells (EPCs), endothelial colony forming cells (ECFCs), and osteogenic cells. In various embodiments, the engineered purified collagen-based matrix can be seeded with one or more cell types in combination.

In any of the embodiments described herein, a source of cells suitable to form vascular networks are endothelial progenitor cells (EPCs). EPCs are released into the circulation of a patient and home to sites of vessel formation in both physiological and pathological settings. EPCs integrate into injured or disease sites including tumors, ischemic skeletal and cardiac muscle, and ulcers.

As used herein, “stem cell” refers to an unspecialized cell from an embryo, fetus, or adult that is capable of self-replication or self-renewal and can develop into specialized cell types of a variety of tissues and organs (i.e., potency). The term as used herein, unless further specified, encompasses totipotent cells (those cells having the capacity to differentiate into extra-embryonic membranes and tissues, the embryo, and all post-embryonic tissues and organs), pluripotent cells (those cells that can differentiate into cells derived from any of the three germ layers), multipotent cells (those cells having the capacity to differentiate into a limited range of differentiated cell types, e.g., mesenchymal stem cells, adipose-derived stem cells, endothelial stem cells, etc.), oligopotent cells (those cells that can differentiate into only a few cell types, e.g., lymphoid or myeloid stem cells), and unipotent cells (those cells that can differentiate into only one cell type, e.g., muscle stem cells). Stem cells may be isolated from, for example, circulating blood, umbilical cord blood, or bone marrow by methods well-known to those skilled in the art.

Examples of progenitor cells include those that give rise to blood cells, fibroblasts, endothelial cells, epithelial cells, smooth muscle cells, skeletal muscle cells, cardiac muscle cells, multi-potential progenitor cells, pericytes, and osteogenic cells. The population of progenitor cells can be selected based on the cell type of the intended tissue to be repaired. For example, if skin is to be repaired, the population of progenitor cells will give rise to non-keratinized epithelial cells or if cardiac tissue is to be repaired, the progenitor cells can produce cardiac muscle cells. The matrix composition can also be seeded with autogenous cells isolated from the patient to be treated. In an alternative embodiment the cells may be xenogeneic or allogeneic in nature.

In any of the embodiments described herein, the stem cells are seeded within the engineered purified collagen-based matrix at a cell density of about 1×10⁶ to about 1×10⁸ cells/ml, or at a density of about 1×10³ to about 2×10⁶ cells/ml. In one embodiment stem cells are seeded at a density of less than 5×10⁴ cells/ml, more typically at a density of about 5×10⁴ cells/ml. In another embodiment cells are seeded at a density of less than 1×10⁴ cells/ml. In another embodiment, cells are seeded at a density selected from a range of about 1×10² to about 5×10⁶, about 0.3×10⁴ to about 60×10⁴ cells/ml, and about 0.5×10⁴ to about 50×10⁴ cells/ml. In various illustrative embodiments, the cells are seeded at a density of about 0.3×10⁴ cells/ml, about 5×10⁴ cells/ml, about 10×10⁴ cells/ml, about 20×10⁴ cells/ml, about 40×10⁴ cells/ml, 60×10⁴ cells/ml, and 1×10⁵, about 5×10⁵, about 1×10⁶ cells/ml, and about 2×10⁶ cells/ml. The cells are maintained or differentiated according to methods described herein or to methods well-known to the skilled artisan for cell culture.

In any of the various embodiments described herein, the engineered purified collagen-based matrices of the present invention can be combined, prior to, during, or after polymerization, with nutrients, including minerals, amino acids, sugars, peptides, proteins, vitamins (such as ascorbic acid), or glycoproteins that facilitate cellular proliferation, such as laminin and fibronectin, hyaluronic acid, or growth factors such as epidermal growth factor, platelet-derived growth factor, transforming growth factor beta, or fibroblast growth factor, and glucocorticoids such as dexamethasone. In other illustrative embodiments, fibrillogenesis inhibitors, such as glycerol, glucose, or polyhydroxylated compounds can be added prior to or during polymerization. In accordance with one embodiment, cells can be added to the isolated collagen as the last step prior to the polymerization or after polymerization of the engineered purified collagen-based matrix. In other illustrative embodiments, cross-linking agents, such as carbodiimides, aldehydes, lysl-oxidase, N-hydroxysuccinimide esters, imidoesters, hydrazides, and maleimides, and the like can be added before, during, or after polymerization.

In any of the embodiments described herein, the cells are isolated from the matrix using an enzyme. For example, stem cells are isolated from the matrix using collagenase or a solution thereof. Additional enzymes useful for isolation of cells from the matrix include, for example, proteases such as serine proteases, thiol proteases, and metalloproteinases, including the matrix metalloproteinases such as the collagenases, gelatinases, stromelysins, and membrane type metalloproteinase, or combinations thereof.

In any of the embodiments described herein, the collagen used herein may be any type of collagen, including collagen types Ito XXVIII, alone or in any combination. The collagen-based matrices prepared by the methods described herein can serve as compositions for the isolation, expansion, production, and transplantation of cells and vessels.

In any of the embodiments described herein, endothelial progenitor cells can be used (e.g., to form vessels) or to generate a population of stem cells (e.g., cells expressing CD34). In one embodiment, a method is described for enhancing CD34 expression on cells. The method comprises the steps of engineering a purified collagen-based matrix comprising collagen fibrils, and contacting the matrix with endothelial progenitor cells, wherein the cells exhibit enhanced CD34 expression.

Any cell marker can be used to select and purify the cell type desired. For example, suitable markers for the selection and purification of endothelial progenitor cells include, but are not limited to, CD34, CD133, CD31, VE-Cadherin, VEGFR2, c-kit, CD45, and Tie-2. Additionally, several markers are expressed by both early angioblasts and hematopoietic elements including CD31 (PECAM- platelet endothelial cell adhesion molecule), CD34 (a general stem and progenitor cell marker), and vascular endothelial growth factor receptor type 2 (VEGFR-2 also called KDR/Flk-1). Cell markers may be used alone or in combination to select and purify the desired cell type for use in the compositions and methods herein described.

In any of the embodiments described herein, EPCs with a high proliferation capacity, otherwise known as ECFCs, are suspended in a liquid-phase, injectable collagen formulation designed to polymerize in situ to form a 3-dimensional matrix. The delivery system comprises soluble collagen, for example, soluble type I collagen, and defined polymerization reaction conditions yield natural polymeric matrices with controlled molecular composition, fibril microstructure, and mechanical properties (e.g., stiffness), for example. Varying both matrix stiffness and fibril density of the matrix predictably modulates ECFC vessel formation in vivo. Vascular networks formed by EPCs in vivo and in vitro as described can be modulated by precision-tuning specific fibril microstructure and viscoelastic parameters of the matrices, for example, the fibril density, pore size (fibril-fibril branching), shear storage modulus (G′ or elastic (solid-like) behavior), and phase angle delta (δ or the measure of the fluid (viscous)- to solid (elastic) -like behavior; δ equals 0° for Hookean solid and 90° for Newtonian fluid).

Applicants have developed type I collagen formulations derived from various collagen sources, e.g., pig skin. These formulations comprise both type I collagen monomers (single triple helical molecules) and oligomers (at least two monomers covalently crosslinked together). The presence of oligomers enhances the self-assembly potential by increasing the assembly rate and by yielding 3-dimensional matrices with distinct fibril microstructures and increased mechanical integrity (e.g., stiffness). These engineered purified collagen-based matrix formulations, together with defined polymerization conditions, are controlled to reproducibly yield 3-dimensional matrices with a range of tunable assembly kinetics (e.g. polymerization half-time), molecular compositions, and fibril microstructure-mechanical properties.

In any of the embodiments described herein, the engineered purified collagen-based matrix has a predetermined percentage of collagen oligomers based on total isolated collagen added to make the engineered matrix. In various embodiments, the predetermined percentage of collagen oligomers can be about 0.5% to about 100%, about 1% to about 100%, about 2% about 100%, about 3% to about 100%, about 5% to about 100%, about 10% to about 100%, about 15% to about 100%, about 20% to about 100%, about 30% to about 100%, about 40% to about 100%, about 50% to about 100%, about 60% to about 100%, about 70% to about 100%, about 80% to about 100%, about 90% to about 100%, about 95% to about 100%, or about 100%. In yet another embodiment, the collagen oligomers are obtained from a collagen-containing source material enriched with collagen oligomers (e.g., pig skin).

In any of the embodiments described herein, the engineered purified collagen-based matrix has an oligomer content quantified by average polymer molecular weight (AMW). As described herein, modulation of AMW can affect polymerization kinetics, fibril microstructure, molecular properties, and fibril architecture of the matrices, for example, polymerization rate, lag rate, interfibril branching, pore size, mechanical integrity (e.g., matrix stiffness), and vascularization of matrices (e.g., vacuole formation and vessel network formation within matrices). In another embodiment, the oligomer content of the purified collagen, as quantified by average polymer molecular weight, positively correlates with polymerization rate, interfibril branching, matrix stiffness, vacuole formation, and vessel network formation in vivo and in vitro.

In any of the embodiments described herein, monomer-rich collagen matrices can have an AMW of about 100 to about 280 kDa, about 250 to about 280 kDa, or about 250 to about 300 kDa, e.g., about 282 kDa. In another illustrative embodiment, oligomer-rich collagen matrices have an AMW of greater than about 300 kDa, for example, the AMW of an oligomer-rich collagen matrix can be about 300 kDa to about 2.8 MDa, about 350 kDa to about 2.8 MDa, about 400 kDa to about 2.8 MDa, about 400 kDa to about 750 kDa, about 400 kDa to about 650 kDa, about 400 kDa to about 850 kDa, about 350 kDa to about 450 kDa, about 350 kDa to about 550 kDa, about 350 kDa to about 650 kDa, about 350 kDa to about 750 kDa, about 350 kDa to about 850 kDa, about 350 kDa to about 950 kDa, about 350 kDa to about 1.5 MDa, about 350 kDa to about 2.0 MDa. In one embodiment, the oligomer-rich collagen matrices have an AMW of greater than about 2.8 MDa.

Modulation of specific biophysical parameters of a engineered purified collagen-based matrix as described, specifically fibril microstructure (length, diameter, and pore-size (fibril-fibril branching)) and mechanical properties (e.g., stiffness), regulates the fundamental behavior of resident cells. For example, multi-potential human mesenchymal stem cells entrapped within a 3D engineered purified collagen-based matrix characterized by a relatively high fibril density and stiffness (G′) show enhanced osteogenesis (bone formation), while those in a low fibril density and stiffness matrix show enhanced adipogenesis (fat formation). ECFCs grown within engineered purified collagen-based matrices in vitro show impressive vascular networks whose properties can be modulated by varying specific fibril microstructure-mechanical design parameters of the matrix as herein described.

Vacuoles can also be formed in the matrices described herein. In any of the embodiments described herein, methods of regulating vacuole density within an engineered purified collagen-based matrix are described. The method comprises the steps of engineering a purified collagen-based matrix comprising collagen fibril, and seeding the matrix with endothelial progenitor cells, wherein said seeding results in vacuole formation, and wherein the vacuole density is about 30 vacuoles/mm² to about 80 vacuoles/mm². In any of the embodiments described herein, methods of regulating vacuole density within an engineered purified collagen-based matrix are described. The method comprises the steps of engineering a purified collagen-based matrix comprising collagen fibril, and seeding the matrix with endothelial progenitor cells, wherein said seeding results in vacuole formation, and wherein the total vacuole area is about 1800 μm² to about 5000 μm².

In all of the above-described embodiments, the vacuole density can be about 10 mm² to about 200 mm², about 30 mm² to about 100 mm², about 30 mm² to about 80 mm², or about 40 mm² to about 80 mm². In all of the above-described embodiments, the total vacuole area can be about 2000 μm² to about 5000 μm², about 2500 μm² to about 5000 μm², about 3000 μm² to about 5000 μm², or about 4000 μm² to about 5000 μm².

The following examples illustrate specific embodiments in further detail. These examples are provided for illustrative purposes only and should not be construed as limiting the invention or the inventive concept in any way.

### EXAMPLE 1

### Variation of Microstructure-Mechanical Properties of Component Collagen Fibrils within a 3D Collagen ECM Modulated Mesenchymal Cell Shape and Cytoskeletal Organization

Results showed that variation of microstructure-mechanical properties of component collagen fibrils within a 3D collagen ECM modulated mesenchymal cell (MSC) shape and cytoskeletal organization. In addition, such alteration was sufficient to direct distinct growth and lineage-specific differentiation patterns of resident MSCs. Such signaling via the local 3D collagen fibril microstructure and mechanical properties occurred for MSCs cultured in “regular” medium and did not require a specialized cocktail of soluble factors. Specifically, MSCs seeded within ECMs with a fibril density of 20% and storage modulus of 44.64±8.03 Pa readily proliferated and developed a mixed cell population including adipocytes and presumably undifferentiated, spindle-shaped cells. In contrast, MSCs seeded within ECMs with a fibril density of 55% and a storage modulus of 694.05±53.09 Pa proliferated less and developed a different combination of cell types including minimal to no adipocytes, a decreased number of spindle-shaped cells, and focal aggregates of osteoblasts.

Real time RT-PCR data for LPL and CBFA1 corroborated morphology and histochemical staining results. Incubation of the constructs in the presence of “adipogenic” medium exaggerated these ECM-dependent results. There was a 9-fold increase in the number of adipocytes observed within constructs after 14 days of culture within low fibril density/stiffness ECMs in the presence of “adipogenic” medium. In contrast, MSCs cultured in high fibril density/stiffness ECMs in the presence of “adipogenic” medium showed only a moderate increase in adipogenic differentiation (approximately 2 times) but an 8-fold increase in the number of calcified bone nodules.

### EXAMPLE 2

### Differentiation Potential

Follow-up studies were conducted to determine if the initial seeding density affected the proliferative and lineage specific differentiation potential of MSCs within 3D engineered ECMs. MSCs were seeded in high fibril density/stiffness ECMs at densities ranging from 0.5×10⁴ cells/ml to 50×10⁴ cells/ml and the constructs again maintained in either “regular” or “adipogenic” media. In general, decreasing the cell seeding density caused a decrease in cell-cell interactions, an increase in cell-ECM interactions, a decrease in adipogenesis, and an increase in osteogenesis, despite the culture medium. When seeded at a low cell density, MSCs grew as focal regions, which expressed osteogenic phenotype and function, with little to no evidence of other cell types. As the initial seeding density was increased, a cell population of mixed phenotypes developed. At the highest cell density, adipocytes and undifferentiated MSCs were prominent with no evidence of osteogenesis.

The methods and compositions described herein assist in the definition of design criteria for the development of “instructive”, self-assembled, collagen-based 3D ECMs that can predictably control cell behavior and contribute to the development of functional tissues and organs for clinical applications.

### EXAMPLE 3

### Expression of Cell Surface Markers

CBFs were brought out of freezing and briefly cultured on plastic. At t=0, cells were harvested and a subset of the cells were 1) seeded within 3D ECMs; 2) seeded on plastic; or 3) subjected to flow cytometry analysis to establish t=0 results; cells were analyzed for expression of cell surface markers CD34, CD133, and PECAM; control samples representing “Cells only” and “2ndary antibody control (PECAM only)” were also analyzed. On day 6 (t=6 days), cells seeded within 3D ECM and seeded on plastic were harvested and analyzed by flow cytometry (same cell surface markers and controls were included as part of this analysis). A summary of results is provided in Table 1. CD34 expression increased for cells cultured on ECMs.

TABLE 2
Summary of the relative expression of cell surface markers CD34,
CD133, and PECAM in CBFs seeded within 3D extracellular
matrices (ECMs) compared to seeding on plastic.

| | Plastic-PS | ECM | Plastic-PS | Plastic-PureCol |
|---|---|---|---|---|
| | (t = 0) | (t = 6 days) | (t = 6 days) | (t = 6 days) |
| CD 34 | 1.3 | 20.3 | 0.5 | 0.1 |
| PECAM | 94.4 | 96.7 | 95.6 | 97.7 |
| CD 133 | 16.6* | 3.5 | 0.4 | 0.2 |
| 2° Ab | 0.2 | 2.4 | 1.5 | 1.1 |
| Control | | | | |
| (PECAM) | | | | |

Note:
Results based upon preliminary gate setting; gates set such that results obtained for cells only control were <1.3%

### EXAMPLE 4

### Flow Cytometric Analysis

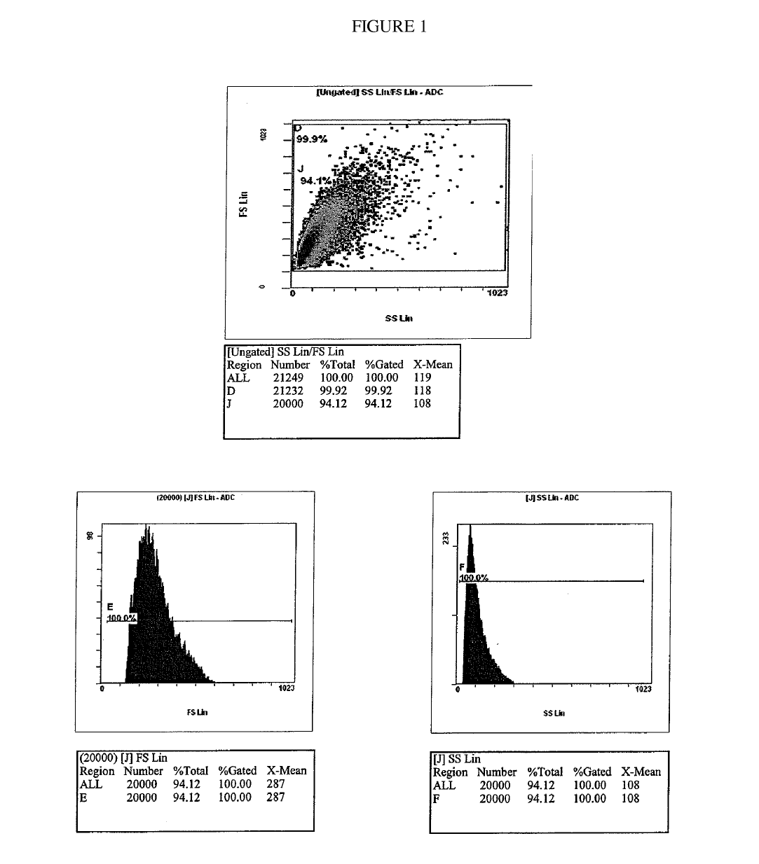

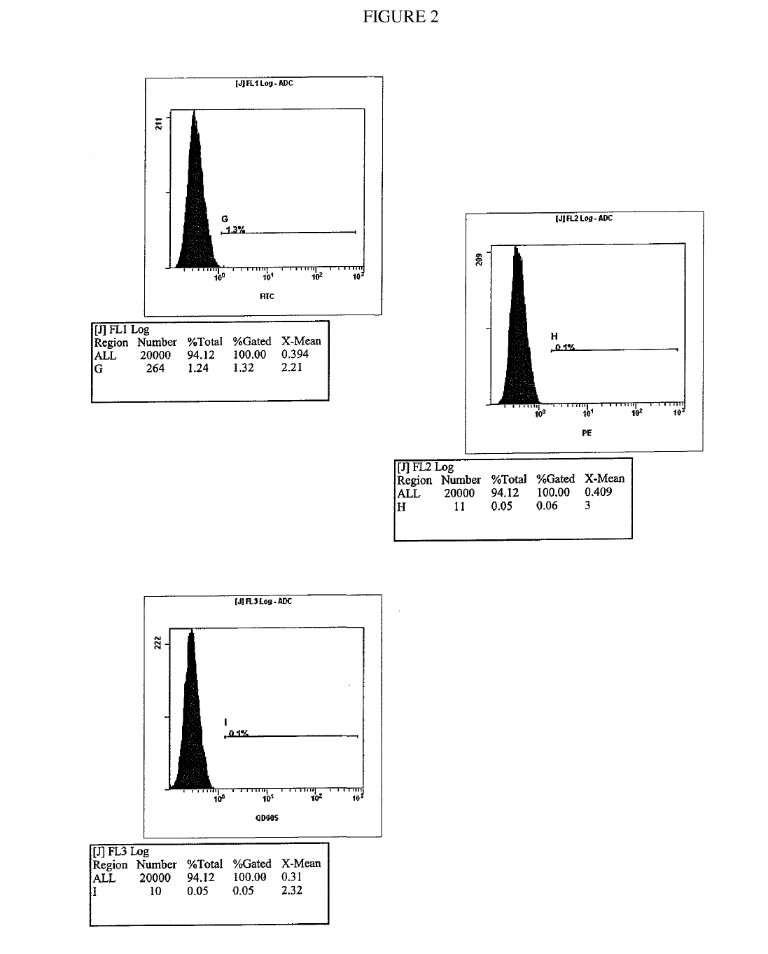

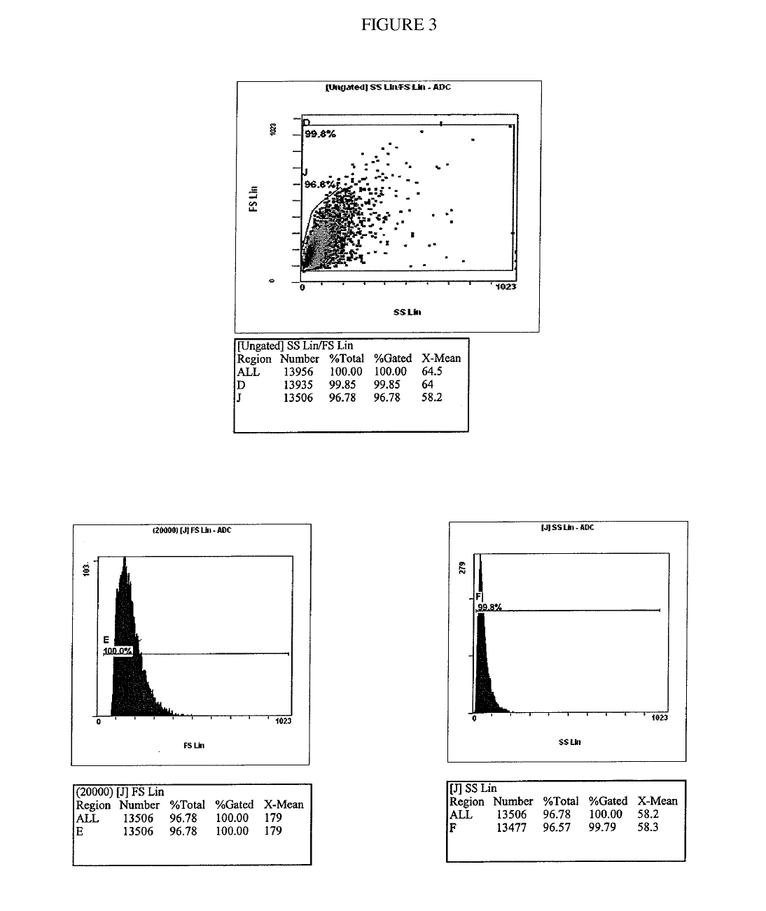

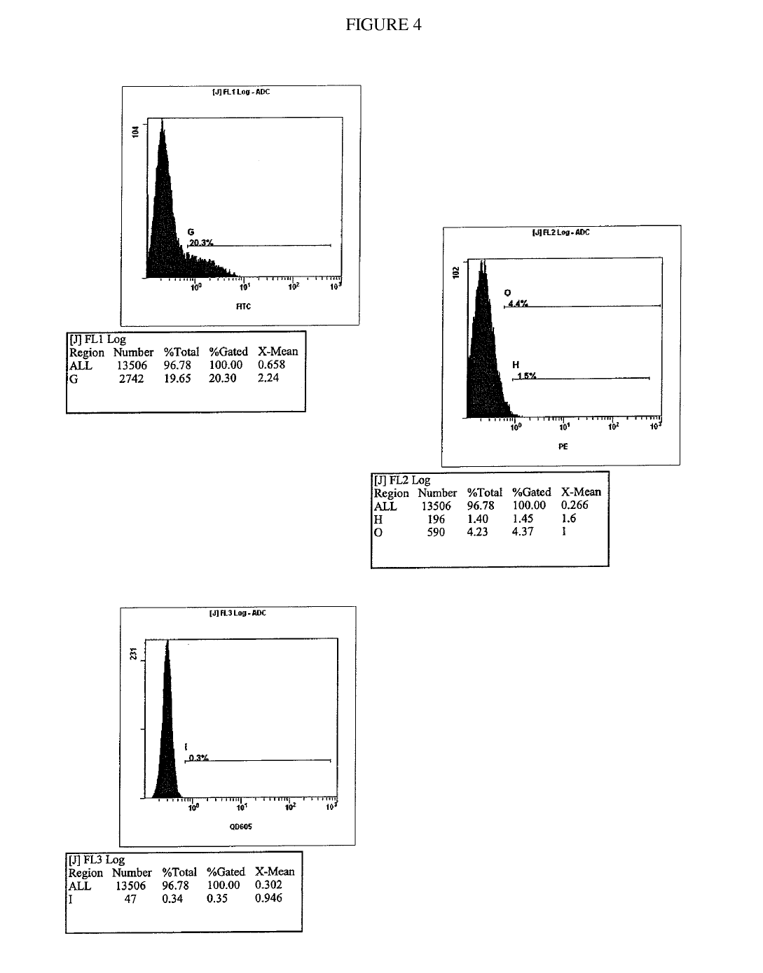

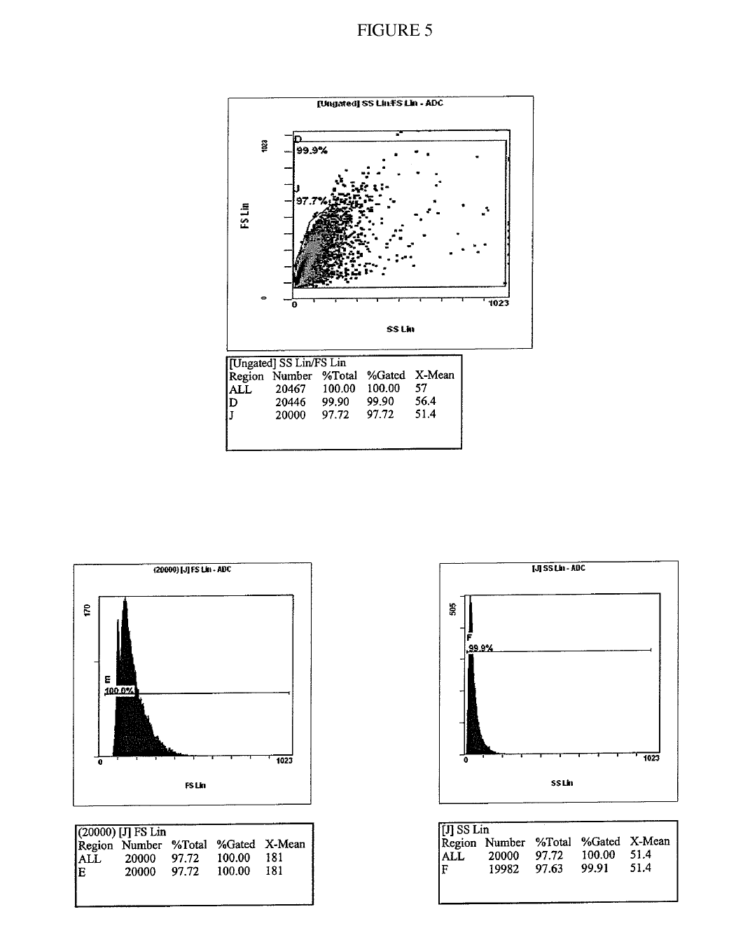

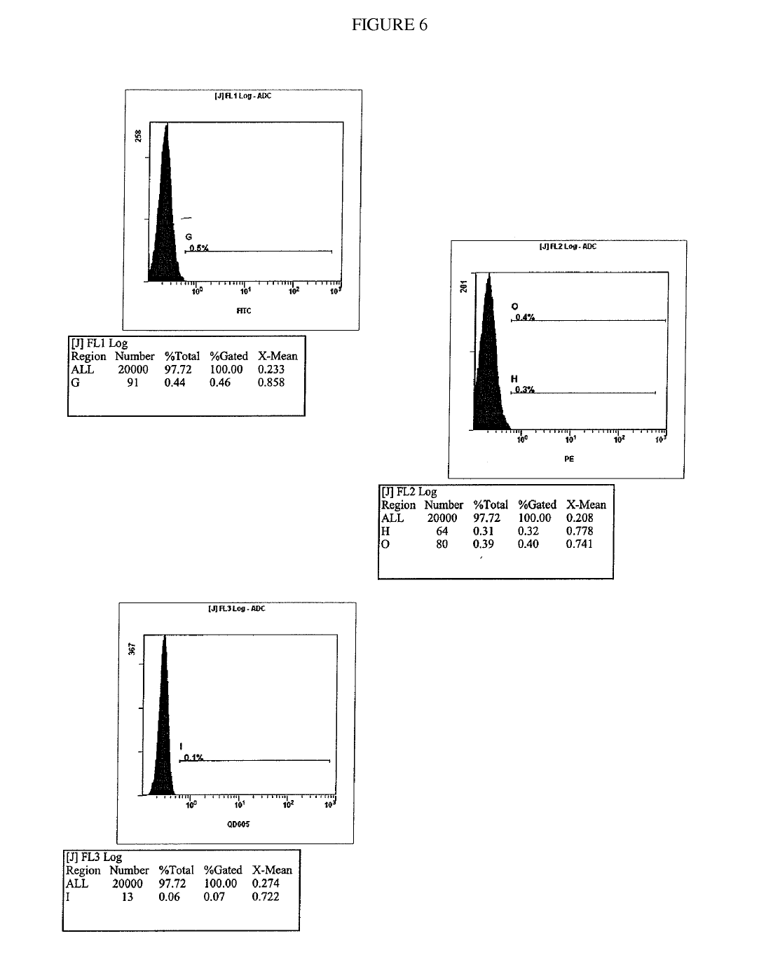

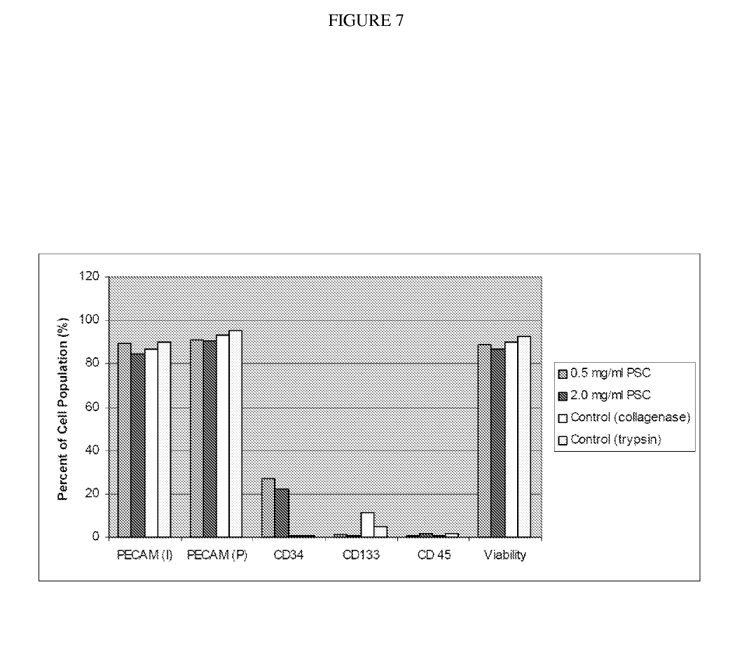

Endothelial progenitor cells (EPCs; passage 9) were seeded at cell densities of 1×10⁵ cells/ml within 3D ECMs polymerized at 0.5 mg/ml (fibril density of 6% and storage modulus of 44.64 ±8.03 Pa) and 2.0 mg/ml (fibril density of 16% and a storage modulus of 694.05±53.09 Pa) pig skin type I collagen. After 6 days of culture, cells were harvested from the ECMs using a collagenase cocktail (see Example 7). The cells then were immunofluorescently labeled for PECAM, CD34, CD 133, and CD45 and analyzed using flow cytometry (see FIGS. 1-7). The initial cell population, which was propagated on plastic, was harvested using either the collagenase or standard trypsin method and served as controls. Recovery of cells from the 0.5 mg/ml and 2.0 mg/ml ECMs was calculated at 26.5% and 21.2%, respectively. The cells grown on ECMs showed increased CD34 expression.

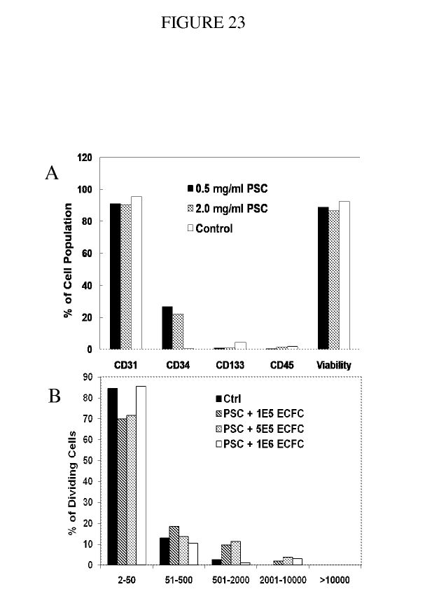

Flow cytometric analysis of the total cell population following extraction from the 3D matrix shows an intriguing shift in cell surface marker expression compared to the initial ECFC population (FIG. 23, Panel A). Specifically, the number of cells expressing CD34 increases while the number of cells expressing CD133 decreases compared to the initial population. Furthermore, expression of CD31 remains high while there is no evidence of expression of CD45, a marker specific for hematopoietic cells. In addition, the cells harvested from the matrix show a distinct shift in their proliferative potential (FIG. 23, Panel B). The differences show an increase in the number of mature endothelial cells showing low proliferative potential and an emerging small subpopulation showing enhanced proliferative potential compared to the initial ECFC population.

### EXAMPLE 5

### Endothelial Progenitor Cells (EPCS)

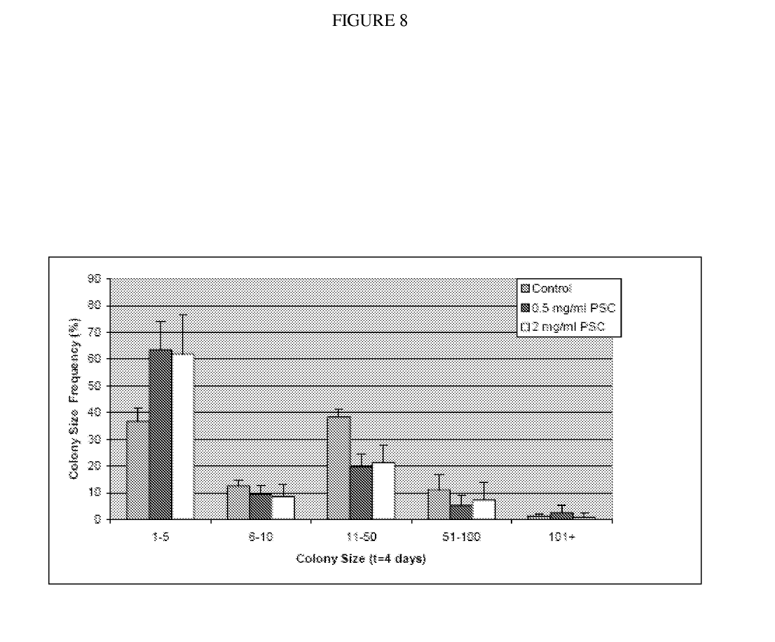

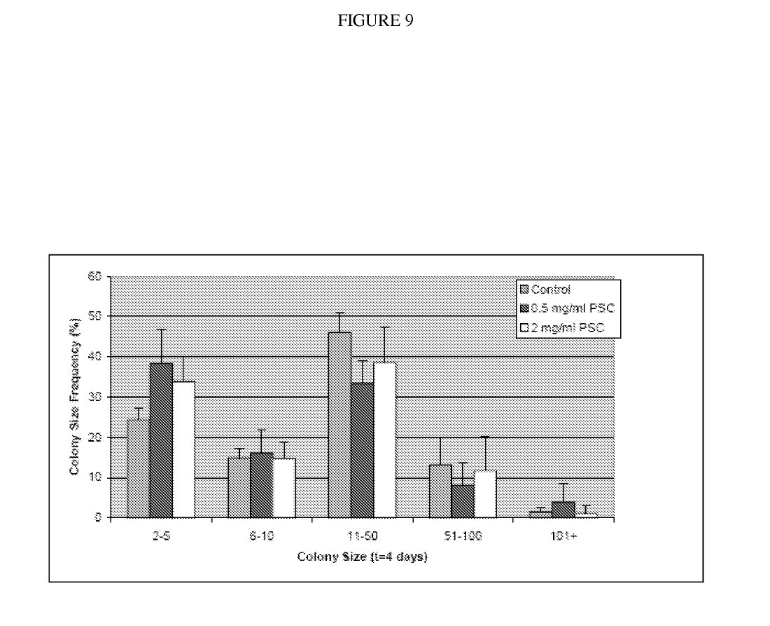

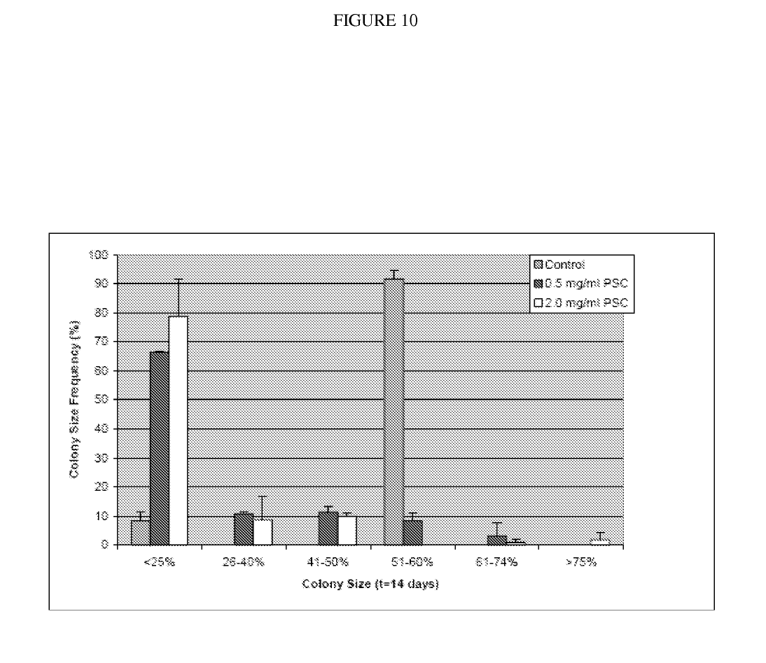

Endothelial progenitor cells (EPCs; passage 9) were seeded at cell densities of 1×10⁵ cells/ml within 3D ECMs polymerized at 0.5 mg/ml (fibril density of 6% and storage modulus of 44.64±8.03 Pa) and 2.0 mg/ml (fibril density of 16% and a storage modulus of 694.05±53.09 Pa) pig skin type I collagen (PSC). After 6 days of culture, cells were harvested from the ECMs using a collagenase cocktail (see Example 7). The cells then were analyzed using a colony forming assay. The colony forming potential for the EPCs prior to seeding within the matrices also was determined and served as a Control. The percentage of dividing cells was at 99.1±0.5%, 95.3±4.8%, and 96.3±3.9% for Control, 0.5 mg/ml PSC, and 2 mg/ml PSC groups, respectively. The colony size formed by an EPC population before being seeded within 3D ECMs (Ctrl) and after being seeded at cell densities of 1×10⁵ cells/ml within 3D ECMs polymerized 0.5 mg/ml and 2.0 mg/ml was measured at 4 days (FIGS. 8) and 14 days, (FIG. 10). Note the shift in the colony forming potential for the cells seeded under the different conditions. These data include single cell events. Measurements of colonies containing at least 2 cells at 4 days are shown in FIG. 9.

EXAMPLE 6 ENDOTHELIAL PROGENITOR CELLS (EPCS)

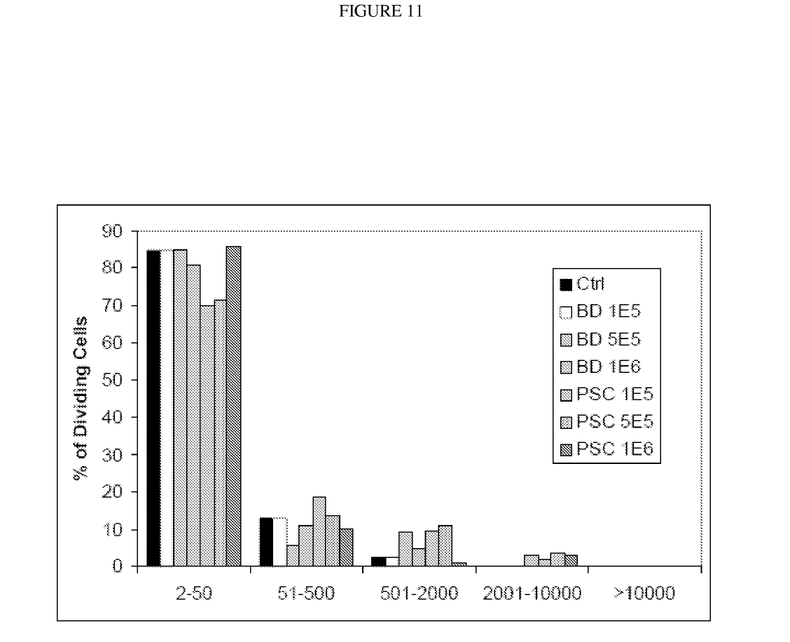

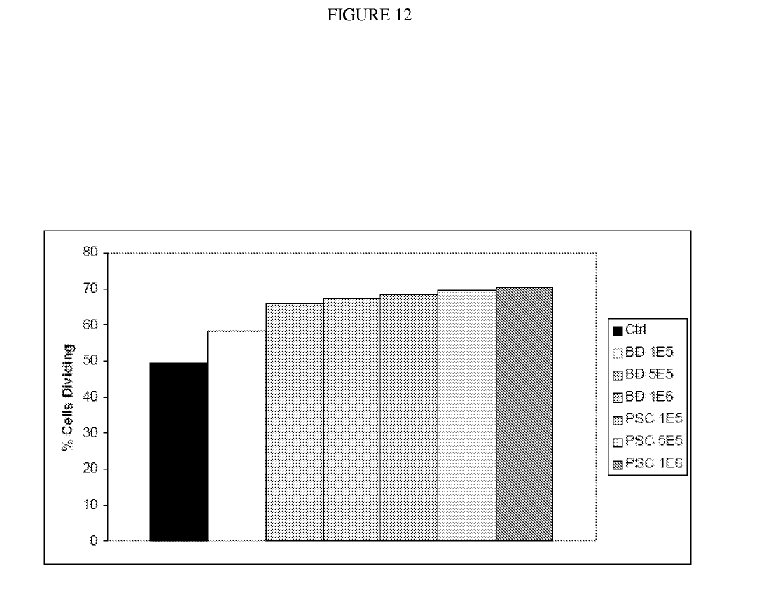

Endothelial progenitor cells (EPCs) were seeded at cell densities of 1×10⁵, 5×10⁵, and 1×10⁶ cells/ml within 3D ECMs prepared with either pig skin type I collagen (1.5 mg/ml) or type I collagen (1.5 mg/ml; BD Biosciences)+fibronectin (1 μg/ml) and maintained for 7 days. PSC and BD were used as designators for the pig skin collagen and commercial collagen+fibronectin formulations, respectively. After 7 days of culture, cells were harvested from the ECMs using an enzyme cocktail (see Example 7). The cells were then analyzed using a colony forming assay. The colony forming potential for the EPCs prior to seeding within the matrices also was determined and served as a Control (Ctrl). A shift in the colony forming potential was found for the cells seeded under different conditions. EPCs grown within PSC showed increased colony forming potential even at low seeding densities (FIG. 11). An increase in the percentage of dividing cells was obtained after EPCs were seeded within 3D ECMs (FIG. 12). Upon comparison of EPCs grown within BD and PSC ECM formulations, it was observed that EPCs seeded at a given cell density showed the greatest proliferative potential within the PSC formulation.

### EXAMPLE 7

### Protocol for Removing Cells from Constructs with Collagenase

This protocol was developed and optimized for the effective recovery of single cells from 3D ECM constructs while maintaining maximum viability. The collagenase is from Worthington, Type IV, and is used at a 500 U/ml concentration in the EPC extraction media. The dispase (Neutral protease) is from Worthington, and is used in a range from 1-2.4 U/ml, preferably 2.4 U/ml, in the extraction media with the collagenase. The Extraction Media is the EPC media from Lonza (EGM-2, CC3162, including the singlequots and extra Hyclone serum which makes it 12% serum) with additional serum from Hyclone to make it 50% serum. Additional ingredients include Gibco TripLE trypsin, the regular EPC media with 12% serum, and Trypan Blue. Large orifice tips and pipettes are to be used when pipetting the cells. The following steps are then performed:

1. Make the Extraction Media (50% serum media), warm to 37° C. Calculate the amount of collagenase/dispase that will be needed (usually 1 ml per construct from a 24 well plate plus extra for loss during filtering). Weigh the correct amount of collagenase and dispase into a single tube and add the correct amount of Extraction Media. Sterile filter with a 0.2 μm syringe filter. Use immediately.

2. Into a 15 ml tube add 5 ml of the sterile collagenase/dispase solution.

3. With sterile forceps place 5 constructs from a 24 well plate into the tube.

4. Shake at 120 rpm, 37° C. for 20 minutes. Keep the tube at a 45° angle to increase the surface area. Flick the tube frequently.

5. Add an equal volume of Extraction Media. Pipet up and down gently.

6. Centrifuge at 1000 rpm for 5 minutes at room temperature.

7. Remove the supernatant and rack the tube with the remaining pellet.

8. Add 5 mL of regular EPC media, pipet up and down gently and centrifuge as in number 6.

9. Remove the supernatant and rack the tube with the remaining pellet.

10. Add 100 μl Gibco TrypLE and pipet up and down gently.

11. Shake at 120 rpm, 37° C. for 15 minutes. Flick the tube frequently.

12. Add 100 μl regular EPC media to stop the trypsin and pipet to mix.

13. Take 15 μl of the sample and add to 15 μl Trypan blue.

14. Do a cell count.

### EXAMPLE 8

### Endothelial Colony Forming Cells (ECFCS)

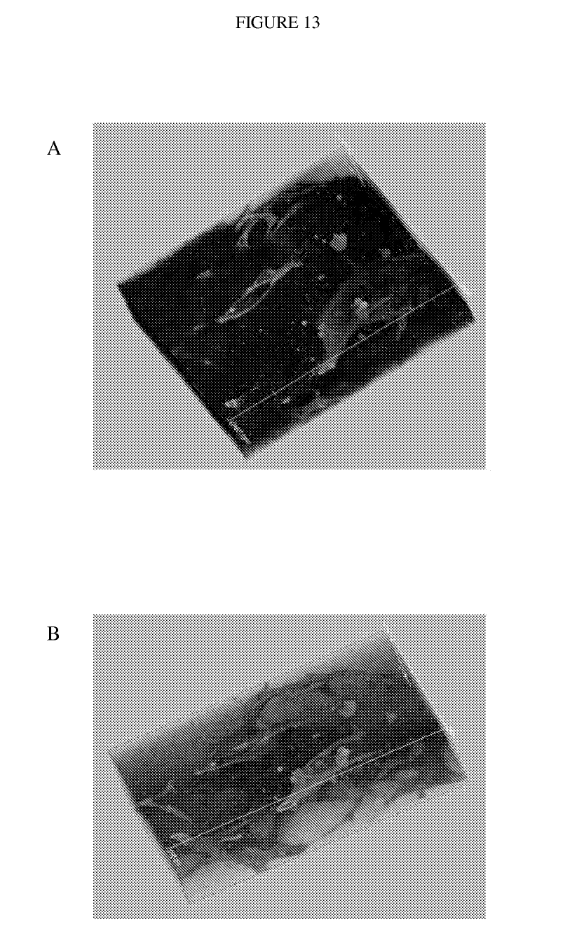

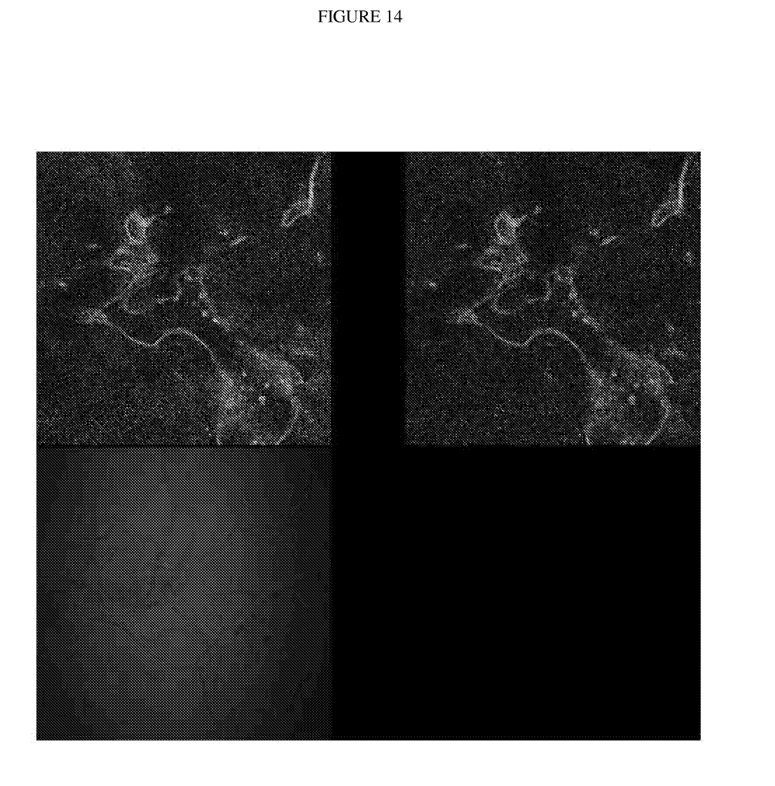

Endothelial colony forming cells (ECFCs) were seeded within engineered extracellular matrices prepared from pig skin collagen. ECFCs (bright white) were labeled with FITC conjugated UEA-1 lectin and collagen fibril microstructure was simultaneously visualized using 488 nm reflected light (FIG. 13). ECFCs formed endothelial-lined microvessels, some of which contained round, viable cells (FIG. 14).

### EXAMPLE 9

### Type I Collagen 3D ECM Microenvironment Alters ECFC Vascular Network Formation in Vitro

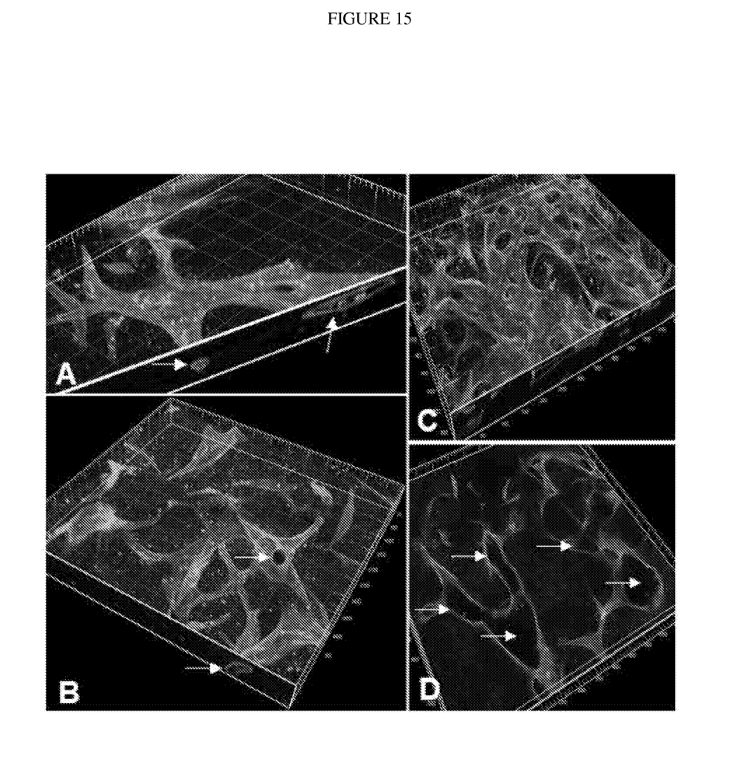

Endothelial colony forming cells (ECFCs) were isolated as previously described and suspended in collagen solutions prior to polymerization to ensure a uniform distribution throughout the type I collagen 3D ECM. To investigate the role of cell-cell interactions in ECM guidance of vascular network formation ECFCs were seeded at a density of about 1×10⁵ to about 10⁶ cells/mL within engineered extracellular matrices and cultured for 8 or 14 days (FIG. 15). Three dimensional images were taken that illustrate the differences in vascular network development by ECFCs prepared with pig skin collagen concentration, fibril volume fraction, and stiffness (G′) of 2 mg/ml, 38%, and 767 Pa (FIG. 15, panel A) compared to 0.5 mg/ml, 9%, and 48 Pa (FIG. 15, panel B) after 8 days. FIG. 15, panels C and D represent an extensive vascular network produced by ECFCs after 14 days of culture within an engineered ECM. Panel C shows the network of ECFCs and Panel D provides a volume slice clearly demonstrating the lumens present in the vascular network. Fluorescence and reflection confocal microscopy were used to visualize the ECFC derived vascular structures and the surrounding collagen ECM respectively (FIG. 15). ECFCs (bright white) were labeled with FITC conjugated UEA-1 lectin and collagen fibril microstructure was simultaneously visualized using 488 nm reflected light (arrows denote visible lumens). The major tick mark on all images equals 50μm.

These studies show a qualitative difference in structure formation and regression in the four ECM environments tested. Even in the absence of phorbol esters the ECMs were able to direct ECFC vascular structure formation. Vascular networks were largest and most complex around 72 hours and then these networks started to regress. Less vascular structure regression occurs in the pig skin collagen ECMs.

### EXAMPLE 10

### Mechanical Properties of Type I Collagen ECMS

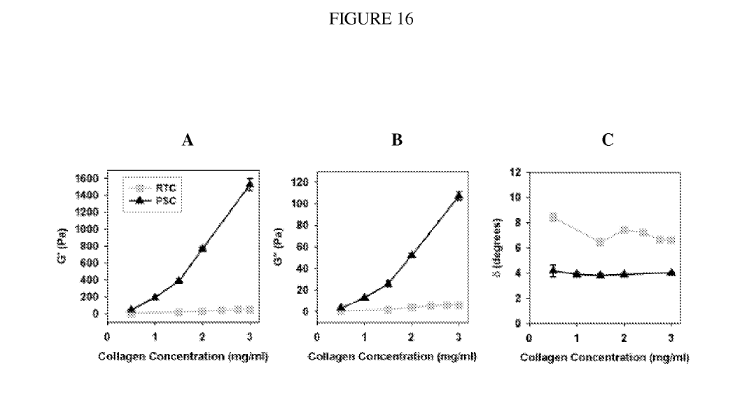

An experiment was performed to study the microstructural-mechanical properties of two sources of collagen. An ECM from pig skin collagen was compared to an ECM from commercially available rat tail collagen (Becton-Dickinson) over a range of collagen concentrations, from about 0.5 mg/ml to about 3.0 mg/ml. Engineered 3D ECMs from rat tail and pig skin collagen showed distinct relationships between fibril microstructure and mechanical properties. FIG. 16, Panel A, shows the shear storage modulus, or stiffness, over a range of collagen concentrations for pig skin compared to rat tail collagen. The pig skin collagen demonstrated a broader range for shear storage modulus than the rat tail collagen over the range of collagen concentrations measured. FIG. 16, Panel B, shows the shear storage modulus over the same range of collagen concentrations. Again, the pig skin collagen demonstrated a broader range of shear storage modulus. FIG. 16, Panel C, depicts delta, which is the phase shift of the strain and stress waves over the range of collagen concentrations. The rat tail collagen was found to have a higher delta, and thus a more viscous response.

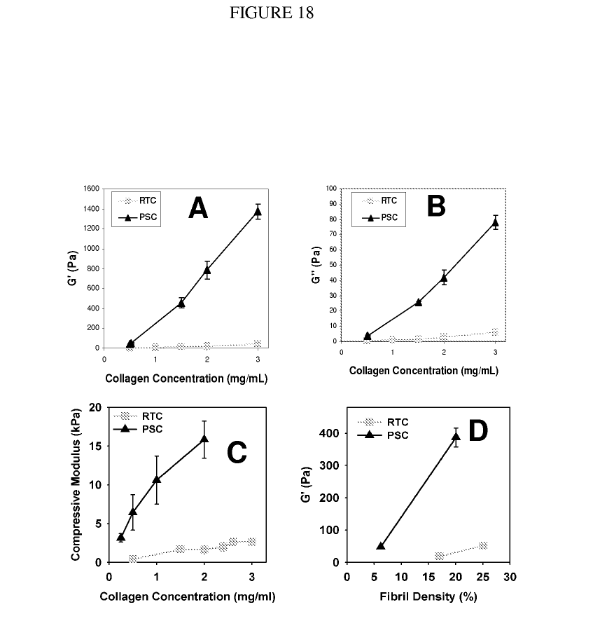

A Sirius red assay was used to verify the collagen concentration of both sources. Viscoelastic properties were determined for each collagen source using a TA Instruments AR-2000 rheometer adapted with a 40-mm plate geometry and a humidity trap. All samples were tested under oscillatory shear and at least 4 repetitions of each sample were completed. Each sample was allowed to self-assemble (polymerize) for 1 hour at 37° C. prior to strain sweep and unconfined compression analyses. A strain sweep was conducted in the linear viscoelastic range over a strain range of 1×10⁻⁴ to 1×10⁻² and storage modulus (G′) (stiffness) and the loss modulus (G″) calculated. Each sample was then tested in unconfined compression and the compressive stiffness determined (FIG. 18). Confocal reflection microscopy was used to visualize the 3D fibril microstructure and the fibril volume fraction (fibril density) was determined as previously described [Voytik-Harbin, J. Biomech. Eng., 124(2): 214-22 (2002); incorporated herein by reference] (FIG. 18). The mechanical properties of the 3D ECMs from type I pig skin collagen (PSC) and rat tail collagen (RTC) are shown in FIG. 18, Panel A, as shear storage modulus (G′) of RTC and PSC ECMs versus collagen concentration; FIG. 18, Panel B, as shear loss modulus (G″) of RTC and PSC ECMs versus collagen concentration; FIG. 18, Panel C) compressive modulus of RTC and PSC ECMs versus collagen concentration; and FIG. 18, Panel D, as shear storage modulus (G′) versus fibril density for RTC and PSC ECMs.

The relationship between stiffness (G′) and fibril density is different for pig skin and rat tail collagen ECMs (FIG. 18, Panel D). As a result the stiffness (G′) or fibril density for ECMs from the two different collagen sources can be matched but stiffness (G′) and fibril density can not be matched simultaneously. However, using two collagen sources and 4 ECM microenvironments allowed the effects of the two parameters, stiffness (G′) and fibril density, on the ability of the ECM to influence ECFC vascular structure formation to be determined.

### EXAMPLE 11

### Characterization of Engineered ECM Microstructural-Mechanical Properties

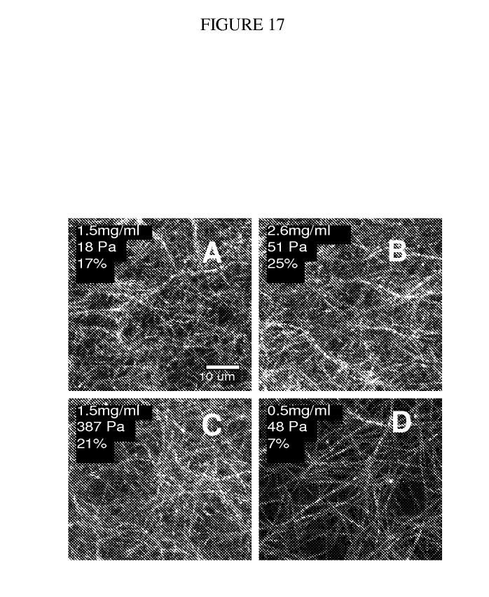

Different ECM microenvironments were tested in these experiments and rat tail and pig skin collagen were found to produce ECMs with distinct mechanical properties. Representative 2D projections of confocal reflection image stacks comparing the fibril microstructure for engineered ECMs prepared using commercial (Panels A and B) and pig skin (Panels C and D) collagen sources are shown in FIG. 17.

Self-assembly conditions of both collagen sources were adjusted to yield engineered ECMs with the same fibril volume fraction (Panels A and C) or storage modulus (G′, stiffness; Panels B and D). The rat tail collagen construct had a stiffness of 18 Pa and the pig skin collagen system had a stiffness of 387 Pa. FIG. 17 (Panels B and D) show a new set of rat tail and pig skin constructs designed to be matched in stiffness. Initial collagen concentration, G′, and fibril volume fraction data are provided. FIG. 18, Panel D, shows the relationship between shear storage modulus and fibril density. This relationship was distinct in the two collagen sources, revealing that either collagen concentration or fibril density could be matched, but not both simultaneously.

From these studies, it is shown that for a given collagen concentration, ECMs from the pig skin collagen have a greater fibril density and stiffness (G′). Further, over the range of collagen concentrations investigated pig skin collagen yielded ECMs with a broader range of fibril microstructure and mechanical properties. From these mechanical studies four ECMs, two from each collagen source, that have either the same fibril density or stiffness (G′) were selected to investigate the effects of ECM mechanical properties on ECFC vascular network formation (FIG. 17).

### EXAMPLE 12

### Vascular Network Formation

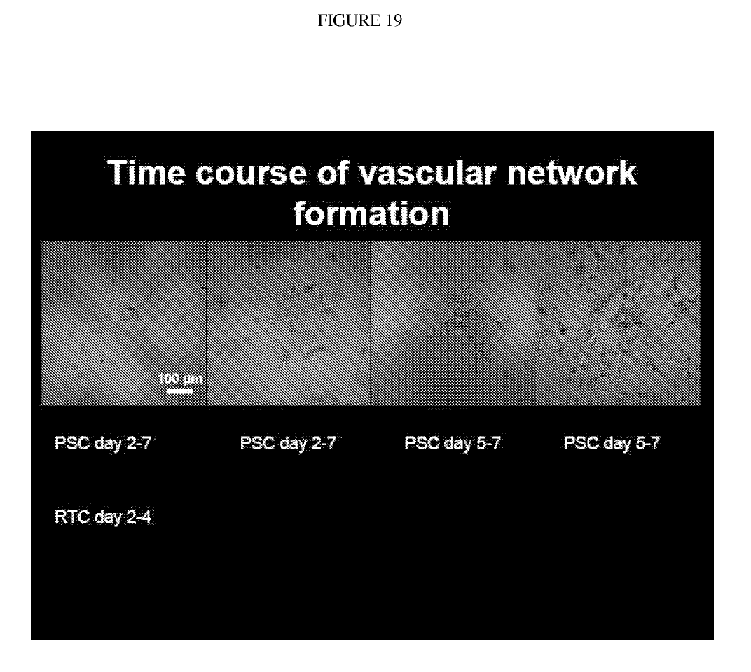

Vascular network formation in culture was examined over time (FIG. 19). The first panel shows the smallest and least complex structures which typically appear at 2 days of culture. The structures persist in the pig skin collagen system but regress in the rat tail collagen system around day 5. The next panel depicts a step up in vascular structural complexity that also appears at around day 2 in the pig collagen system. These structures are not seen in the rat tail collagen system. The third and fourth panels show the two most complex vascular structures which appear at around day 5 in culture. Again such complex structures are only seen in the pig skin collagen system.

### EXAMPLE 13

### Type I Collagen 3D ECM Microenvironment Alters ECFC Vascular Network Formation in Vitro

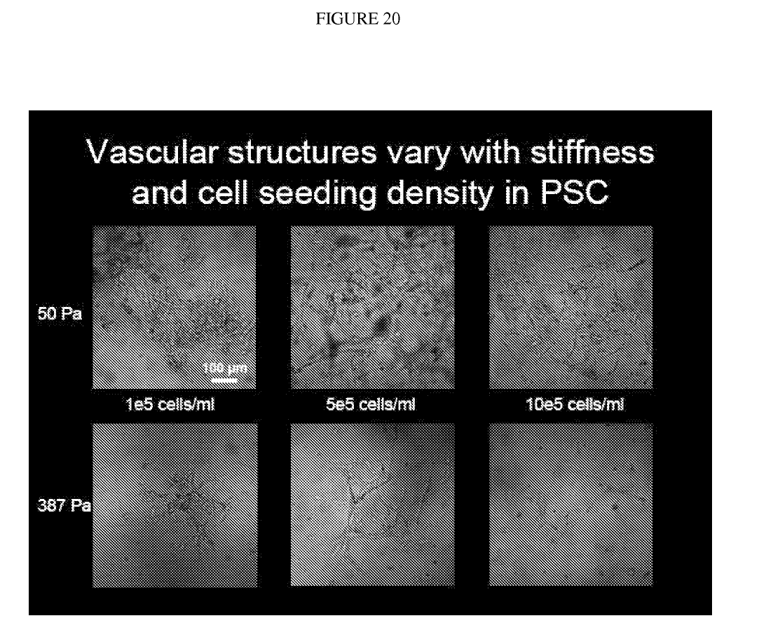

Vascular structure complexity was found to vary with stiffness and cell seeding density in the pig skin collagen system (FIG. 20). The top row depicts representative structures in the 50 Pa, or low stiffness, pig collagen constructs. The first column shows a seeding density of 1×10⁵ cells/ml. The second column depicts a seeding density of 5×10⁵ cells/ml. The structures are larger and more complex at this seeding density. The third column is representative of 1×10⁶ cells/ml, wherein the structures seen were typically smaller and less complex than at the seeding density of 5×10⁵ cells/ml.

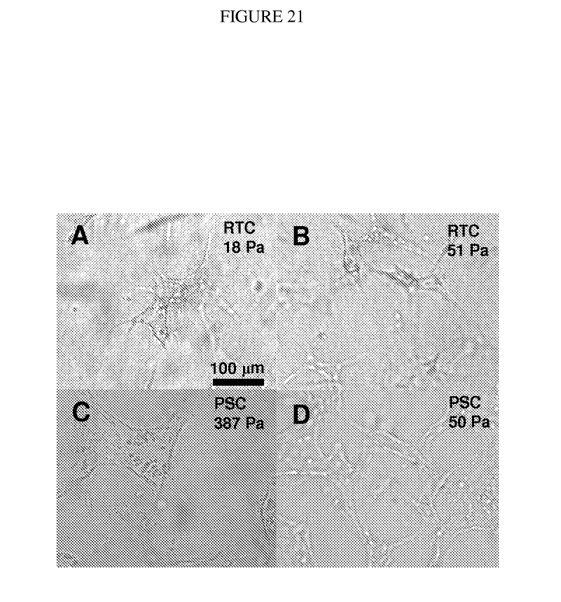

Difference in stiffness (G′) and fibril density affected size and complexity of ECFC vascular structures (FIG. 21). Vascular Structures were seen in all four ECMs during the seven day culture period. The pig skin collagen ECM with 48 Pa matrix stiffness (G′) and 7% fibril density qualitatively had the largest and most complex vascular structures. This ECM had the lowest fibril density of the four ECMs tested and had an intermediate stiffness, indicating that both parameters are important in directing ECFC behavior. Brightfield images showed that ECMs from rat tail collagen (RTC) (Panels A and B) and pig skin collagen (PSC) (Panels C and D) were able to support ECFC vascular structure formation to varying degrees. ECMs depicted in Panels A and C have the same fibril density, while ECMs depicted in Panels B and D have the same stiffness (G′), shown in Pascals (Pa).

An increase in ECFC seeding density resulted in larger and more complex vascular structures in the rat tail collagen ECMs. In the pig skin collagen ECMs vascular structures seen with both seeding densities were of similar size and complexity as those seen in the rat tail collagen ECMs at a seeding density of 10⁶ cells/mL. One interpretation of these initial results is that the microstructure of the pig skin ECMs are better able to transmit ECFC generated mechanical signals that aid in the formation of mutlicellular structures prior to the cell-cell contacts being established.

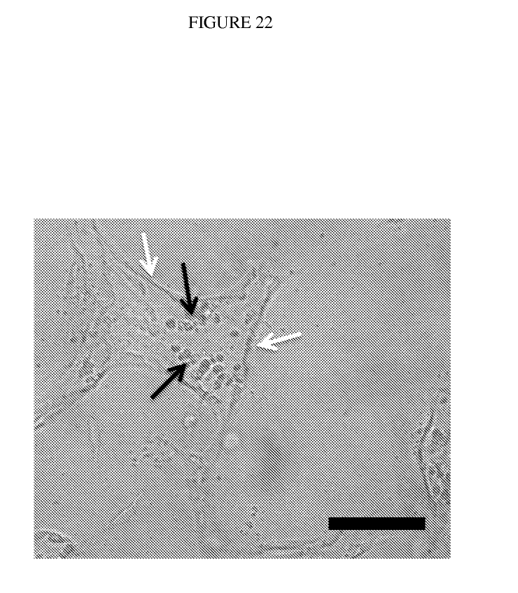

ECFCs seeded within 3D collagen matrices undergo a morphogenesis process including vacuolization, cell proliferation, and a balance between cell-cell and cell-matrix interactions to form lumen-containing vessels. Under specific conditions, distinct populations of rounded cells are identifiable within the lumens of vessels, reminiscent of blood island formation as occurs in vasculogenesis during development (FIG. 22).

### EXAMPLE 14

### Pig Skin and Rat Tail Type I Collagen ECMS Direct ECFC Blood Vessel Formation in Vivo

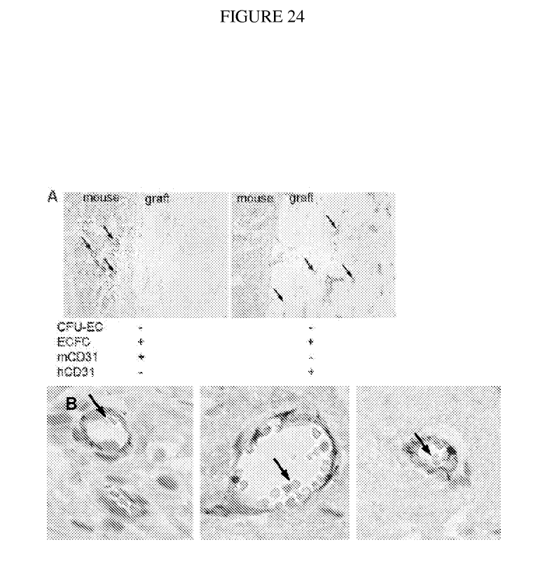

ECFCs were suspended in either pig skin or rat tail collagen solution at 2×10⁶ cells/mL and 1 mL of the solution was added to a 12 well tissue culture plate. The ECM polymerized for 20 minutes at 37° C. and then 2 mL of warm EGM-2 (Lonza, Basel, Switzerland) media was added. The ECFCs in ECMs were cultured overnight. The ECMs were bisected and then implanted subcutaneously into the flank of a mouse as previously described. NOD/SCID/γ_{c}^{null} mice (T-, B-, & NK cell deficient, impaired complement) were chosen as the animal model to alleviate xenogenic barriers associated with implantation of human cells. After 14 days the mice were euthanized and the collagen ECMs were harvested, fixed in a formalin free fixative (BD Pharmingen, San Diego, Calif.), and embedded in paraffin. Sections 6 μm thick were cut and either stained with Hematoxylin and Eosin (H&E) or with antibodies to either mouse or human CD31 as previously described. A monoclonal mouse anti-human CD31 antibody (clone JC/70A, AbCam, Cambridge, Mass.) and an anti-mouse CD 31 antibody (clone mec 13.3, BD Pharmingen, San Diego, Calif.) were used to differentiate between vessels formed from human ECFCs and host vessels that may have invaded the ECM (FIG. 24).

FIG. 24, Panel A shows a photomicrograph (original magnification, ×20) of cellularized ECMs and surrounding mouse tissue. The two panels show consecutive sections of the same ECM stained with anti-mouse CD31 (mCD31) and anti-human CD31 (hCD31) to identify either mouse or human vessels respectively. mCD31 does not cross-react with human ECFCs within the ECM and hCD31 does not cross-react with mouse ECs in vessels in the host tissue. FIG. 24, Panel B shows a photomicrograph (original magnification, ×100) of ECFC vessels stained with hCD31. ECFC vessels and capillaries in the ECM are perfused with mouse red blood cells (arrows) indicating anastomoses with mouse blood vessels.

A collagen-fibronectin ECM, previously shown to facilitate ECFC vessel formation, was used as a positive control. The mechanical properties of the collagen-fibronectin ECM were tested and the matrix stiffness (G′) and fibril density were determined. Both the stiffness and fibril density were not significantly different then the 18 Pa rat tail collagen ECM (data not shown). These studies demonstrate that human umbilical cord blood derived ECFCs form blood vessels de novo in ECMs of both pig skin and rat tail collagen with matched fibril density (data not shown). Qualitative differences in the number of human vessels formed and the size of the vessels formed in the pig skin and rat tail collagen ECMs were seen.

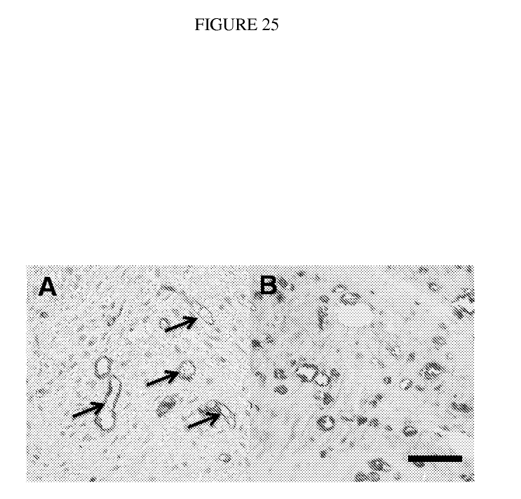

The ability of ECFCs to form vessels with anastomoses to host vessels in vivo is dependent upon the fibril microstructure-mechanical properties of the delivery collagen matrix (FIG. 25). Histological cross-sections showing matrix-dependent ECFC response 2 weeks following subcutaneous implantation within NOD/SCID mice are shown. ECFCs were implanted within collagen matrices that varied in fibril density and stiffness, (FIG. 25, Panel A) 12% and 30 Pa (0.5 mg/ml); and (FIG. 25, Panel B) 21% and 650 Pa (2.5 mg/ml). Sections were stained for anti-human CD31 and counterstained with H&E. Numerous functional vessels (arrows) were noted within the 50 Pa matrix. In contrast, vessels formed within the 650 Pa matrix failed to anastomose with host vessels.

### EXAMPLE 15

### USSCS Aid in ECFC Blood Vessel Formation In a Type I Collagen 3D ECM in Vivo

ECFCs and USSCs were suspended in fibronectin-rat tail collagen solution at a ratio of 4:1 while maintaining the total cell seeding density at 2×10⁶ cells/mL. ECFCs and USSCs were also suspended individually in fibronectin-rat tail collagen solution at 2×10⁶ cells/mL. As before, 1 mL of the solution was added to a 12 well tissue culture plate. The ECM was allowed to polymerize for 20 minutes at 37° C. and then 2 mL of warm EGM-2 media was added. The ECFCs in ECMs were cultured overnight. The ECMs were bisected and then implanted subcutaneously into the flank of a NOD/SCID/γ_{c}^{null} mouse as previously described.

After 14 days, the mice were euthanized and the collagen ECMs were harvested, fixed in a formalin free fixative (BD Pharmingen, San Diego, Calif.), embedded in paraffin and 6 μm sections were cut. Sections were either stained with Hematoxylin and Eosin (H&E) or with antibodies to either mouse or human CD31 as previously described.

ECFC and USSC co-culture in rat tail collagen-fibronectin ECMs formed 26.14±8.32 (mean±standard deviation) functional blood vessels, while ECFCs embedded alone formed 16.83±7.12 functional blood vessel showing USSC stabilization of ECFC derived vessels. USSC seeded alone in a rat tail type I collagen ECM significantly contracted the ECM but did not form any blood vessels (data not shown).

USSCs commit to different lineages within the ECM. ECMs implanted with both ECFCs and USSCs stain positive with Von Kossa, an indication of calcium deposition, and 1% Alcian blue in dye in glacial acetic acid, indicating chondrogenic differentiation (data not shown).

### EXAMPLE 16

### Localized Delivery of ECFC in a Type I Collagen 3D ECM Impacts Wound Healing in Vivo

Type I collagen 3D ECMs direct ECFC vessel formation and improve wound healing. A full thickness skin wound model was developed which utilized NOD/SCID/γ_{c}^{null} mice. A 5 mm circular punch biopsy wand was used to remove a 5 mm area of full thickness skin. ECFCs were injected into the periphery of the wound in either EBM-2, a basal media, or in the collagen-fibronectin ECM. Each wound received four injections of 25 μL evenly spaced around the periphery using a 100 μL Hamilton syringe. EBM-2 or collagen-fibronectin without ECFCs was injected into the periphery of the wound as a negative control. Pictures of the wounds were taken daily for two weeks and the wound areas were calculated using Metamorph (Molecular Devices, Sunnyvale, Calif.). The change in wound area from initial wounding to the end of the study was calculated and then normalized by initial wound size. Results show localized delivery of ECFCs in rat tail collagen-fibronectin ECM reduce the wounds to 6.2±3.1% (mean±standard deviation) of original wound size compared to EBM-2 alone 19.6±17.5%, ECM alone 19.3±16.2%, and ECFCs in ECM 21.6±25% of original wound size (n=3) (data not shown).

### EXAMPLE 17

### Culture of ECFCS

Human umbilical cord blood ECFCs were obtained from Endgenitor Technologies, Inc (Indianapolis, Ind.) and cultured as previously described (Ingram et al., 2004, Blood, 104(9): 2752-60). ECFCs were typically used between about passage 6 and 8.

### EXAMPLE 18

### Polymerization of 3D Collagen Matrix

Cellularized collagen matrix implants were cast as previously described (Schechner, 2000). Generally, cultured ECFCs (2×10⁶ cells/ml) were suspended in a solution of 0.5 to 3.5 mg/ml (final concentration) rat tail type I collagen (BD Biosciences, Bedford, Mass.), 100 ng/ml human fibronectin (Millipore, Temecula, Calif.), 1.5 mg/ml sodium bicarbonate (Sigma, St. Louis, Mo.), 10% FBS, 25 mM HEPES, 30% complete EGM-2, and EBM-2 (Lanza, Walkersville, Md.). Collagen-cell suspensions were kept at 4° C. during mixing and pH adjusted to 7.4 using 1 M NaOH. Volumes (1 ml) were pipetted into wells of 12-well plates, allowed to polymerize at 37° C. for 30 min, and covered with complete EGM-2 for overnight incubation at 37° C., 5% CO₂.

### EXAMPLE 19

### Microstructural Analysis of 3D Collagen Matrices

Confocal reflection microscopy (CRM) was used to visualize the 3D fibril microstructure of collagen matrices (Voytik-Harbin, 2001). Matrices were polymerized in Lab-Tek™ chambered coverglass slides (Nunc, Thermo Fisher Scientific, Rochester, N.Y.), overlaid with PBS, and imaged on an Olympus Fluoview™ FV1000 confocal system adapted to an Olympus 1X81 inverted microscope with a 60× UPlanSApo water immersion objective (Olympus, Tokyo, Japan). Images were collected at random locations within at least 2 independent matrices (n=10 images per matrix formulation). Fibril volume fraction (fibril density) was calculated as the percentage of fibril voxels (size 01×0.1×0.1 μm³) to total image voxels for each image using Matlab (Mathworks, Natick, Mass.). Fibril voxels were determined by applying a threshold value chosen mathematically as the center of the concave bend in the sigmoidal decay curve of fibril volume fraction versus threshold value for that image. From original CRM images, fibril diameters of 16 fibrils were measured in 3 images for each matrix formulation (n-48 fibrils) using Imaris software (Bitplane Inc, St. Paul, Minn.).

Characterization of Matrix Physical Properties:

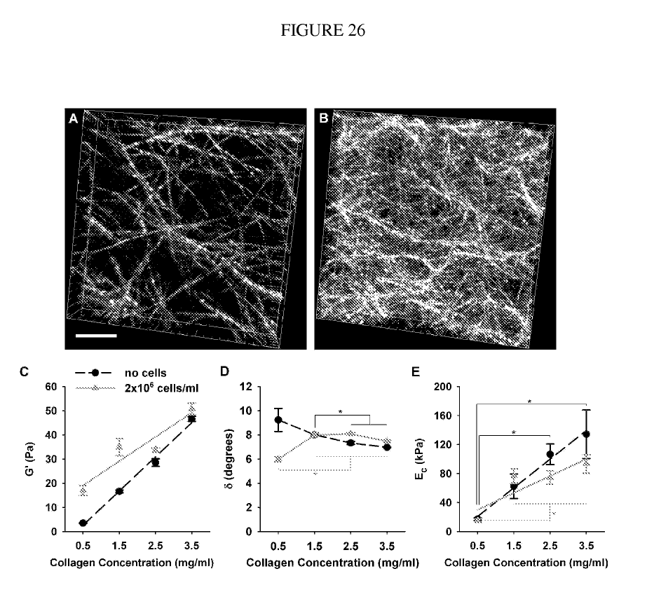

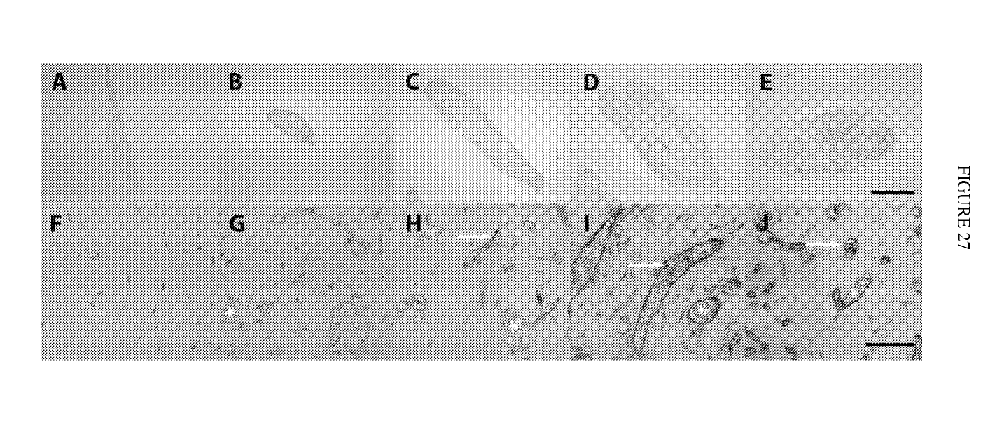

Collagen concentration was varied to systematically vary matrix physical properties (e.g. fibril density and stiffness) known to affect in vitro EPC vessel formation. CRM analysis showed that varied collagen concentration significantly altered local 3D fibril microstructure (FIG. 26, Panels A and B). Fibril volume fraction (fibril density) calculated from CRM images increased linearly with increasing collagen concentration. Average fibril diameter did not change with collagen concentration (396±53 nm, mean±standard deviation of all concentrations). Mechanical analysis showed that matrix G′ (indicates stiffness) also significantly increased linearly with increasing concentration (FIG. 26, Panel C, p<0.05 at each concentration interval). Small δ indicated that matrix shear response was dominated by the collagen fibril/solid phase of the matrices (FIG. 26, Panel D). δ showed a small, but significant dependence on collagen concentration. δ is not usually dependent on collagen concentration, suggesting that the added matrix components (fibronectin or FBS) may have somehow contributed to this non-linear behavior.

Similar to G′, E_{c} (indicates compressive resistance) significantly increased with increasing collagen concentration (FIG. 26, Panel E). This suggests that the increased fibril density restricted interstitial fluid flow (increased hydraulic resistance).

To evaluate whether the addition of the ECFCs altered matrix mechanical properties, tests were repeated with matrices containing (2×10⁶ ECFCs/ml, gray lines; FIG. 26, Panels C and D). Results showed that cell addition significantly affected G′ but not E_{c}. With cells, G′ significantly increased at each collagen concentration except 3.5 mg/ml (p=0.2), suggesting that the effect diminished with increasing concentration and mechanical integrity. δ was also significantly affected, especially for 0.5 mg/ml matrices. These results may be explained by the cells acting as additional fibril-fibril interaction points (e.g. crosslinks formed by adherence of cells to multiple fibrils) in the cell-collagen composite material. ECFC addition did not disrupt the ability of varied collagen concentration to vary matrix biophysical properties, thus demonstrating embedded ECFCs were still exposed to similar changes in matrix stiffness and fibril density.

Increasing collagen concentration significantly increased matrix stiffness in a co-dependent fashion with increased fibril density. Collagen concentrations therefore represent significantly different biophysical microenvironments (Table 3).

TABLE 3
Summary of collagen matrix physical properties.

| Collagen | | | | |
|---|---|---|---|---|
| Concen- | | | | |
| tration | Fibril Volume | | | |
| (mg/ml) | Fraction (%) | G′ (Pa) | δ (degrees) | E_{C} (kPa) |
| 0.5 | 8.65 ± 0.29 | 3.50 ± 0.17 | 9.24 ± 0.95 | 17.17 ± 1.71 |
| 1.5 | 11.25 ± 0.47 | 16.64 ± 0.57 | 8.00 ± 0.17 | 62.51 ± 16.80 |
| 2.5 | 13.83 ± 0.60 | 28.54 ± 1.57 | 7.33 ± 0.22 | 106.63 ± 14.28 |
| 3.5 | 16.42 ± 0.61 | 46.67 ± 1.06 | 6.96 ± 0.03 | 134.25 ± 33.35 |

As herein described, differences in the local physical microenvironment had measurable impact on in vivo ECFC vessel formation. Increasing matrix collagen concentration simultaneously, as they are related, increased (linearly) fibril density and shear and compressive stiffnesses (FIG. 26, Panels A and B).

### EXAMPLE 20

### Mechanical Analysis of 3D Collagen Matrices

Viscoelastic properties were determined for each collagen matrix using a AR-2000 rheometer (TA Instruments, New Castle, Del.) with a stainless steel 40 mm diameter parallel plate geometry, humidity trap, and peltier heater in the base plate. Samples were polymerized on the rheometer for 30 min at 37° C. and matrix shear modulus was measured in oscillatory shear at 1% strain, 1 Hz (predetermined from linear viscoelastic regions). Shear moduli were decomposed into phase components: shear storage (G′) and loss (G″) moduli, related by phase shift (δ, tan δ=G″/G′). Matrix compressive behavior was measured by compressing (unconfined) matrices with the plate geometry at a rate of 10 μm/s. Stress-strain plots were generated, where compressive strain was calculated as 1-L/L₀ (engineering strain, L=height and L₀=initial height) and stress was calculated as normal force divided by plate area. Compressive moduli (E_{c}) were calculated using linear regression as the slope of the stress-strain curve from 0 to 5% strain. Mechanical analysis was performed on 3-5 independent matrices per matrix formulation with and without 2×10⁶ cells/ml.

### EXAMPLE 21

### Confocal Imaging of in Vitro Matrices

After 18 h of in vitro culture, cellularized collagen matrices were fixed with 4% paraformaldehyde. Matrices were then stained overnight with Sytox green (Invitrogen, Carlsbad, Calif.),to label nuclei, 10 μg/ml TRITC conjugated UEA-lectin (Sigma) , to label cell membranes, and Alexa-Fluor 647-phalloidin (Invitrogen), to label F-actin. Alternatively, matrices were stained overnight with 4′,6-diamidino-2-phenylindome (DAPI, Invitrogen, Carlsbad, Calif.) to label nuclei, and fluorescein isothiocyanate (FITC) conjugated to Ulex europeus type I agglutinin (UEA-1) lectin (Sigma Aldrich) to label cell membranes. Matrices were then washed with PBS, bisected, and cleared through a glycerol gradient. 3D images were collected using the confocal microscope.

ECFC Vessel Formation in Vitro:

In order to examine how the different biophysical microenvironments influenced ECFC behavior in vitro, ECFC seeded (2e⁶ cells/ml) matrices were cultured in vitro for 18 h and analyzed using confocal microscopy. Results showed the morphology of ECFCs and formation of vessel-like structures was dependent on the microenvironment. In lower concentration matrices, ECFCs appeared to form more extensive cord-like networks (FIG. 27, Panels A and B). In higher concentration matrices, ECFCs formed more independent structures with larger lumens void of matrix (FIG. 27, Panels C and D). Generally, the ECFC response at the time of implantation was similar for all matrix microenvironments (FIG. 41). The ECFC cell densities measured within the low and high concentration matrices after 18 hours of culture were not statistically different.

### EXAMPLE 22

### Transplantation of ECFCS

All animal protocols were approved by the Indiana University School of Medicine Institutional Animal Care and Use Committee. After 18 h in vitro culture, cellularized collagen matrices were bisected and implanted into the flank of 6-12 week old NOD/SCID mice (Yoder et el., 2007, Blood, 109(5): 1801-9). At 14 days, mice were euthanized and the grafts were harvested, fixed in formalin free zinc fixative (BD Biosciences), paraffin embedded, bisected, and sectioned (6 μm thick) for analysis by immunohistochemistry. A minimum of 6 matrices were implanted for each treatment group.

### EXAMPLE 23

### Immunohistochemistry of Matrices

Sections were stained as previously described. Briefly, paraffin-embedded tissue sections were deparaffinized and immersed in retrieval solution (Dako, Carpenteria, Calif.) for 20 min at 95-99° C. Slides were incubated at room temperature with anti-human CD31 (hCD31, clone JC70A, Dako) for 30 minutes followed by 10 min incubation with LASB2 link-biotin and streptavidin-HRP (Dako), then developed with DAB solution for 5 min (Dako). Slides were counter-stained with hematoxylin.

### EXAMPLE 24

### Analysis of ECFC Vessel Formation

The number of anti-human CD31 (hCD31⁺) and non-anti-human CD31 (hCD31⁻) stained blood vessels, defined as red blood cell (RBC) filled endothelial lined spaces, were counted for 16 sections per matrix explant (sections were taken from middle of explants). Additionally, hCD31⁺ non-functional tubes, defined as endothelial lined spaces which did not contain RBCs, were counted in the same manner. A minimum of 40 hCD31⁺ blood vessels were imaged for each matrix using a Leica DM 4000B microscope (Leica Microsystems, Bannockburn, Ill.) with attached Spot-SE digital camera (Diagnostic Instruments, Sterling Heights, Mich.). Section and vessel areas were measured using Metamorph (Molecular Devices, Sunnyvale, Calif.). Total vascular area was calculated as (number of hCD31⁺vessels/area)*(average hCD31⁺vessel area) for each matrix explant.

ECFC Vessel Formation in Vivo:

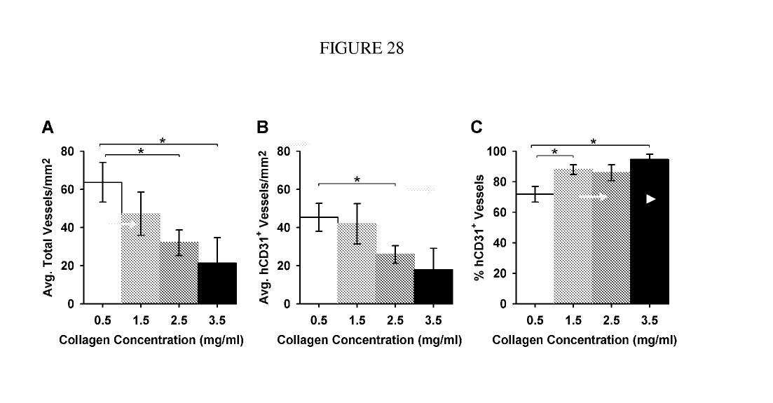

ECFCs seeded matrices were implanted into the flank of NOD/SCID mice for 14 days. Matrices were harvested and investigated for remodeling and blood vessel formation. Implanted matrices remodeled to a different extent dependent on collagen concentration (FIG. 28). Lower concentration matrices contracted to a greater degree than higher concentration matrices. Explanted matrices were surrounded by a fibrous layer (capsule) of circumferentially oriented soft tissue (host connective and muscle tissues). This capsule was used to visually define the outer edge of the matrix for quantitative analysis. In some cases, ECFC (hCD31+) and chimeric (hCD31+ and hCD31−) vessels were observed outside of the matrix. While these vessels were not included in analysis, they indicate ECFC migration and/or proliferation occurs during the active anastomosis and vessel formation processes.

While all matrices were able to direct ECFCs to form functional hCD31⁺ blood vessels which contained RBCs, they did so to a different extent (FIG. 28, bottom row). Control matrices showed no vessels, indicating ECFCs were required for vessel formation. Average total vessels per area and hCD31⁺ vessels per area decreased significantly with increasing matrix concentration from 63.7±10.4 and 45.3±7.3 vessels/mm² at 0.5 mg/ml to 21.3±13.4 and 17.9±11.3 vessels/mm² at 3.5 mg/ml (FIG. 28). The percentage of hCD31⁺ vessels increased with increasing matrix concentration from a mean of 71% to 84% of the total vessels measured.

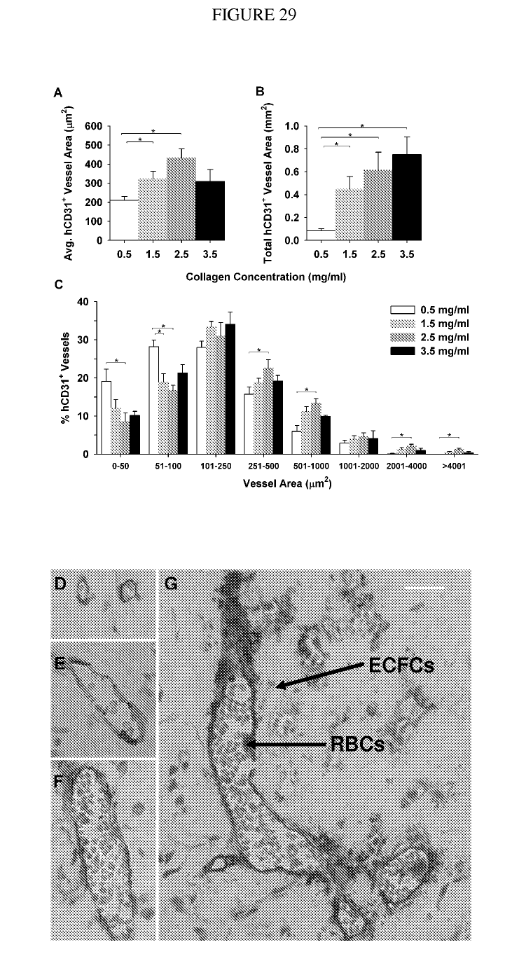

To determine the effect of matrix concentration on vessel morphology, vessel areas were measured from histology images of the explants (FIG. 29). 0.5 mg/ml matrices exhibited hCD31⁺ vessels with smaller average areas compared to 1.5 and 2.5 mg/ml matrices (FIG. 29, Panel A). This was reflected in a shifted population distribution of hCD31⁺ vessel areas (FIG. 29, Panel C). 0.5 mg/ml matrices had significantly more smaller area vessels and fewer larger area vessels compared to 2.5 mg/ml matrices (p<0.05, as indicated).

To account for the differences in matrix remodeling and vessel morphology, total hCD31⁺ vascular area was calculated and was found to significantly increase with increasing concentration. This indicates that vessel density is not a sufficient measure of vessel formation and may not accurately reflect functional vascularization (e.g. blood perfusion).

In vivo results indicating that varied matrix physical properties result in different ECFC (hCD31+) blood vessel densities and areas have been shown. Specifically, increased matrix collagen concentration resulted in larger area vessels with lower vessel density, whereas decreased concentration resulted in higher densities of smaller area vessels.

Observation of explant sizes indicated that collagen concentration varied the size of the remodeled implant, which affected vascularization. As described herein, decreased matrix collagen concentration resulted in a higher percentage of host (hCD31−) vessels, suggesting differences in host cell infiltration. Thus, varying collagen concentration and associated matrix physical properties has synergistic effects.

### EXAMPLE 25

### Collagen Separation into Monomer- and Oligomer-Rich Fractions

Type I collagen, comprising monomers and oligomers, was acid solubilized and purified from the dermis of market weight pigs as previously described (Kreger et al., 2010, Biopolymers, In Press). Subsequent fractionation of PSC into monomer- and oligomer-rich formulations was performed using two established methods: 1) polymerization in 0.03M sodium phosphate buffer, pH 7.0 containing 1M sodium chloride and 0.6M glycerol (Na et al., J. Biochemistry, 1986, 25:958-966) and 2) selective salt precipitation at 3% sodium chloride (Chandrakasan et al., J. Biol. Chem., 1976, 251: 6062-67). To demonstrate reproducibility of the glycerol-based protocol, collagen was obtained from a single pig hide (single source) or a pooled source representing 3 different pig hides (pooled source). Monomer- and oligomer- rich fractions were recombined at different ratios to create collagen formulations that varied in monomer/oligomer content and thus average polymer molecular weight (AMW).

Sodium dodecyl sulfate polyacrylamide gel electrophoresis (SDS-PAGE) was used to assess the purity and molecular composition of the collagen formulations. 12% Novex Tris-Glycine gels (Invitrogen, Carlsbad, Calif.) were used for identification of non-collagenous proteins and small molecular weight contaminants. SDS-PAGE (4%) in interrupted and uninterrupted formats and western blot analysis using mouse monoclonal antibodies specific for type I (AB6308, Abcam, Cambridge, Mass.) and type III (MAB 1343, Chemicon, Temecula) collagen were used for analysis of collagen type content (e.g. types I, III, and V). Gels were stained with Coomassie Blue or silver nitrate and imaged using a digital camera and light box. An alcian blue assay was used to assess sulfated glycosaminoglycan (GAG) content. Heparin derived from porcine intestinal mucosa (Sigma-Aldrich) was used to prepare a standard curve (1-20 heparin units/ml).

PSC Comprises Monomers and Native Oligomers

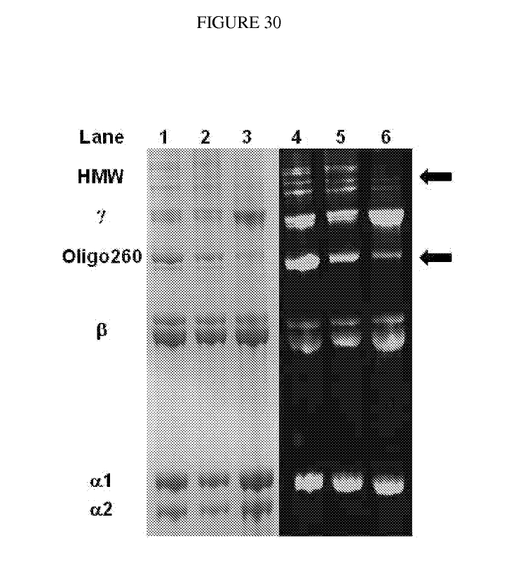

The starting material, acid-solubilized PSC, represents highly purified type I collagen with no significant contaminating glycosaminoglycans, non-collagenous proteins or other collagenous proteins (type III or type V). PSC contained a prominent band between the β and γ components with molecular weights in the range of 250-280 kDa (Oligo260), which stained positively for the collagen type I epitope, as well as high molecular weight (HMW) components (FIG. 30). Subsequent polymerization of PSC in the presence of glycerol yielded separate fractions designated monomer- and oligomer-rich, which differed in their protein banding pattern (FIG. 30) and AMW. Compared to its monomer counterpart, the oligomer fraction showed increased Oligo260 and HMW collagen bands. Intrinsic viscosity analyses indicated that the oligomer-rich and monomer-rich fractions had AMW of 603±92 kDa and 282 kDa respectively, confirming the prominence of intermolecular cross-links within the oligomer-rich fraction. On average, PSC yielded four times more oligomer-rich collagen compared to monomer-rich collagen, based upon dry weight. Fractionation results were highly reproducible whether derived from a single or batched PSC sources.

### EXAMPLE 26

### Salt Fractionation as an Alternative Method for Separating PSC Monomers and Oligomers

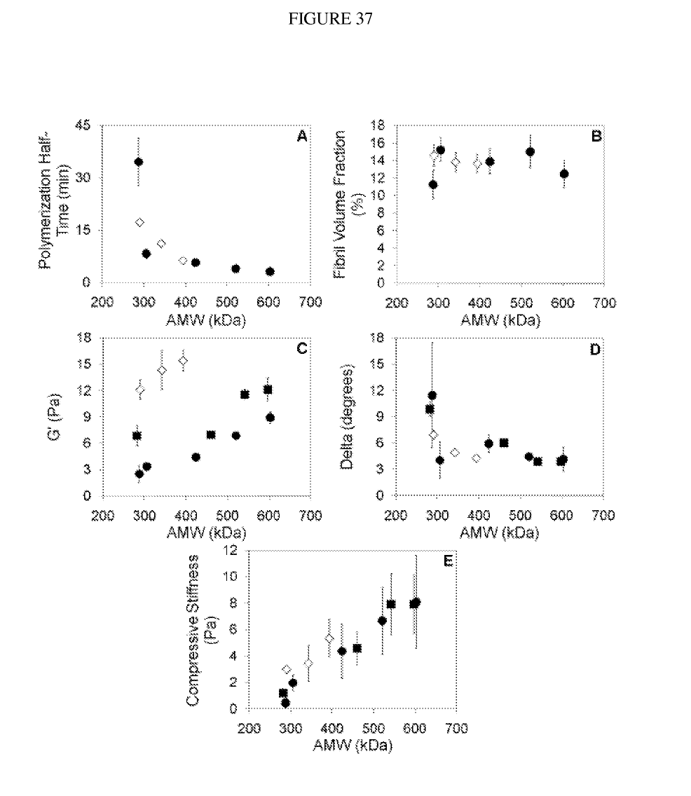

PSC monomers and oligomers also could be separated based upon differential salt precipitation. However, the fractionation efficiency of this method was less than that obtained with the glycerol-based procedure as evidenced by monomer yield and AMW for the resulting fractions. Monomer yield from salt precipitation was 10% or 2.5 times less than that achieved with glycerol. Furthermore, salt precipitation produced monomer- and oligomer-rich fractions with AMW of 290±37kDa and 394±48kDa, respectively. This range was substantially less that that obtained with the glycerol-based method where the monomer fraction was 282 kDa and the oligomer fraction was 603 kDa. However, when salt separated fractions were used to generate collagen formulations that varied in AMW similar trend in polymerization kinetics (FIG. 37A), fibril microstructure (FIG. 37B), and matrix mechanical properties (FIG. 37C-E) were observed. The slight discrepancies noted between the viscoelastic properties obtained with salt and glycerol methods can likely be attributed to molecular differences (e.g., extent of denaturation) induced during secondary processing.

### EXAMPLE 27

### Preparation of Three-Dimensional (3D) Collagen Matrices

Collagen formulations were polymerized under identical reaction conditions to produce 3D matrices. Lyophilized collagens were dissolved and diluted in 0.01 N HCl and neutralized with 10×phosphate buffered saline (PBS, 1×PBS had 0.17 M total ionic strength and pH 7.4) and 0.1 N sodium hydroxide to achieve neutral pH (7.4) and final collagen concentrations ranging from 0.5 to 1 mg/ml. Neutralized collagen solutions were kept on ice prior to the induction of polymerization by warming to 37° C. Due to the increased viscosity of collagen solutions, positive displacement pipettes (Microman, Gilson, Inc., Middleton, Wis.) were used to accurately pipette all collagen solutions.

### EXAMPLE 28

### Quantification of Intrinsic Viscosity and AMW

The AMW of the collagen formulations was determined by intrinsic viscosity measurements. In brief, apparent viscosities of collagen solutions in 0.01 N HCl were measured on an AR2000 rheometer (TA Instruments, New Castle, DE) with a cone geometry (40 mm, 2° cone angle). Viscosities for solutions representing collagen concentrations of 0.1 to 0.3 mg/mL were measured at shear rates of 100-1500 s⁻¹ at 10° C. The weight-average AMW was calculated using the Mark-Houwink equation, |η|=kM^{a}, where a=1.8 and k was determined for each shear rate assuming a monomer-rich AMW of 282 kDa. AMW was extrapolated to zero shear rate. Three replicates were performed at the three concentrations.

### EXAMPLE 29

### Analysis of Collagen Polymerization Kinetics

A turbidimetric assay was used to analyze the polymerization kinetics of each collagen formulation. Neutralized collagen solutions at 0.5, 0.7, and 1 mg/mL concentrations were transferred to a pre-warmed (37° C.) 384-well plate. Absorbance at 405 nm was measured using a SpectraMax M5 (Molecular Devices, Sunnyvale, Calif.) every 30 seconds for two hours at 37° C. Kinetic parameters calculated from the sigmoidal-shaped turbidity curves included lag time (x-intercept of line formed from one-quarter and one-half final absorbance values), polymerization rate during growth phase (slope of the line formed from previous points), and polymerization half-time (time at which absorbance equals half the final absorbance value). At least four replicates were performed at each concentration tested.

Oligomer-rich Collagen Shows Rapid Polymerization Independent of Collagen Concentration

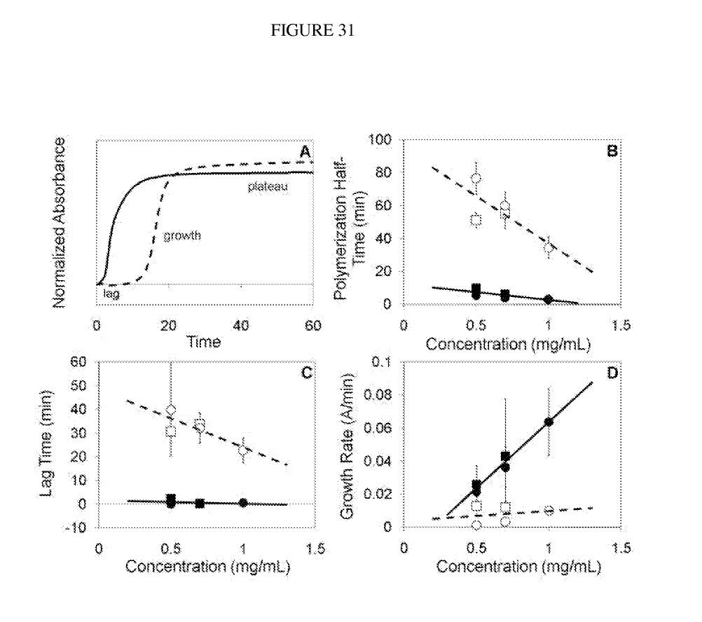

Turbidimetric analyses were used to compare polymerization kinetics of monomer- and oligomer-rich collagens as a function of collagen concentration. All formulations yielded expected sigmoidal-shaped relationships between A405 and time with definable “lag”, “growth”, and “plateau” phases (FIG. 31, Panel A). Measured kinetic parameters for oligomer-rich and monomer-rich formulations showed distinct dependence on collagen concentration (p<0.05). The monomer-rich fraction showed a nearly 50% reduction in polymerization half-time and a 30% reduction in lag time as collagen concentration was increased from 0.5 to 1.0 mg/ml (FIGS. 31, Panels B and C). In contrast, half-time and lag time values for the oligomer-rich fraction were significantly shorter (p<0.05) and remained relatively constant over the concentration range tested, with values of 3 minutes and 1 minute, respectively. Unlike the monomer fraction, the oligomer-containing formulations exhibited a linear increase in growth phase rate as a function of concentration (FIG. 31, Panel D). However, this change had only minor effects on the overall polymerization half-time, which, in general, appeared to more closely parallel lag phase duration.

Polymerization Rate Decreases and Polymerization Half-Time Increases with Collagen AMW

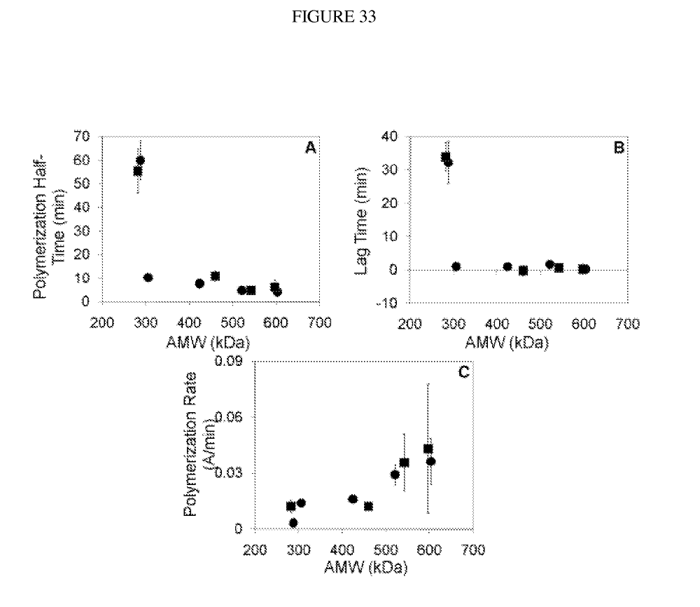

Turbidimetric analyses also were used to determine how collagen polymerization kinetics varied with monomer/oligomer content or AMW. AMW was systematically adjusted by mixing monomer- and oligomer- rich fractions in different proportions. As AMW was increased from 282 kDa to 306 kDa a dramatic step change in mean polymerization half-time from 55±9 minutes to 10±1 minutes was observed (FIG. 33, Panel A). Polymerization half-times remained consistently short for solutions with AMW greater than 306 kDa. A similar trend was observed for lag time as a function of AMW (FIG. 33, Panel B) again demonstrating lag phase is a primary determinant of the overall polymerization half-time. The growth phase rate was found to increase linearly with AMW (FIG. 33, Panel C).

### EXAMPLE 30

### Analysis of Collagen Fibril Microstructure

Confocal reflection microscopy (CRM) was used to collect high-resolution, 3D images of the matrices in their native, hydrated state. Matrices were polymerized in Lab-Tek chambered coverglass slides (Nunc, Thermo Fisher Scientific, Rochester, N.Y.) at concentrations of 0.5, 0.7, and 1.0 mg/mL for 2 hours at 37° C. CRM was performed on an Olympus Fluoview FV1000 confocal system adapted to an Olympus IX81 inverted microscope with a 60×UPlanSApo water immersion objective (Olympus, Tokyo, Japan). Image stacks were collected at 5 random locations within each of 5 independent matrices per formulation (n=25). Three microstructure parameters, fibril density, fibril diameter, and pore size were quantified from the images. Fibril density and diameter were measured as described previously (Kreger et al., 2009, Matrix Biol., 28: 336-346). Diameter measurements were made on randomly chosen single, non-dividing fibrils (10 fibrils per each of 5 independent matrices; n=50). Pore size was calculated from 2D projections of 50-slice image stacks representing a total thickness of 5 μm using Image-Pro Plus 5.1 software (Media Cybernetics Inc., Bethesda, Md.). The images were binarized by thresholding and pore size was quantified as the cross-sectional areas encompassed by collagen fibrils.

Imaging via transmission electron microscopy (TEM) was performed on aliquots of the collagen polymerization reaction taken at various periods of time. Samples were placed on copper support grids with carbon-coated formvar films and allowed to settle for 30 seconds. The samples were stained using 1% phosphotungustic acid (pH 7.2, KOH), air-dried, and imaged using a Philips CM-100 TEM (FEI Company, Hillsboro, Oreg.) with 80 kV accelerating voltage. Images were captured on Kodak SO-163 Electron Image film and scanned into digital format at 600 dpi.

Collagen AMW Affects Interfibril Branching and Matrix Pore Size But Not Fibril Density or Diameter

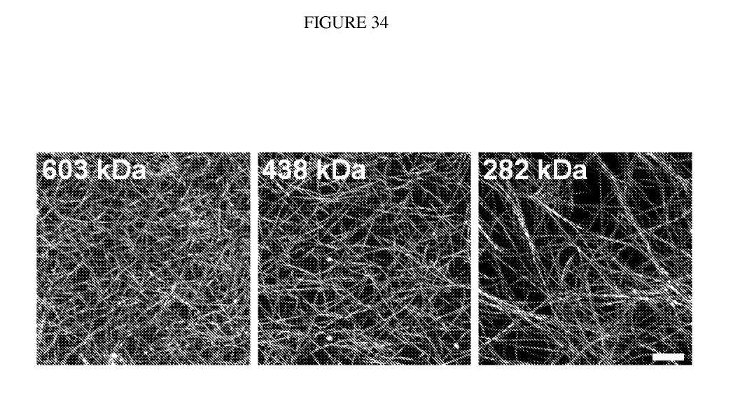

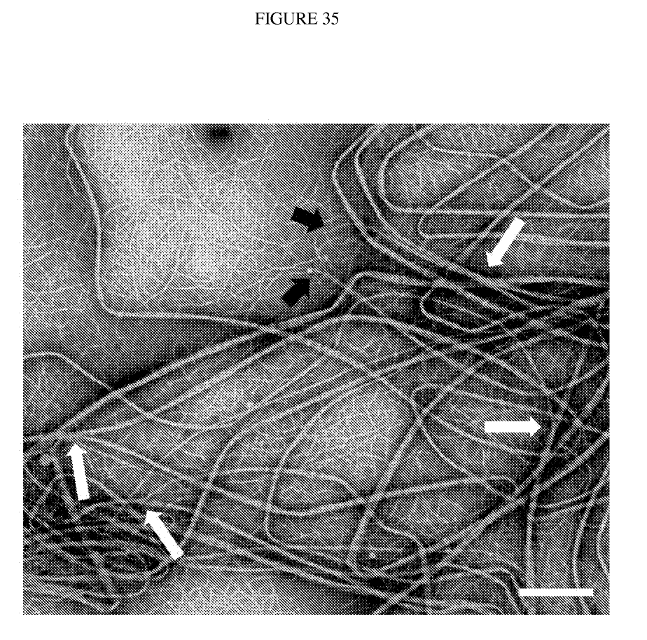

The variation of AMW and therefore monomer/oligomer content was found to influence not only polymerization kinetics but also the overall fibril architecture of polymerized matrices. CRM was used to visualize the 3D fibril microstructure of unprocessed, hydrated specimens (FIG. 34) and provided the basis for quantification of fibril diameter, fibril density, and projected pore size (Table 4). Quantification of fibril density and diameter showed no dependence on AMW. In fact, fibril density and diameter remained relatively constant with ranges of 8-14% and 300-450 nm, respectively. The most profound difference in fibril microstructure as determined qualitatively and quantitatively from CRM images was that the mean projected pore size decreased and showed less variance with increasing AMW (Table 4). Time-based analysis of fibril assembly using high resolution TEM indicated that banded fibrils formed more rapidly and with a larger number of interfibril branches within oligomer-rich fractions compared to their monomeric counterparts (FIG. 35). Banded fibrils appeared to form and elongate via microfibril condensation at their tapered ends. In contrast, interfibril branch formation appeared to result from lateral associations between developing fibrils, events which also have been documented during in-vivo tendon development. Thus, systematic increases in AMW were found to decrease the projected pore size and increase the extent of interfibril branching with little to no effect on fibril density and fibril diameter.

TABLE 4
Summary of microstructural properties of 3D matrices
prepared with collagen formulations with varied AMW or
monomer/oligomer content. All matrices were polymerized
under similar conditions at 0.7 mg/ml collagen concentration.

| Collagen | Fibril | Fibril | |
|---|---|---|---|
| AMW | Diameter | Density | Pore Size |
| (kDa) | (nm) | (%) | (μm²) |
| 288 | 370 ± 50 | 8.1 ± 2.6 | 15.1 ± 13.8 |
| 306 | 352 ± 31 | 11.5 ± 2.3 | 2.7 ± 1.4 |
| 424 | 349 ± 31 | 13.6 ± 1.7 | 1.9 ± 0.8 |
| 521 | 377 ± 29 | 14.6 ± 1.6 | 1.7 ± 0.7 |
| 603 | 341 ± 32 | 13.1 ± 2.3 | 1.8 ± 0.8 |

Matrix Stiffness is Positively Correlated with Collagen AMW

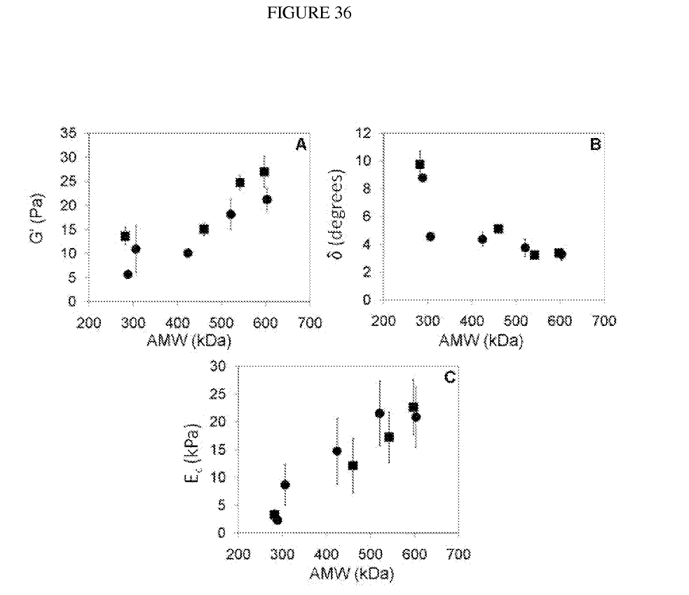

The functional significance of increased interfibril branching and decreased pore size as occurs with increasing collagen AMW was documented by measurement of matrix viscoelastic properties. Despite collagen concentration, and therefore fibril density and diameter, remaining constant, G′ increased and δ decreased with increasing AMW (FIGS. 36, Panels A and B). This enhanced ability to store elastic energy was further corroborated by an impressive increase in E, (FIG. 36, Panel C). Values ranged from 3.3±0.9 kPa for 282 kDa matrices to 20.8±5.4 kPa for 603 kDa matrices, indicating that hydraulic conductivity or permeability of matrices was reciprocally related to monomer/oligomer content. In general, the highly consistent mechanical properties established previously for PSC lots prepared from different pig hides translated into good reproducibility of mechanical testing results obtained as a function of AMW for both single and pooled source batches.

### EXAMPLE 31

### Analysis of Matrix Mechanical Properties in Shear and Unconfined Compression

Viscoelastic properties of polymerized collagen matrices (0.5, 0.7, and 1 mg/ml final collagen concentration) were measured in both oscillatory shear and unconfined compression on a stress-controlled AR2000 rheometer (TA Instruments, New Castle, Del.) adapted with a stainless-steel, 40-mm diameter, parallel-plate geometry. A shear strain sweep from 0.01 to 5% strain at 1 Hz was used to measure the shear modulus (reported values at 1% strain). The controlling software calculated shear storage (G′, elastic/solid component representing stored, recoverable energy) and loss (G″, viscous/fluid component representing energy permanently lost during deformation) moduli, which are related by phase shift (δ) as tan (G″/G′). Following the strain sweep, normal force was measured in response to compressive strain generated by depressing the geometry at a rate of 20 μm/s (strain rate 2.76%/s). The compressive modulus (E_{c}) was calculated using linear regression of the stress-strain curve slope from 10 to 30% strain. Shear and compression tests were performed on at least 4 independent matrices per formulation (n≧4).

Oligomer-rich Collagen Matrices Show Increased Mechanical Integrity Compared to Monomer-rich Collagen Matrices

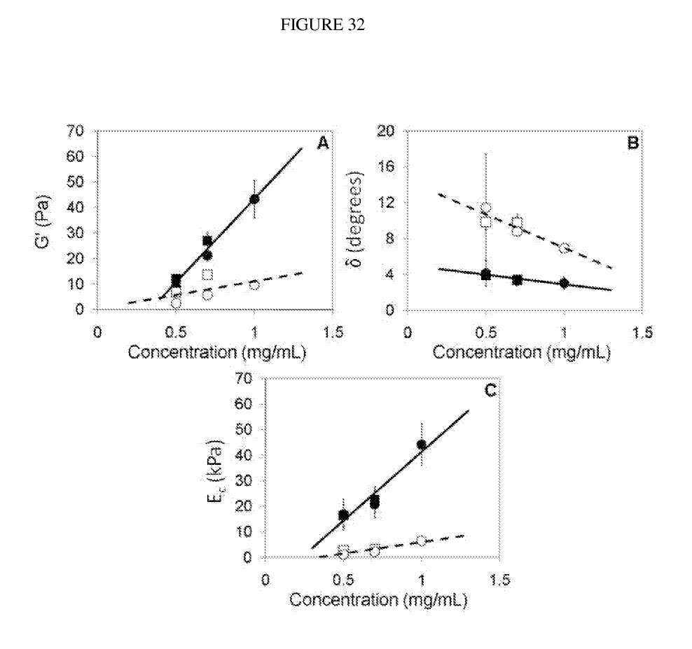

Further insight into the polymerization potential of monomer- and oligomer-rich collagens as a function of collagen concentration was gained through mechanical testing of polymerized matrices. Since collagen matrices represent composite viscoelastic materials, the mechanical testing was determined using oscillatory shear and unconfined compression loading formats. Although matrix stiffness measures G′ and E_{c} were positively correlated with concentration for both matrix types, those formed with the oligomer-rich fraction showed a significantly greater increase in G′ and E, over the concentration range tested (p<0.05; FIG. 32, Panel A and C). On the other hand, phase shift δ, an indicator of matrix fluidity, for monomer-rich matrices decreased linearly with concentration while values for oligomer-rich matrices remained relatively low and constant (FIG. 32, Panel B).

### EXAMPLE 32

### Preparation of 3D Cellularized Tissue Constructs

Human umbilical cord blood ECFCs were obtained from the laboratory of Mervin Yoder (Indiana University School of Medicine) and cultured according to established methods (Ingram et al., 2004, Blood, 104(9): 2752-60). Cellularized tissue constructs were prepared as previously described (Pizzo et al., 2005, J Appl Physiol, 98(5): 1909-21.; Kreger et al., 2010, Biopolymers, In Press) with minor modifications. ECFCs (5×10⁵ cells/ml) were suspended in neutralized solutions of monomer-rich or oligomer-rich collagen formulations that were matched on final collagen concentration (1 mg/ml) or matrix stiffness as measured by G′. Collagen-cell suspensions were pipetted into 24-well plates (0.5 ml/well), allowed to polymerize at 37° C., and then cultured in complete endothelial cell growth medium (EGM-2, Lonza, Walkersville, Md.) at 37° C. and 5% CO₂.

Systematic Variation of Collagen AMW Modulates ECFC Vacuole and Lumen Formation In Vitro

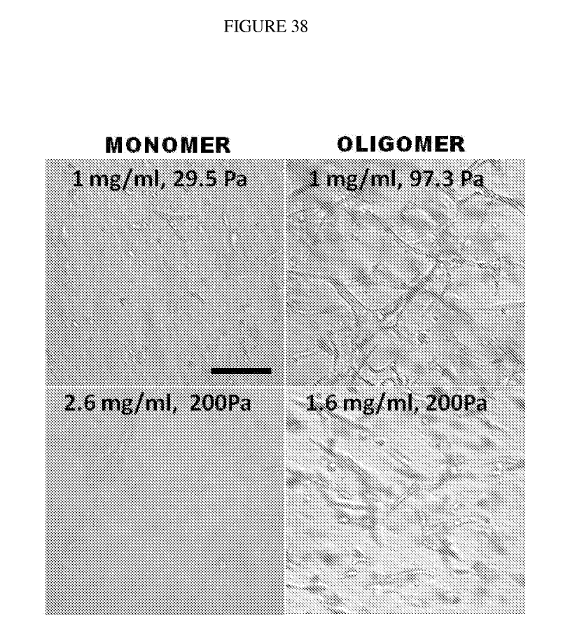

In-vitro and in-vivo vessel morphogenesis by differentiated endothelial cells or EPC populations can be modulated by varying the collagen concentration of the surrounding matrix. In the present study, ECFCs could sense and respond to physical properties differences of matrices produced by varying AMW of collagen formulations independent of concentration. ECFCs were seeded at the same cell density within pig skin derived monomer and oligomer collagen matrices that were matched in either collagen concentration (1 mg/ml) or matrix G′ (200 Pa). Interestingly, in all cases the extent of vacuole and lumen formation was greatest for oligomer matrices (FIG. 38) indicative of differences in cell-matrix interactions. It was also noted, that for both monomer and oligomer formulations, the extent of vacuolization and vessel network formation decreased with increasing collagen concentration and therefore matrix G′ over the ranges tested (FIG. 38). These results were consistent with findings made when ECFC were cultured and compared in PSC and commercial monomeric collagen preparations (Becton Dickinson rat tail collagen; data not shown).

### EXAMPLE 33

### Comparison of Monomer-Rich and Oligomer-Rich Matrices Seeded with ECFCS

Preparation of Pig Skin Monomer-Rich Collagen

A pig skin collagen formulation enriched in monomers (single collagen molecules) was prepared by acid-solubilization of pig skin collagen in 0.5M acetic acid. The resultant collagen was further purified by secondary salt precipitation to yield a collagen monomer formulation.

Preparation of Three-Dimensional (3D) Collagen Matrices

Collagen formulations were polymerized under identical reaction conditions to produce 3D matrices. Lyophilized collagens were dissolved and diluted in 0.01 N HCl and neutralized with 10× phosphate buffered saline (PBS, 1× PBS had 0.17 M total ionic strength and pH 7.4) and 0.1 N sodium hydroxide to achieve neutral pH (7.4) and final collagen concentrations ranging from 0.5 to 1 mg/ml. Neutralized collagen solutions were kept on ice prior to the induction of polymerization by warming to 37° C. Due to the increased viscosity of collagen solutions, positive displacement pipettes (Microman, Gilson, Inc., Middleton, Wis.) were used to accurately pipette collagen solutions.

Preparation of 3D Cellularized Tissue Constructs

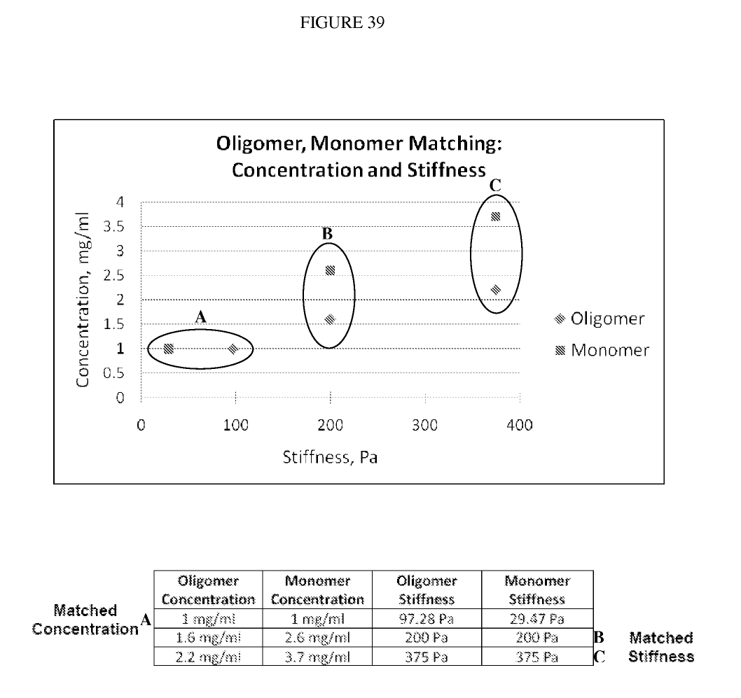

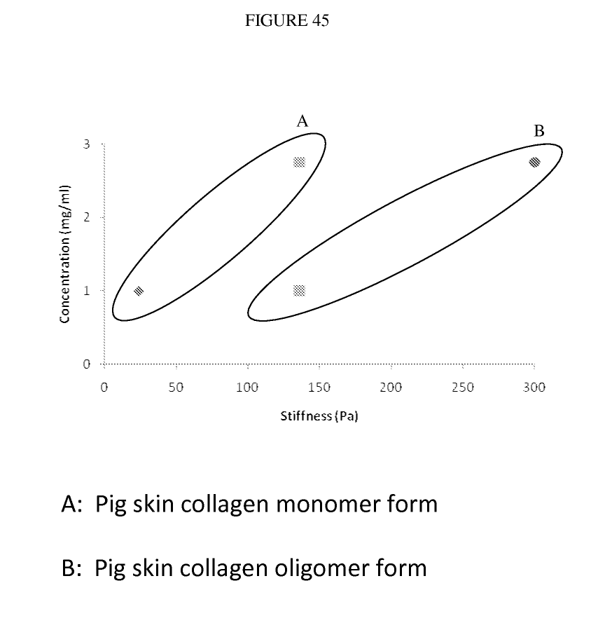

Human umbilical cord blood endothelial colony forming cells were obtained from the laboratory of Mervin Yoder (Indiana University School of Medicine) and cultured according to established methods (Ingram et al., 2004, Blood, 104(9): 2752-60). Cellularized tissue constructs were prepared as previously described (Pizzo et al., 2005, J Appl Physiol. 98(5): 1909-21.; Kreger et al., 2010, Biopolymers, In Press; each incorporated herein by reference) with minor modifications. ECFCs (5×10⁵ cells/ml) were suspended in neutralized solutions of monomer-rich or oligomer-rich PSC formulations that were matched on final collagen concentration (1 mg/ml) or matrix stiffness as measured by G′. Matrices were matched for collagen concentration or stiffness as shown (FIGS. 39 and 45). Collagen-cell suspensions were pipetted into 24-well plates (500 μl/well) or 96-well plates (58 μl/well), allowed to polymerize at 37° C., and then cultured in complete endothelial cell growth medium (EGM-2, Lonza, Walkersville, Md.) at 37° C. and 5% CO₂.

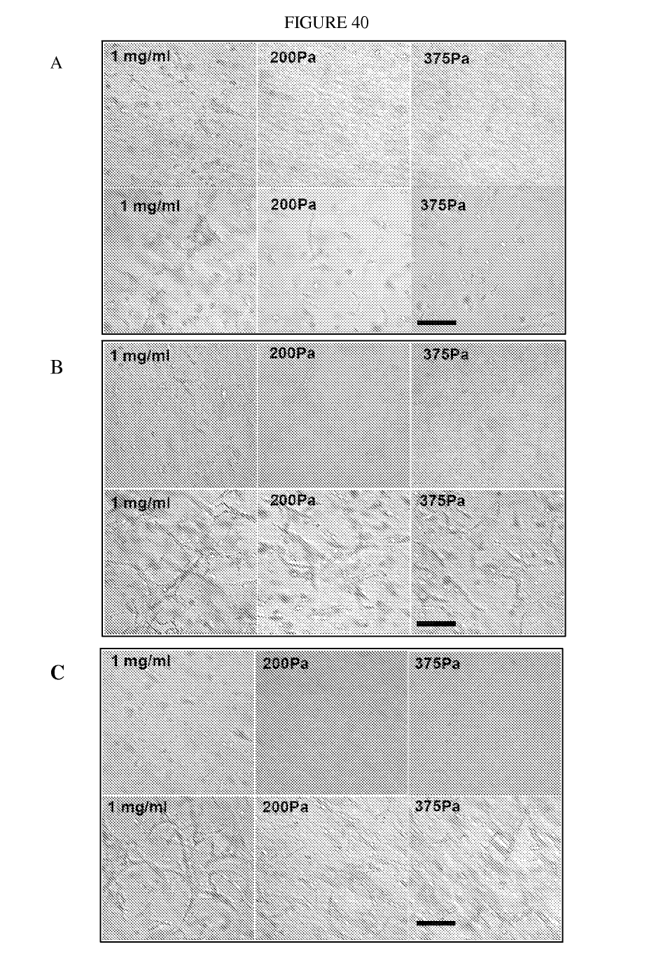

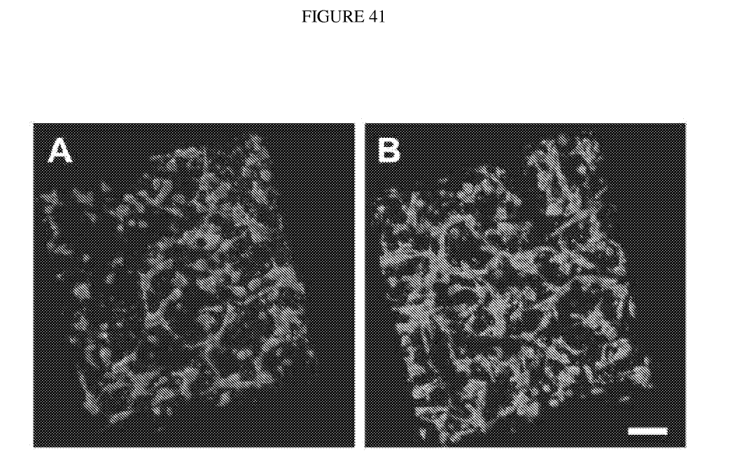

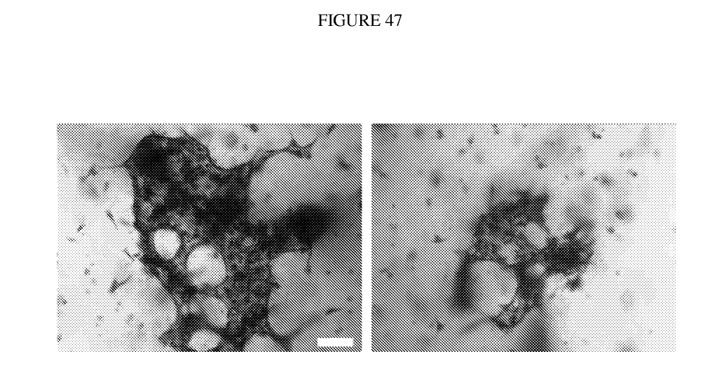

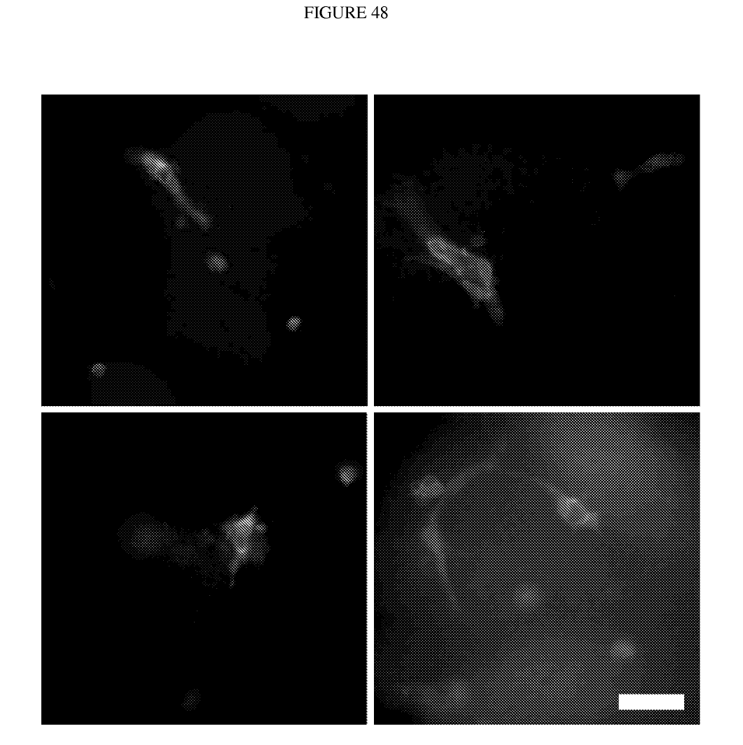

Cell growth was monitored using light microscopy at days 1, 2, and 3 (FIG. 40, Panels A, B, and C, respectively). Alternatively, matrices were stained overnight with 4′,6-diamidino-2-phenylindome (DAPI, Invitrogen, Carlsbad, Calif.) to label nuclei, and fluorescein isothiocyanate (FITC) conjugated to Ulex europeus type I agglutinin (UEA-1) lectin (Sigma Aldrich) to label cell membranes. Matrices were then washed with PBS, bisected, and cleared through a glycerol gradient. 3D images were collected using the confocal microscope (FIG. 48). Vacuoles merge to yield larger lumens containing cells within the lumens (FIG. 47).

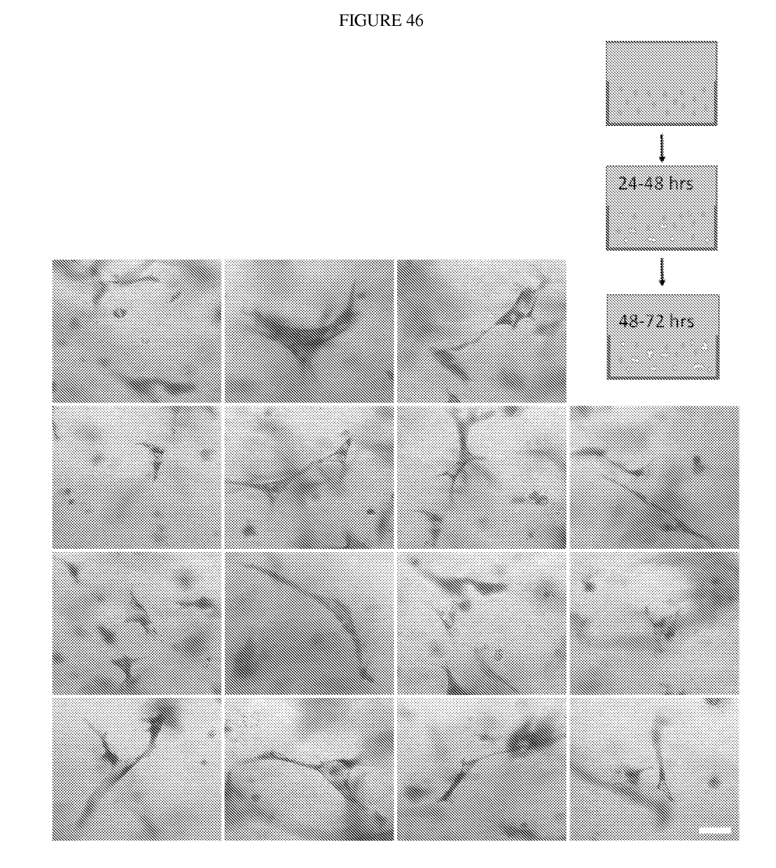

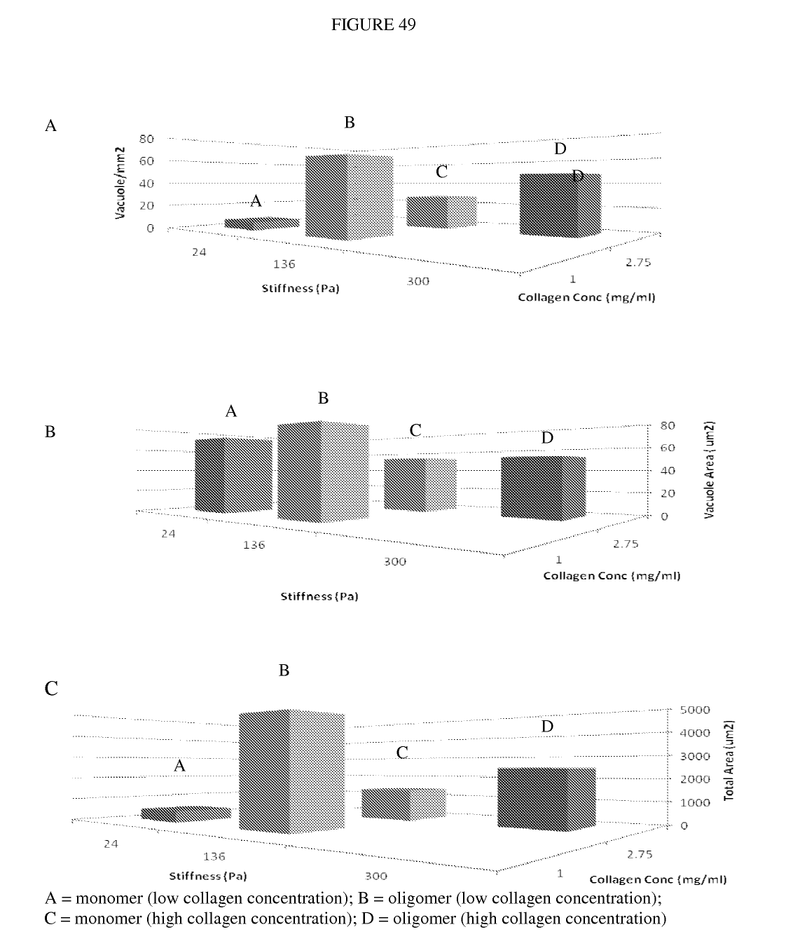

For visualization and quantification of vacuole number and size, in-vitro tissue constructs were stained with Toluidine Blue O. Quantification was performed on bright field images (100×) representing the central 9-16 fields within each of three different z-planes per construct (FIG. 46). Metamorph was used to trace vacuoles to yield both vacuole number and vacuole area. Vacuole density (FIG. 49, Panel A), average vacuole area (FIG. 49, Panel B), and total vacuole area (FIG. 49, Panel C) were determined.

### EXAMPLE 34

### ECFC Vessel Formation in Vivo Using Rat Tail or Pig Skin Collagen Matrices

After 18 h in vitro culture, cellularized collagen matrices were bisected and implanted into the flank of 6-12 week old NOD/SCID mice (Yoder et el., 2007, Blood, 109(5): 1801-9). Collagen matrices were prepared using rat tail collagen or pig skin collagen preparations as described. Matrices were matched for collagen concentration and stiffness, as shown in table 5. At 14 days, mice were euthanized and the grafts were harvested, fixed in formalin free zinc fixative (BD Biosciences), paraffin embedded, bisected, and sectioned (6 μm thick) for analysis by immunohistochemistry. A minimum of 6 matrices were implanted for each treatment group.

TABLE 5

| Collagen | Concentration | Shear Storage |
|---|---|---|
| Source | (mg/ml) | Modulus (Pa) |
| RTC | 0.5 | 18 |
| PSC | 0.5 | 20 |
| RTC | 1.5 | 33 |
| RTC | 2.5 | 35 |
| RTC | 3.5 | 49 |
| PSC | 1.5 | 150 |
| PSC | 2.5 | 475 |

Sections were stained as previously described. Briefly, paraffin-embedded tissue sections were deparaffinized and immersed in retrieval solution (Dako, Carpenteria, Calif.) for 20 min at 95-99° C. Slides were incubated at room temperature with anti-human CD31 (hCD31, clone JC70A, Dako) for 30 minutes followed by 10 min incubation with LASB2 link-biotin and streptavidin-HRP (Dako), then developed with DAB solution for 5 min (Dako). Slides were counter-stained with hematoxylin.

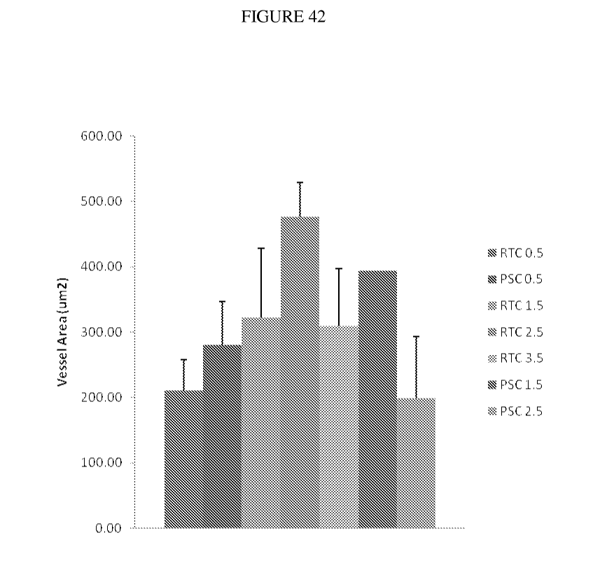

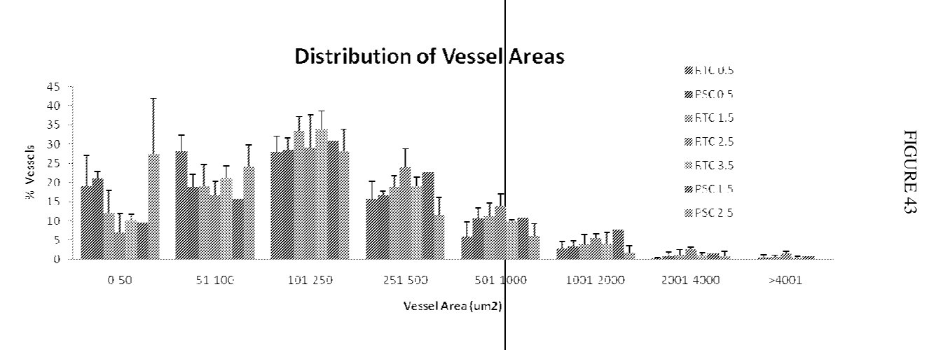

The number of anti-human CD31 (hCD31⁺) and non-anti-human CD31 (hCD31⁻) stained blood vessels, defined as red blood cell (RBC) filled endothelial lined spaces, were counted for 16 sections per matrix explant (sections were taken from middle of explants). Additionally, hCD31⁺ non-functional tubes, defined as endothelial lined spaces which did not contain RBCs, were counted in the same manner. A minimum of 40 hCD31⁺ blood vessels were imaged for each matrix using a Leica DM 4000B microscope (Leica Microsystems, Bannockburn, Ill.) with attached Spot-SE digital camera (Diagnostic Instruments, Sterling Heights, Mich.). Section and vessel areas were measured using Metamorph (Molecular Devices, Sunnyvale, Calif.). Total vascular area was calculated as (number of hCD31⁺ vessels/area)*(average hCD31⁺ vessel area) for each matrix explant (FIG. 42). A quantitative analysis of the vessel area arranged as a distribution related to collagen concentration of rat tail (RTC) versus pig skin collagen (PSC) was performed (FIG. 43).

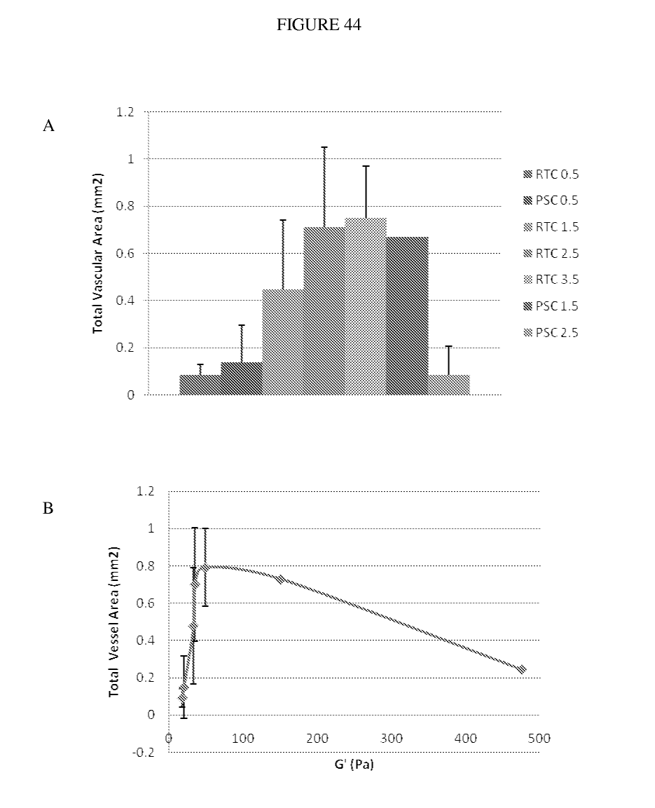

To determine the effect of matrix concentration on vessel morphology, vessel areas were measured from histology images of the explants (FIG. 44, Panels A and B).

### EXAMPLE 35

### Statistical Analysis

All values are presented as mean±standard error (SE). Statistical significance between groups was determined by Student's t test (pooled, two-tail comparisons) using SAS software (SAS Institute Inc, Cary, N.C.). A value of p<0.05 was considered significant. To determine differences among treatment groups the general linear model (GLM) procedure was used to conduct unbalanced analysis of variance (ANOVA, in some cases a Kruske-Wallis ANOVA for nonparametric distributions) and perform multiple comparisons of least squares means using the Tukey-Kramer method. In some cases, pairwise comparisons were made using Student t-tests. Differences were considered statistically significant when p<0.05.

1. A method of regulating vessel density within an engineered purified collagen-based matrix composition, said method comprising the steps of engineering a purified collagen-based matrix comprising collagen fibrils; and seeding the matrix with endothelial progenitor cells wherein said seeding results in vessel formation and an increase in of vessel density within the matrix with decreasing collagen concentration.

2-81. (canceled)
